(19) United States
(12) Patent Application Publication
(10) Pub. No.: US 2026/0125486 A1
(43) Pub. Date: May 7, 2026

(54) METHODS AND COMPOSITIONS FOR CANCER TREATMENT

(71) Applicants: GENENTECH, INC., South San Francisco, CA (US); PFIZER INC., New York, NY (US)

(72) Inventors: Erica Ehrha PARK, South San Francisco, CA (US); Monica Etelina SUSILO, South San Francisco, CA (US); James Niall COOPER, South San Francisco, CA (US); Mohamed ELMELIEGY, San Diego, CA (US); Gregory Joseph FINN, Cambridge, MA (US)

(73) Assignees: GENENTECH, INC., South San Francisco, CA (US); PFIZER INC., New York, NY (US)

(21) Appl. No.: 19/426,894

(22) Filed: Dec. 19, 2025

Related U.S. Application Data
(63) Continuation of application No. PCT/US2024/034915, filed on Jun. 21, 2024.
(60) Provisional application No. 63/509,612, filed on Jun. 22, 2023.
(60) Provisional application No. 63/600,286, filed on Nov. 17, 2023.

(51) Int. Cl. C07K 16/28; A61K 31/138; A61K 31/167; A61K 31/573; A61K 39/00; A61P 35/00; C07K 16/24

(52) U.S. Cl. CPC C07K 16/2878; A61K 31/138; A61K 31/167; A61K 31/573; A61P 35/00; C07K 16/248; C07K 16/2809; C07K 16/283; A61K 2039/545; C07K 2317/24; C07K 2317/31; C07K 2317/524; C07K 2317/526; C07K 2317/565; C07K 2317/71

(57) ABSTRACT

The present application provides methods of treatment of cancers, such as multiple myelomas, with anti-fragment crystallizable receptor-like 5 (FcRH5)/anti-cluster of differentiation 3 (CD3) bispecific antibodies and anti-B cell maturation factor (BCMA)/anti-CD3 bispecific antibodies.

This application is a continuation of International Application No. PCT/US2024/034915, filed on Jun. 21, 2024, which claims priority to U.S. Provisional Patent Application No. 63/509,612, filed on Jun. 22, 2023, and to U.S. Provisional Patent Application No. 63/600,286, filed on Nov. 17, 2023, the entire contents of which are incorporated herein by reference.

### SEQUENCE LISTING

The instant application contains a Sequence Listing which has been submitted electronically in XML format and is hereby incorporated by reference in its entirety. Said XML copy, created on Dec. 15, 2025, is named “00B206_1694.xml” and is 65,974 bytes in size.

### FIELD OF THE INVENTION

The present application relates to the treatment of cancers, such as B cell proliferative disorders. More specifically, the application concerns the treatment of human subjects having multiple myeloma (MM) using an anti-fragment crystallizable receptor-like 5 (FcRH5)/anti-cluster of differentiation 3 (CD3) bispecific antibody and an anti-B-cell maturation antigen (BCMA)/anti-CD3 bispecific antibody.

### BACKGROUND

Cancer remains one of the deadliest threats to human health. In the U.S., cancer affects more than 1.7 million new patients each year and is the second leading cause of death after heart disease, accounting for approximately one in four deaths.

Hematologic cancers, in particular, are the second leading cause of cancer-related deaths. Hematologic cancers include multiple myeloma (MM), an incurable neoplasm characterized by the proliferation and accumulation of malignant plasma cells in the bone marrow that leads to the overproduction of monoclonal proteins (M-proteins) detectable in the blood or urine of most patients. A diagnosis of MM affects approximately 30,000 people every year in the United States, and approximately 160,000 people are diagnosed with MM annually worldwide. End-organ damage resulting from MM includes hypercalcemia, renal insufficiency, anemia, and lytic bone lesions. MM remains incurable despite advances in treatment, with an estimated median survival of 8-10 years for standard-risk and 2-3 years for high-risk myeloma, even with aggressive treatments such as autologous stem cell transplantation (SCT). Despite the significant improvement in patient survival over the past 20 years, only 10-15% of patients achieve or exceed expected survival compared with the matched general population.

Therefore, there is a need for improved treatment regimens for MM and other cancers (e.g., hematologic cancers).

### SUMMARY OF THE INVENTION

Provided herein are, inter alia, methods of treating a subject having a cancer (e.g., MM), compositions for use, and related articles of manufacture.

In one aspect, provided herein is a method of treating a subject having a multiple myeloma (MM), the method including administering to the subject (i) an effective amount of a first bispecific antibody that specifically binds to fragment crystallizable receptor-like 5 (FcRH5) and cluster of differentiation 3 (CD3) and (ii) an effective amount of a second bispecific antibody that specifically binds to B-cell maturation antigen (BCMA) and CD3.

In another aspect, provided herein is a bispecific antibody that specifically binds to FcRH5 and CD3 for use in treatment of a subject having an MM, the treatment comprising administration of a first bispecific antibody that specifically binds to FcRH5 and CD3 and a second bispecific antibody that specifically binds to BCMA and CD3 to the subject.

In another aspect, provided herein is a bispecific antibody that specifically binds to BCMA and CD3 for use in treatment of a subject having an MM, the treatment comprising administration of a first bispecific antibody that specifically binds to FcRH5 and CD3 and a second bispecific antibody that specifically binds to BCMA and CD3 to the subject.

In some aspects, the subject has relapsed or refractory (R/R) MM.

In some aspects, the subject has diagnosis of R/R MM according to International Myeloma Working Group (IMWG) criteria.

In some aspects, the first bispecific antibody and the second bispecific antibody are administered to the subject in a dosing regimen including: (i) a first phase including one or more dosing cycles; (ii) a second phase including one or more dosing cycles; and, optionally, (iii) a third phase including one or more dosing cycles.

In some aspects, the dosing regimen includes the first phase and the second phase but does not include the third phase.

In some aspects, the dosing regimen includes the first phase, the second phase, and the third phase.

In some aspects, each dosing cycle of the first phase, the second phase, and/or the third phase is a 14-day dosing cycle.

In some aspects, the second phase is a 28-day dosing cycle. In some aspects, the second bispecific antibody is administered to the subject on Day 1 of each dosing cycle of the second phase at a target dose of 76 mg.

In some aspects, the first phase includes dosing cycles C1 to C26, and the second phase comprises dosing cycles C27 and beyond.

In some aspects each dosing cycle after at about one year from a first dosing of the second bispecific antibody administered to the subject is 28-days.

In some aspects, each dosing cycle of the second bispecific antibody administered to the subject on the 27^{th} dosing cycle and thereafter is a 28-days.

In some aspects, each dosing cycle of the first phase and the second phase is a 14-day dosing cycle, and each dosing cycle of the third phase is a 28-day dosing cycle.

In some aspects, the second bispecific antibody is administered to the subject on Day 1 and Day 8 of each dosing cycle of the first phase; is administered to the subject on Day 1 of each dosing cycle of the second phase; and/or is administered to the subject on Day 1 of each dosing cycle of the third phase. In some aspects, the third phase starts about one year from the first target dose of the second bispecific antibody administered to the subject. In some aspects, the 27^{th} dosing cycle (C27) is the first dosing cycle of the third phase. In some aspects, the method further includes a pre-phase, prior to the first phase, including one or more dosing cycles.

In some aspects, the pre-phase includes one dosing cycle (C1).

In some aspects, the C1 of the pre-phase is: (i) approximately a 15-day dosing cycle; or (ii) approximately a 22-day dosing cycle.

In some aspects, the C1 of the pre-phase is approximately a 15-day dosing cycle.

In some aspects, the pre-phase includes administering the first bispecific antibody to the subject on Day 9, Day 10, or Day 11 of the C1.

In some aspects, the first bispecific antibody is administered to the subject on Day 9, Day 10, or Day 11 of the C1 at a step-up dose of 3.6 mg. In some aspects, the pre-phase includes administering the second bispecific antibody to the subject on (i) Day 1; (ii) Day 3, Day 4, or Day 5; and (iii) Day 8.

In some aspects, the pre-phase comprises administering the second bispecific antibody to the subject on (i) Day 1; (ii) Day 4; and (iii) Day 8. In some aspects, the second bispecific antibody is administered to the subject on Day 1 at a first step-up dose of 12 mg.

In some aspects, the second bispecific antibody is administered to the subject on Day 3, Day 4, or Day 5 at a second step-up dose of 32 mg.

In some aspects, the second bispecific antibody is administered to the subject on Day 8 at a target dose of 76 mg.

In some aspects, the C1 of the pre-phase is approximately a 22-day dosing cycle.

In some aspects, the pre-phase includes administering the first bispecific antibody to the subject on Day 16, Day 17, or Day 18 of the C1.

In some aspects, the first bispecific antibody is administered to the subject on Day 16, Day 17, or Day 18 of the C1 at a step-up dose of 3.6 mg.

In some aspects, the pre-phase includes administering the second bispecific antibody to the subject on (i) Day 1; (ii) Day 3, Day 4, or Day 5; (iii) Day 8; and (iv) Day 15.

In some aspects, the second bispecific antibody is administered to the subject on Day 1 at a first step-up dose of 12 mg.

In some aspects, the second bispecific antibody is administered to the subject on Day 3, Day 4, or Day 5 at a second step-up dose of 32 mg.

In some aspects, the second bispecific antibody is administered to the subject on Day 8 at a target dose of 76 mg.

In some aspects, the second bispecific antibody is administered to the subject on Day 15 at a target dose of 76 mg.

In some aspects, the first phase includes at least one dosing cycle, at least two dosing cycles, at least three dosing cycles, at least four dosing cycles, or at least five dosing cycles.

In some aspects, the first phase includes a first 14-day dosing cycle (C1).

In some aspects, a target dose of the first bispecific antibody is administered to the subject during the first phase.

In some aspects, the first phase includes administering the target dose of the first bispecific antibody to the subject on Day 2, Day 3, or Day 4 of the C1.

In some aspects, the target dose of the first bispecific antibody is 60 mg.

In some aspects, the target dose of the first bispecific antibody is 60 mg administered for C1 through C8 of the first phase Q2W and thereafter the target dose of the first bispecific antibody is 60 mg administered Q4W.

In some aspects, the target dose of the first bispecific antibody is 105 mg.

In some aspects, the target dose of the first bispecific antibody is 105 mg administered for C1 through C8 of the first phase Q2W and thereafter the target dose of the first bispecific antibody is 60 mg administered Q4W.

In some aspects, the target dose of the first bispecific antibody is 132 mg.

In some aspects, a target dose of the second bispecific antibody is administered to the subject during the first phase.

In some aspects, the first phase includes administering the target dose of the second bispecific antibody to the subject on Day 1 of the C1.

In some aspects, the first phase includes administering the target dose of the second bispecific antibody to the subject on Day 1 and Day 8 of the C1.

In some aspects, the target dose of the second bispecific antibody is 76 mg.

In some aspects, the target dose of the second bispecific antibody is 76 mg administered for C1 through C8 of the first phase Q2W and thereafter the target dose of the second bispecific antibody is 76 mg administered Q4W.

In some aspects, the second phase includes at least one dosing cycle, at least two dosing cycles, at least three dosing cycles, at least four dosing cycles, at least five dosing cycles, at least six dosing cycles, at least seven dosing cycles, at least eight dosing cycles, at least nine dosing cycles, at least ten dosing cycles, at least eleven dosing cycles, at least twelve dosing cycles, at least thirteen dosing cycles, at least fourteen dosing cycles, at least fifteen dosing cycles, at least sixteen dosing cycles, at least seventeen dosing cycles, at least eighteen dosing cycles, at least nineteen dosing cycles, at least twenty dosing cycles, at least twenty-one dosing cycles, at least twenty-two dosing cycles, at least twenty-three dosing cycles, at least twenty-four dosing cycles, or at least twenty-five dosing cycles.

In some aspects, the second phase includes a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25).

In some aspects, each dosing cycle of the second phase is a 14-day dosing cycle.

In some aspects, a target dose of the first bispecific antibody is administered to the subject during the second phase.

In some aspects, the second phase includes administering the target dose of the first bispecific antibody to the subject on Day 1 of each dosing cycle.

In some aspects, the second phase includes administering the target dose of the first bispecific antibody to the subject on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and/or the C25 of the second phase.

In some aspects, the target dose of the first bispecific antibody is 60 mg.

In some aspects, the target dose of the first bispecific antibody is 132 mg.

In some aspects, the target dose if the first bispecific antibody is 105 mg.

In some aspects, the target dose of the first bispecific antibody is 60 mg administered for C1 through C8 of the second phase Q2W and thereafter the target dose of the first bispecific antibody is 60 mg administered Q4W.

In some aspects, the target dose of the first bispecific antibody is 105 mg administered for C1 through C8 of the second phase Q2W and thereafter the target dose of the first bispecific antibody is 105 mg administered Q4W.

In some aspects, a target dose of the second bispecific antibody is administered to the subject during the second phase.

In some aspects, the second phase includes administering the target dose of the second bispecific antibody to the subject on Day 1 of each dosing cycle.

In some aspects, the second phase includes administering the target dose of the second bispecific antibody to the subject on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and/or the C25 of the second phase.

In some aspects, the second phase includes administering the target dose of the second bispecific antibody to the subject on Day 1 and Day 8 of each dosing cycle.

In some aspects, the second phase includes administering the target dose of the second bispecific antibody to the subject on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and/or the C25 of the second phase.

In some aspects, the subject achieves an IWMG response category of partial response (PR) or better with a response persisting for at least two months, the dosing frequency of the second bispecific antibody is changed to every two weeks (Q2W).

In some aspects, if after 6 months the subject achieves an IWMG response category of complete response (CR), the dosing frequency of the first bispecific antibody and the second bispecific antibody is changed from every two weeks (Q2W) to every four weeks (Q4W).

In some aspects, if the subject achieves an IWMG response category of partial response (PR) with a response persisting for at least two months, the dosing frequency of the second bispecific antibody is changed from every two weeks (Q2W) to every four weeks (Q4W).

In some aspects, if the subject subsequently has an increase of disease burden not yet qualifying as progressive disease according to IWMG criteria, the dosing frequency is changed to every week (QW).

In some aspects, the second phase includes administering the target dose of the second bispecific antibody to the subject on Day 8 of each dosing cycle.

In some aspects, the second phase includes administering the target dose of the second bispecific antibody to the subject on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and/or the C25.

In some aspects, the target dose of the second bispecific antibody is 76 mg.

In some aspects, the target dose of the second bispecific antibody is 76 mg administered for C1 through C8 of the second phase Q2W and thereafter the target dose of the second bispecific antibody is 76 mg administered Q4W.

In some aspects, the method includes administering the first bispecific antibody to the subject until disease progression, unacceptable toxicity, or up to 26 total dosing cycles.

In some aspects, the third phase includes one or more dosing cycles.

In some aspects, the third phase includes between 1 and 130 dosing cycles.

In some aspects, each dosing cycle of the third phase is a 14-day dosing cycle.

In some aspects, a target dose of the second bispecific antibody is administered to the subject during the third phase.

In some aspects, the third phase includes administering the target dose of the second bispecific antibody to the subject on Day 1 of each dosing cycle of the third phase.

In some aspects, the third phase includes administering the target dose of the second bispecific antibody to the subject on Day 1 and Day 8 of each dosing cycle of the third phase.

In some aspects, the subject achieves an IWMG response category of partial response (PR) or better with a response persisting for at least two months, and the dosing frequency of the second bispecific antibody is changed to every two weeks (Q2W).

In some aspects, if the subject subsequently has an increase of disease burden not yet qualifying as progressive disease according to IWMG criteria, the dosing frequency is changed to every week (QW). In some aspects, if after 6 months the subject achieves an IWMG response category of complete response (CR), the dosing frequency of the first bispecific antibody and the second bispecific antibody is changed from every two weeks (Q2W) to every four weeks (Q4W).

In some aspects, if the subject achieves an IWMG response category of partial response (PR) with a response persisting for at least two months, the dosing frequency of the second bispecific antibody is changed from every two weeks (Q2W) to every four weeks (Q4W).

In some aspects, the target dose of the second bispecific antibody is 76 mg.

In some aspects, the second bispecific antibody is administered to the subject until disease progression or unacceptable toxicity.

In some aspects, the first bispecific antibody is administered to the subject intravenously.

In some aspects, the second bispecific antibody is administered to the subject subcutaneously.

In some aspects, the method further includes administering a corticosteroid to the subject.

In some aspects, the method further includes administering a corticosteroid to the subject during the first phase, the second phase, and/or the third phase.

In some aspects, the corticosteroid is administered to the subject during the first phase 1 hour (±15 minutes) before administration of the first bispecific antibody or the second bispecific antibody.

In some aspects: (i) the subject experienced cytokine release syndrome (CRS) with a prior administration of the first bispecific antibody or the second bispecific antibody, and the corticosteroid is administered to the subject during the second phase 1 hour (±15 minutes) before administration of the first bispecific antibody or the second bispecific antibody; and/or (ii) the subject experienced CRS with a prior administration of the first bispecific antibody or the second bispecific antibody, and the corticosteroid is administered to the subject during the third phase 1 hour (±15 minutes) before administration of the second bispecific antibody.

In some aspects, the method further includes administering a corticosteroid to the subject during the pre-phase.

In some aspects, the corticosteroid is administered to the subject during the C1 of the pre-phase 1 hour (±15 minutes) before administration of the first bispecific antibody or the second bispecific antibody.

In some aspects, the corticosteroid is dexamethasone or methylprednisolone.

In some aspects, the corticosteroid is dexamethasone.

In some aspects, the dexamethasone is administered to the subject at a dose of about 20 mg.

In some aspects, the methylprednisolone is administered to the subject at a dose of about 80 mg.

In some aspects, the corticosteroid is administered to the subject intravenously.

In some aspects, the first bispecific antibody that specifically binds to FcRH5 and CD3 includes an anti-FcRH5 arm including a first binding domain including the following six hypervariable regions (HVRs): (a) an HVR-H1 including the amino acid sequence of RFGVH (SEQ ID NO: 1); (b) an HVR-H2 including the amino acid sequence of VIWRGGSTDYNAAFVS (SEQ ID NO: 2); (c) an HVR-H3 including the amino acid sequence of HYYGSSDYALDN (SEQ ID NO: 3); (d) an HVR-L1 including the amino acid sequence of KASQDVRNLVV (SEQ ID NO: 4); (e) an HVR-L2 including the amino acid sequence of SGSYRYS (SEQ ID NO: 5); and (f) an HVR-L3 including the amino acid sequence of QQHYSPPYT (SEQ ID NO: 6).

In some aspects, the first bispecific antibody that specifically binds to FcRH5 and CD3 includes an anti-FcRH5 arm including a first binding domain including (a) a heavy chain variable (VH) domain including an amino acid sequence having at least 95% sequence identity to the amino acid sequence of SEQ ID NO: 7; (b) a light chain variable (VL) domain including an amino acid sequence having at least 95% sequence identity to the amino acid sequence of SEQ ID NO: 8; or (c) a VH domain as in (a) and a VL domain as in (b).

In some aspects, the first binding domain includes a VH domain including an amino acid sequence of SEQ ID NO: 7 and a VL domain including an amino acid sequence of SEQ ID NO: 8.

In some aspects, the first bispecific antibody that specifically binds to FcRH5 and CD3 includes an anti-CD3 arm including a second binding domain including the following six HVRs: (a) an HVR-H1 including the amino acid sequence of SYYIH (SEQ ID NO: 9); (b) an HVR-H2 including the amino acid sequence of WIYPENDNTKYNEKFKD (SEQ ID NO: 10); (c) an HVR-H3 including the amino acid sequence of DGYSRYYFDY (SEQ ID NO: 11); (d) an HVR-L1 including the amino acid sequence of KSSQSLLNSRTRKNYLA (SEQ ID NO: 12); (e) an HVR-L2 including the amino acid sequence of WTSTRKS (SEQ ID NO: 13); and (f) an HVR-L3 including the amino acid sequence of KQSFILRT (SEQ ID NO: 14).

In some aspects, the first bispecific antibody that specifically binds to FcRH5 and CD3 includes an anti-CD3 arm including a second binding domain including (a) a VH domain including an amino acid sequence having at least 95% sequence identity to the amino acid sequence of SEQ ID NO: 15; (b) a VL domain including an amino acid sequence having at least 95% sequence identity to the amino acid sequence of SEQ ID NO: 16; or (c) a VH domain as in (a) and a VL domain as in (b).

In some aspects, the second binding domain includes a VH domain including an amino acid sequence of SEQ ID NO: 15 and a VL domain including an amino acid sequence of SEQ ID NO: 16.

In some aspects, the first bispecific antibody that specifically binds to FcRH5 and CD3 includes an anti-FcRH5 arm including a heavy chain polypeptide (H1) and a light chain polypeptide (L1) and an anti-CD3 arm including a heavy chain polypeptide (H2) and a light chain polypeptide (L2), and wherein: (a) H1 includes the amino acid sequence of SEQ ID NO: 35; (b) L1 includes the amino acid sequence of SEQ ID NO: 36; (c) H2 includes the amino acid sequence of SEQ ID NO: 37; and (d) L2 includes the amino acid sequence of SEQ ID NO: 38.

In some aspects, the first bispecific antibody that specifically binds to FcRH5 and CD3 includes an aglycosylation site mutation.

In some aspects, the aglycosylation site mutation reduces effector function of the bispecific antibody.

In some aspects, the aglycosylation site mutation is a substitution mutation.

In some aspects, the first bispecific antibody that specifically binds to FcRH5 and CD3 includes a substitution mutation in the Fc region that reduces effector function.

In some aspects, the first bispecific antibody that specifically binds to FcRH5 and CD3 is a monoclonal antibody.

In some aspects, the first bispecific antibody that specifically binds to FcRH5 and CD3 is a chimeric antibody.

In some aspects, the first bispecific antibody that specifically binds to FcRH5 and CD3 is a humanized antibody.

In some aspects, the first bispecific antibody that specifically binds to FcRH5 and CD3 is an antibody fragment that specifically binds FcRH5 and CD3.

In some aspects, the antibody fragment that specifically binds FcRH5 and CD3 is selected from the group consisting of Fab, Fab′-SH, Fv, scFv, and (Fab′)2 fragments.

In some aspects, the first bispecific antibody that specifically binds to FcRH5 and CD3 is a full-length antibody.

In some aspects, the first bispecific antibody that specifically binds to FcRH5 and CD3 is an IgG antibody.

In some aspects, the IgG antibody that specifically binds to FcRH5 and CD3 is an IgG1 antibody. In some aspects, the first bispecific antibody that specifically binds to FcRH5 and CD3 includes one or more heavy chain constant domains, wherein the one or more heavy chain constant domains are selected from a first CH1 (CH11) domain, a first CH2 (CH21) domain, a first CH3 (CH31) domain, a second CH1 (CH12) domain, second CH2 (CH22) domain, and a second CH3 (CH32) domain.

In some aspects, at least one of the one or more heavy chain constant domains is paired with another heavy chain constant domain.

In some aspects, the CH31 and CH32 domains each include a protuberance or cavity, and wherein the protuberance or cavity in the CH31 domain is positionable in the cavity or protuberance, respectively, in the CH32 domain.

In some aspects, the CH31 and CH32 domains meet at an interface between the protuberance and cavity.

In some aspects, the CH21 and CH22 domains each include a protuberance or cavity, and wherein the protuberance or cavity in the CH21 domain is positionable in the cavity or protuberance, respectively, in the CH22 domain.

In some aspects, the CH21 and CH22 domains meet at an interface between said protuberance and cavity.

In some aspects, the anti-FcRH5 arm includes the protuberance and the anti-CD3 arm includes the cavity.

In some aspects, a CH3 domain of the anti-FcRH5 arm includes a protuberance including a T366W amino acid substitution mutation (EU numbering) and a CH3 domain of the anti-CD3 arm includes a cavity including T366S, L368A, and Y407V amino acid substitution mutations (EU numbering).

In some aspects, the first bispecific antibody that specifically binds to FcRH5 and CD3 is cevostamab.

In some aspects, the second bispecific antibody that specifically binds to BCMA and CD3 includes an anti-BCMA arm including a first binding domain including the following six HVRs: (a) an HVR-H1 including the amino acid sequence of GFTFSSY (SEQ ID NO: 40), SYPMS (SEQ ID NO: 39), or GFTFSSYPMS (SEQ ID NO: 41); (b) an HVR-H2 including the amino acid sequence of GGSGGS (SEQ ID NO: 43) or AIGGSGGSLPYADIVKG (SEQ ID NO: 42); (c) an HVR-H3 including the amino acid sequence of YWPMDI (SEQ ID NO: 44); (d) an HVR-L1 including the amino acid sequence of RASQSVSSSYLA (SEQ ID NO: 45); (e) an HVR-L2 including the amino acid sequence of DASIRAT (SEQ ID NO: 46); and (f) an HVR-L3 including the amino acid sequence of QQYQSWPLT (SEQ ID NO: 47).

In some aspects, the second bispecific antibody that specifically binds to BCMA and CD3 includes an anti-BCMA arm including a first binding domain including (a) a VH domain including an amino acid sequence having at least 95% sequence identity to the amino acid sequence of SEQ ID NO: 48; (b) a VL domain including an amino acid sequence having at least 95% sequence identity to the amino acid sequence of SEQ ID NO: 49; or (c) a VH domain as in (a) and a VL domain as in (b).

In some aspects, the first binding domain of the second bispecific antibody includes a VH domain including an amino acid sequence of SEQ ID NO: 48 and a VL domain including an amino acid sequence of SEQ ID NO: 49.

In some aspects, the second bispecific antibody that specifically binds to BCMA and CD3 includes an anti-CD3 arm including a second binding domain including the following six HVRs: (a) an HVR-H1 including the amino acid sequence of GFTFSDY (SEQ ID NO: 53), DYYMT (SEQ ID NO: 52), or GFTFSDYYMT (SEQ ID NO: 54); (b) an HVR-H2 including the amino acid sequence of RNRARGYT (SEQ ID NO: 56) or FIRNRARGYTSDHNPSVKG (SEQ ID NO: 55); (c) an HVR-H3 including the amino acid sequence of DRPSYYVLDY (SEQ ID NO: 57); (d) an HVR-L1 including the amino acid sequence of KSSQSLFNVRSRKNYLA (SEQ ID NO: 58); (e) an HVR-L2 including the amino acid sequence of WASTRES (SEQ ID NO: 59); and (f) an HVR-L3 including the amino acid sequence of KQSYDLFT (SEQ ID NO: 60).

In some aspects, the second bispecific antibody that specifically binds to BCMA and CD3 includes an anti-CD3 arm including a second binding domain including (a) a VH domain including an amino acid sequence having at least 95% sequence identity to the amino acid sequence of SEQ ID NO: 61; (b) a VL domain including an amino acid sequence having at least 95% sequence identity to the amino acid sequence of SEQ ID NO: 62; or (c) a VH domain as in (a) and a VL domain as in (b).

In some aspects, the second binding domain of the second bispecific antibody includes a VH domain including an amino acid sequence of SEQ ID NO: 61 and a VL domain including an amino acid sequence of SEQ ID NO: 62.

In some aspects, the bispecific antibody that specifically binds to BCMA and CD3 includes an anti-BCMA arm including a heavy chain polypeptide (H1) and a light chain polypeptide (L1) and an anti-CD3 arm including a heavy chain polypeptide (H2) and a light chain polypeptide (L2), and wherein: (a) H1 includes the amino acid sequence of SEQ ID NO: 50; (b) L1 includes the amino acid sequence of SEQ ID NO: 51; (c) H2 includes the amino acid sequence of SEQ ID NO: 63; and (d) L2 includes the amino acid sequence of SEQ ID NO: 64.

In some aspects, the second bispecific antibody includes a substitution mutation in the Fc region that reduces effector function.

In some aspects, the second bispecific antibody is a monoclonal, chimeric, or humanized antibody.

In some aspects, the second bispecific antibody that specifically binds to BCMA and CD3 is an antibody fragment that specifically binds BCMA and CD3.

In some aspects, the antibody fragment that specifically binds BCMA and CD3 is selected from the group consisting of Fab, Fab′-SH, Fv, scFv, and (Fab′)2 fragments.

In some aspects, the second bispecific antibody is a full-length antibody.

In some aspects, the second bispecific antibody is an IgG antibody.

In some aspects, the IgG antibody is an IgG2 antibody.

In some aspects, the second bispecific antibody includes first and second respective human IgG2 constant regions including amino acid modifications at positions 223 and 228, or 223, 225 and 228, respectively, in the hinge region, and at position 368 or 409, respectively, in the CH3 region (EU numbering).

In some aspects, the first human IgG2 constant region includes C223E, P228E, and L368E amino acid substitution mutations (EU numbering).

In some aspects, the second human IgG2 constant region includes C223R, E225R, P228R, and K409R amino acid substitution mutations (EU numbering).

In some aspects, the second bispecific antibody that specifically binds to BCMA and CD3 is elranatamab.

In some aspects, the first bispecific antibody and/or the second bispecific antibody are administered to the subject concurrently with one or more additional therapeutic agents.

In some aspects, the first bispecific antibody and/or the second bispecific antibody are administered to the subject prior to the administration of one or more additional therapeutic agents.

In some aspects, the first bispecific antibody and/or the second bispecific antibody is administered to the subject subsequent to the administration of one or more additional therapeutic agents.

In some aspects, the one or more additional therapeutic agents include an effective amount of an anti-IL-6 antibody.

In some aspects, the anti-IL-6 antibody is tocilizumab.

In some aspects, the subject has a CRS event, and the method further includes treating the symptoms of the CRS event while suspending treatment with the first bispecific antibody and the second bispecific antibody.

In some aspects, the method further includes administering to the subject an effective amount of tocilizumab to treat the CRS event.

In some aspects, the CRS event does not resolve or worsens within 24 hours of treating the symptoms of the CRS event, and the method further including administering to the subject one or more additional doses of tocilizumab to manage the CRS event.

In some aspects, tocilizumab is administered to the subject by intravenous infusion.

In some aspects: (a) the subject weighs ≥30 kg, and tocilizumab is administered to the subject at a dose of 8 mg/kg; or (b) the subject weighs <30 kg, and tocilizumab is administered to the subject at a dose of 12 mg/kg.

In some aspects, tocilizumab is administered to the subject 2 hours before administration of the first bispecific antibody and/or the second bispecific antibody.

In some aspects, the one or more additional therapeutic agents include an effective amount of acetaminophen or paracetamol.

In some aspects, acetaminophen or paracetamol is administered to the subject at a dose of between 500 mg to 1000 mg.

In some aspects, acetaminophen or paracetamol is administered to the subject orally.

In some aspects, the one or more additional therapeutic agents include an effective amount of diphenhydramine.

In some aspects, diphenhydramine is administered to the subject at a dose of between 25 mg to 50 mg.

In some aspects, diphenhydramine is administered to the subject orally.

In some aspects, the method includes pre-medication with the following agents prior to administration of the first bispecific antibody and/or the second bispecific antibody to the subject: (i) a corticosteroid; (ii) acetaminophen or paracetamol; and/or (iii) diphenhydramine.

In some aspects, the method includes a pre-phase, and the corticosteroid is administered to the subject 1 hour (±15 minutes) prior to any administration of the first bispecific antibody and/or the second bispecific antibody during the pre-phase.

In some aspects, the method includes a first phase, and the corticosteroid is administered to the subject 1 hour (±15 minutes) prior to any administration of the first bispecific antibody and/or the second bispecific antibody during the first phase.

In some aspects, the method includes a second phase, wherein the subject has experienced CRS with a prior administration of the first bispecific antibody and/or the second bispecific antibody, and the corticosteroid is administered to the subject 1 hour (±15 minutes) prior to any administration of the first bispecific antibody and/or the second bispecific antibody during the second phase.

In some aspects, the method includes a third phase, wherein the subject has experienced CRS with a prior administration of the first bispecific antibody and/or the second bispecific antibody, and the corticosteroid is administered to the subject 1 hour (±15 minutes) prior to any administration of the second bispecific antibody during the third phase.

In some aspects, the corticosteroid is dexamethasone or methylprednisolone.

In some aspects, the corticosteroid is dexamethasone.

In some aspects, the dexamethasone is administered to the subject at a dose of about 20 mg.

In some aspects, the methylprednisolone is administered to the subject at a dose of about 80 mg. In some aspects, the corticosteroid is administered to the subject intravenously.

In some aspects, the acetaminophen or paracetamol is administered to the subject at a dose of between 500 mg to 1000 mg.

In some aspects, the acetaminophen or paracetamol is administered to the subject orally.

In some aspects, diphenhydramine is administered to the subject at a dose of between 25 mg to 50 mg.

In some aspects, diphenhydramine is administered to the subject orally.

In another aspect, provided herein is a method of treating a subject having an R/R MM, the method including administering to the subject cevostamab and elranatamab in a dosing regimen including: (i) a pre-phase including an approximately 15-day dosing cycle (C1); (ii) a first phase, following the pre-phase, including a first dosing cycle (C1), wherein the C1 of the first phase is a 14-day dosing cycle; (iii) a second phase, following the first phase, including a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, including one or more dosing cycles, wherein cevostamab is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another aspect, provided herein is cevostamab for use in treatment of a subject having an R/R MM, the treatment comprising administering to the subject cevostamab and elranatamab in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another aspect, provided herein is elranatamab for use in treatment of a subject having an R/R MM, the treatment comprising administering to the subject cevostamab and elranatamab in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another aspect, provided herein is a method of treating a subject having an R/R MM, the method including administering to the subject cevostamab and elranatamab in a dosing regimen including: (i) a pre-phase including an approximately 22-day dosing cycle (C1); (ii) a first phase, following the pre-phase, including a first dosing cycle (C1), wherein the C1 of the first phase is a 14-day dosing cycle; (iii) a second phase, following the first phase, including a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, including one or more dosing cycles, wherein cevostamab is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another aspect, provided herein is cevostamab for use in treatment of a subject having an R/R MM, the treatment comprising administering to the subject cevostamab and elranatamab in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another aspect, provided herein is elranatamab for use in treatment of a subject having an R/R MM, the treatment comprising administering to the subject cevostamab and elranatamab in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another aspect, provided herein is a method of treating a subject having an R/R MM, the method including administering to the subject cevostamab and elranatamab in a dosing regimen including: (i) a first phase including a first dosing cycle (C1), wherein the C1 of the first phase is a 14-day dosing cycle; (ii) a second phase, following the first phase, including a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, including one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase.

In another aspect, provided herein is cevostamab for use in treatment of a subject having an R/R MM, the treatment comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase.

In another aspect, provided herein is elranatamab for use in treatment of a subject having an R/R MM, the treatment comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase.

In another aspect, provided herein is a method of treating a subject having an R/R MM, the method including administering to the subject cevostamab and elranatamab in a dosing regimen including: (i) a first phase including a first dosing cycle (C1), wherein the C1 of the first phase is a 14-day dosing cycle; (ii) a second phase, following the first phase, including a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, including one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another aspect, provided herein is cevostamab for use in treatment of a subject having an R/R MM, the treatment comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another aspect, provided herein is elranatamab for use in treatment of a subject having an R/R MM, the treatment comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In some aspects, the method further includes a pre-phase prior to the first phase.

In some aspects, the pre-phase includes an approximately 15-day dosing cycle (C1), wherein: cevostamab is administered to the subject at a step-up dose of 3.6 mg on Day 9, 10, or 11 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase.

In some aspects, the pre-phase includes an approximately 22-day dosing cycle (C1), wherein: cevostamab is administered to the subject at a step-up dose of 3.6 mg on Day 16, Day 17, or Day 18 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase.

In some aspects, the subject is a human subject.

### BRIEF DESCRIPTION OF THE DRAWINGS

FIG. 1 is a schematic diagram showing the overall design of the GO43979 study. DLT=dose-limiting toxicity; ISC=internal safety committee.

FIG. 2 is a schematic diagram showing the GO43979 initial study treatment dosing schedule (Option 1 in FIG. 1). Cevos=cevostamab.

FIG. 3 is a schematic diagram showing the GO43979 study treatment dosing schedule with pre-phase mitigation (Option 2 in FIG. 1).

FIG. 4 is a schematic diagram showing the GO43979 dosing schedule for target dose levels B and C (FIG. 1).

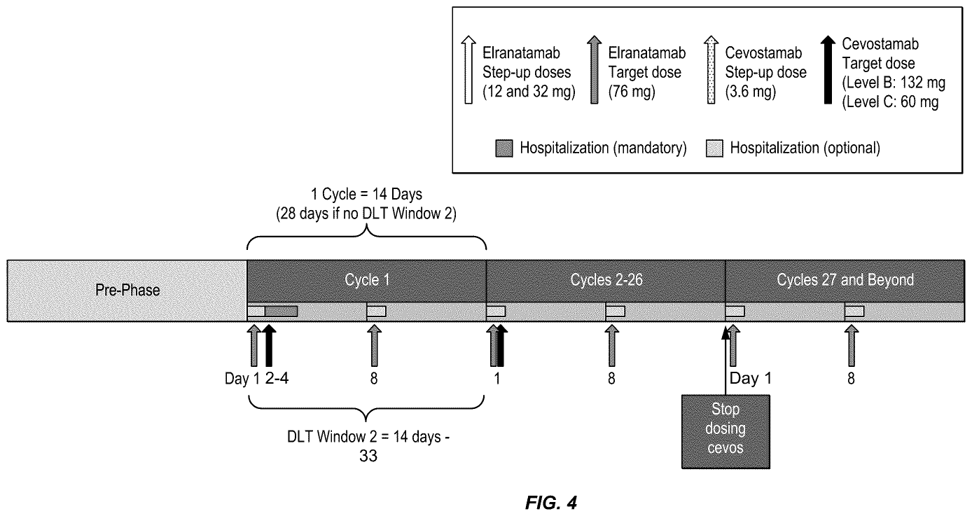

FIG. 5 is a schematic diagram showing the GO43979 dosing schedule for target dose level D (alternating dosing schedule) with target dose mitigation.

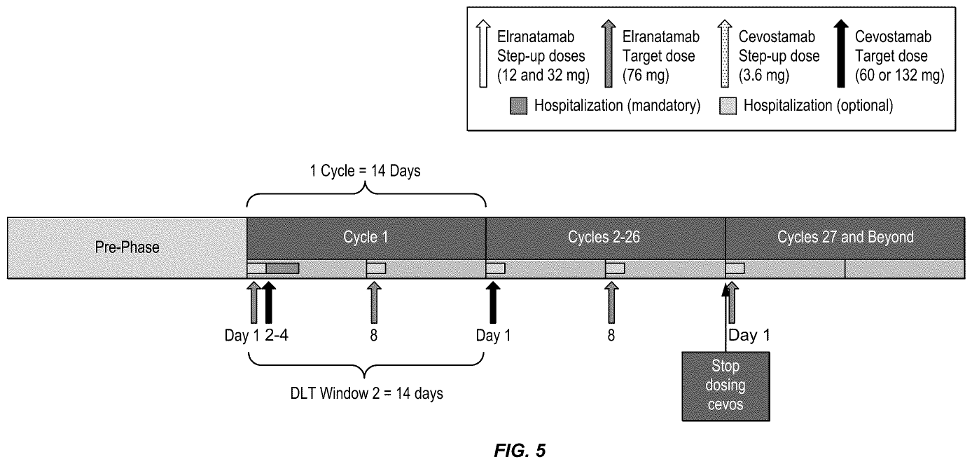

FIG. 6 is a schematic diagram showing GO43979 study treatment dosing schedule without cevostamab step-up dose (Option 3).

FIG. 7 is a schematic diagram showing the GO43979 dosing schedule for target dose levels B and C (FIG. 6).

FIG. 8 is a schematic diagram showing the GO43979 dosing schedule for target dose level D (alternating dosing schedule) with target dose mitigation.

FIG. 9 is a schematic diagram showing the GO43979 dosing schedule for target dose level E.

FIG. 10 is a schematic diagram showing the GO43979 dosing schedule for target dose levels F and G.

### DETAILED DESCRIPTION OF THE INVENTION

### I. Definitions

The term “about” as used herein refers to the usual error range for the respective value readily known to the skilled person in this technical field. Reference to “about” a value or parameter herein includes (and describes) aspects that are directed to that value or parameter per se.

It is understood that aspects of the invention described herein include “comprising,” “consisting,” and “consisting essentially of” aspects.

The term “FcRH5” or “fragment crystallizable receptor-like 5,” as used herein, refers to any native FcRH5 from any vertebrate source, including mammals such as primates (e.g., humans) and rodents (e.g., mice and rats), unless otherwise indicated, and encompasses “full-length,” unprocessed FcRH5, as well as any form of FcRH5 that results from processing in the cell. The term also encompasses naturally occurring variants of FcRH5, including, for example, splice variants or allelic variants. FcRH5 includes, for example, human FcRH5 protein (UniProtKB/Swiss-Prot ID: Q96RD9.3), which is 977 amino acids in length.

The terms “anti-FcRH5 antibody,” “an antibody that binds to FcRH5,” and “an antibody that specifically binds to FcRH5” refer to an antibody that is capable of binding FcRH5 with sufficient affinity such that the antibody is useful as a diagnostic and/or therapeutic agent in targeting FcRH5. In one embodiment, the extent of binding of an anti-FcRH5 antibody to an unrelated, non-FcRH5 protein is less than about 10% of the binding of the antibody to FcRH5 as measured, e.g., by a radioimmunoassay (RIA). In certain embodiments, an antibody that binds to FcRH5 has a dissociation constant (KD) of ≤1 μM, ≤250 nM, ≤100 nM, ≤15 nM, ≤10 nM, ≤6 nM, ≤4 nM, ≤2 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-FcRH5 antibody binds to an epitope of FcRH5 that is conserved among FcRH5 from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “anti-CD3 antibody,” “an antibody that binds to CD3,” and “an antibody that specifically binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (KD) of ≤1 μM, ≤250 nM, ≤100 nM, ≤15 nM, ≤10 nM, ≤5 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD3 antibody binds to an epitope of CD3 that is conserved among CD3 from different species.

For the purposes herein, “cevostamab,” also referred to as BFCR4350A or RO7187797, is an Fc-engineered, humanized, full-length non-glycosylated IgG1 kappa T-cell-dependent bispecific (TDB) antibody that specifically binds FcRH5 and CD3 and comprises an anti-FcRH5 arm comprising the heavy chain polypeptide sequence of SEQ ID NO: 35 and the light chain polypeptide sequence of SEQ ID NO: 36 and an anti-CD3 arm comprising the heavy chain polypeptide sequence of SEQ ID NO: 37 and the light chain polypeptide sequence of SEQ ID NO: 38. Cevostamab comprises a threonine to tryptophan amino acid substitution at position 366 on the heavy chain of the anti-FcRH5 arm (T366W) using EU numbering of Fc region amino acid residues and three amino acid substitutions (tyrosine to valine at position 407, threonine to serine at position 366, and leucine to alanine at position 368) on the heavy chain of the anti-CD3 arm (Y407V, T366S, and L368A) using EU numbering of Fc region amino acid residues to drive heterodimerization of the two arms (half-antibodies). Cevostamab also comprises an amino acid substitution (asparagine to glycine) at position 297 on each heavy chain (N297G) using EU numbering of Fc region amino acid residues, which results in a non-glycosylated antibody that has minimal binding to Fc (Fcγ) receptors and, consequently, prevents Fc-effector function. Cevostamab is also described in WHO Drug Information (International Nonproprietary Names for Pharmaceutical Substances), Recommended INN: List 84, Vol. 34, No. 3, published 2020 (see page 701).

The term “B-cell maturation antigen” or “BCMA,” as used herein, refers to any native BCMA from any vertebrate source, including mammals such as primates (e.g., humans) and rodents (e.g., mice and rats), unless otherwise indicated. BCMA is also known in the art as tumor necrosis factor superfamily member 17. The term encompasses “full-length,” unprocessed BCMA, as well as any form of BCMA that results from processing in the cell. The term also encompasses naturally occurring variants of BCMA, including, for example, splice variants or allelic variants. BCMA includes, for example, human BCMA protein (NCBI RefSeq No. NP 001183), which is 184 amino acids in length.

The terms “anti-BCMA antibody,” “an antibody that binds to BCMA,” and “an antibody that specifically binds to BCMA” refer to an antibody that is capable of binding BCMA with sufficient affinity such that the antibody is useful as a diagnostic and/or therapeutic agent in targeting BCMA. In one embodiment, the extent of binding of an anti-BCMA antibody to an unrelated, non-BCMA protein is less than about 10% of the binding of the antibody to BCMA as measured, e.g., by an RIA. In certain embodiments, an antibody that binds to BCMA has a KD of ≤1 μM, ≤250 nM, ≤100 nM, ≤15 nM, ≤10 nM, ≤6 nM, ≤4 nM, ≤2 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-BCMA antibody binds to an epitope of BCMA that is conserved among BCMA from different species.

For the purposes herein, “elranatamab,” also referred to as PF-06863135, is heterodimeric humanized full-length bispecific IgG2 kappa TDB antibody that specifically binds to BCMA and CD3. Targeted T-cell-mediated cytotoxicity follows the binding of one epitope of elranatamab to CD3-expressing T-cells and a second epitope to BCMA-expressing cells (e.g., MM cells). Elranatamab is described, e.g., in Chemical Abstract Services (CAS) Registry Number 2408850-14-4, United States Adopted Names (USAN) File Number (HI-199), and the WHO Drug Information ((International Nonproprietary Names for Pharmaceutical Substances), Recommended INN: List 87, Vol. 36, No. 1, published 2022, pages 100-103. In some examples, elranatamab comprises an anti-BCMA arm comprising the heavy chain polypeptide sequence of SEQ ID NO: 50 and the light chain polypeptide sequence of SEQ ID NO: 51 and an anti-CD3 arm comprising the heavy chain polypeptide sequence of SEQ ID NO: 63 and the light chain polypeptide sequence of SEQ ID NO: 64. In some examples, the heavy chain polypeptide sequence for the anti-BCMA arm of elranatamab may lack the C-terminal lysine residue that is present in SEQ ID NO: 50. In some examples, the heavy chain polypeptide sequence for the anti-CD3 arm of elranatamab may lack the C-terminal lysine residue that is present in SEQ ID NO: 63. In some examples, the heavy chain polypeptide sequence for the anti-BCMA arm of elranatamab may lack the C-terminal lysine residue that is present in SEQ ID NO: 50 and the heavy chain polypeptide sequence for the anti-CD3 arm of elranatamab may lack the C-terminal lysine residue that is present in SEQ ID NO: 63.

The term “antibody” herein is used in the broadest sense and encompasses various antibody structures, including but not limited to monoclonal antibodies, polyclonal antibodies, multispecific antibodies (e.g., bispecific antibodies (e.g., bispecific antibodies that specifically bind to FcRH5 and CD3 or bispecific antibodies that specifically bind to BCMA and CD3), and antibody fragments (e.g., bis-Fabs) so long as they exhibit the desired antigen-binding activity.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Specific illustrative and exemplary aspects for measuring binding affinity are described in the following.

With regard to the binding of an antibody to a target molecule, the term “binds” or “binding” or “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “full-length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to bis-Fabs; Fv; Fab; Fab, Fab′-SH; F(ab′)2; diabodies; linear antibodies; single-chain antibody molecules (e.g., scFv, ScFab); and multispecific antibodies formed from antibody fragments.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

A “Fab” fragment is an antigen-binding fragment generated by papain digestion of antibodies and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, preferably at least about 90% homology therewith, or preferably at least about 95% homology therewith.

“Fc complex” as used herein refers to CH3 domains of two Fc regions interacting together to form a dimer or, as in certain aspects, two Fc regions interact to form a dimer, wherein the cysteine residues in the hinge regions and/or the CH3 domains interact through bonds and/or forces (e.g., Van der Waals, hydrophobic forces, hydrogen bonds, electrostatic forces, or disulfide bonds).

“Fc component” as used herein refers to a hinge region, a CH2 domain or a CH3 domain of an Fc region.

“Hinge region” is generally defined as stretching from about residue 216 to 230 of an IgG (EU numbering), from about residue 226 to 243 of an IgG (Kabat numbering), or from about residue 1 to 15 of an IgG (IMGT unique numbering).

The “lower hinge region” of an Fc region is normally defined as the stretch of residues immediately C-terminal to the hinge region, i.e., residues 233 to 239 of the Fc region (EU numbering).

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and preferably at least about 90% homology therewith, more preferably at least about 95% homology therewith.

“Fc receptor” or “FcR” describes a receptor that binds to the Fc region of an antibody. A preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “knob-into-hole” or “KnH” technology as mentioned herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc interaction interfaces, CL:CH1 interfaces or VH/VL interfaces of antibodies (e.g., US2007/0178552, WO 96/027011, WO 98/050431 and Zhu et al. (1997) Protein Science 6:781-788). This is especially useful in driving the pairing of two different heavy chains together during the manufacture of multispecific antibodies. For example, multispecific antibodies having KnH in their Fc regions can further comprise single variable domains linked to each Fc region, or further comprise different heavy chain variable domains that pair with identical, similar, or different light chain variable domains. KnH technology can also be used to pair two different receptor extracellular domains together or any other polypeptide sequences that comprise different target recognition sequences.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-HT(L1)-FR2-H2(L2)-FR3-H3(L3)-FR4.

The “CH1 region” or “CH1 domain” comprises the stretch of residues from about residue 118 to residue 215 of an IgG (EU numbering), from about residue 114 to 223 of an IgG (Kabat numbering), or from about residue 1.4 to residue 121 of an IgG (IMGT unique numbering) (Lefranc M-P, Giudicelli V, Duroux P, Jabado-Michaloud J, Folch G, Aouinti S, Carillon E, Duvergey H, Houles A, Paysan-Lafosse T, Hadi-Saljoqi S, Sasorith S, Lefranc G, Kossida S. IMGT®, the international ImMunoGeneTics information system® 25 years on. Nucleic Acids Res. 2015 January; 43 (Database issue):D413-22).

The “CH2 domain” of a human IgG Fc region usually extends from about residues 244 to about 360 of an IgG (Kabat numbering), from about residues 231 to about 340 of an IgG (EU numbering), or from about residues 1.6 to about 125 of an IgG (IGMT unique numbering). The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22: 161-206 (1985).

The “CH3 domain” comprises the stretch of residues C-terminal to a CH2 domain in an Fc region (i.e., from about amino acid residue 361 to about amino acid residue 478 of an IgG (Kabat numbering), from about amino acid residue 341 to about amino acid residue 447 of an IgG (EU numbering), or from about amino acid residue 1.4 to about amino acid residue 130 of an IgG (IGMT unique numbering)).

The “CL domain” or “constant light domain” comprises the stretch of residues C-terminal to a light-chain variable domain (VL). The light chain of an antibody may be a kappa (κ) (“Cκ”) or lambda (λ) (“Cλ”) light chain region. The CK region generally extends from about residue 108 to residue 214 of an IgG (Kabat or EU numbering) or from about residue 1.4 to residue 126 of an IgG (IMGT unique numbering). The Ck residue generally extends from about residue 107a to residue 215 (Kabat numbering) or from about residue 1.5 to residue 127 (IMGT unique numbering) (Lefranc M-P, Giudicelli V, Duroux P, Jabado-Michaloud J, Folch G, Aouinti S, Carillon E, Duvergey H, Houles A, Paysan-Lafosse T, Hadi-Saljoqi S, Sasorith S, Lefranc G, Kossida S. IMGT®, the international ImMunoGeneTics information system® 25 years on. Nucleic Acids Res. 2015 January; 43 (Database issue):D413-22).

The light chain (LC) from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, γ, ε, and μ, respectively. The γ and α classes are further divided into subclasses on the basis of relatively minor differences in CH sequence and function, e.g., humans express the following subclasses: IgG1, IgG2, IgG3, IgG4, IgAQ1, and IgA2.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgAQ1, and IgA2. The heavy chain constant domains that correspond to the different classes of immunoglobulins are called α, δ, ε, γ, and μ, respectively.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter. J Mol. Biol. 227:381, 1991; Marks et al. J. Mol. Biol. 222:581, 1991. Also available for the preparation of human monoclonal antibodies are methods described in Cole et al. Monoclonal Antibodies and Cancer Therapy, Alan R. Liss, p. 77 (1985); Boerner et al. J. Immunol., 147(1):86-95, 1991. See also van Dijk and van de Winkel. Curr. Opin. Pharmacol. 5:368-74, 2001.

Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al. Proc. Natl. Acad. Sci. USA. 103:3557-3562, 2006 regarding human antibodies generated via a human B-cell hybridoma technology.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al. supra. In one aspect, for the VH, the subgroup is subgroup III as in Kabat et al. supra.

A “humanized” antibody refers to a chimeric antibody comprising amino acid residues from nonhuman HVRs and amino acid residues from human FRs. In certain aspects, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. In certain aspects in which all or substantially all of the FRs of a humanized antibody correspond to those of a human antibody, any of the FRs of the humanized antibody may contain one or more amino acid residues (e.g., one or more Vernier position residues of FRs) from non-human FR(s). A humanized antibody optionally may comprise at least a portion of an antibody constant region derived from a human antibody. A “humanized form” of an antibody, e.g., a non-human antibody, refers to an antibody that has undergone humanization.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed. W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al. J. Immunol. 150:880-887, 1993; Clarkson et al. Nature 352:624-628, 1991.

The term “hypervariable region” or “HVR” as used herein refers to each of the regions of an antibody variable domain which are hypervariable in sequence (“complementarity determining regions” or “CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:
(a) CDRs occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917, 1987);
(b) CDRs occurring at amino acid residues 24-34 (L1), 50-56 (L2), 89-97 (L3), 31-35b (H1), 5065 (H2), and 95-102 (H3) (Kabat et al. Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745, 1996).

Unless otherwise indicated, HVR residues and other residues in the variable domain (e.g., FR residues) are numbered herein according to Kabat et al. supra.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains, which enables the scFv to form the desired structure for antigen binding. For a review of scFv, see Pluckthun, The Pharmacology of Monoclonal Antibodies, vol. 113, Rosenburg and Moore eds., Springer-Verlag, New York, pp. 269-315 (1994); Malmborg et al., J. Immunol. Methods 183:7-13, 1995.

By “targeting domain” is meant a part of a compound or a molecule that specifically binds to a target epitope, antigen, ligand, or receptor. Targeting domains include but are not limited to antibodies (e.g., monoclonal, polyclonal, recombinant, humanized, and chimeric antibodies), antibody fragments or portions thereof (e.g., bis-Fab fragments, Fab fragments, F(ab′)2, scFab, scFv antibodies, SMIP, single-domain antibodies, diabodies, minibodies, scFv-Fc, affibodies, nanobodies, and VH and/or VL domains of antibodies), receptors, ligands, aptamers, peptide targeting domains (e.g., cysteine knot proteins (CKP)), and other molecules having an identified binding partner. A targeting domain may target, block, agonize, or antagonize the antigen to which it binds.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “multispecific antibody” is used in the broadest sense and specifically covers an antibody that has polyepitopic specificity. In one aspect, the multispecific antibody binds to two different targets (e.g., bispecific antibody). Such multispecific antibodies include, but are not limited to, an antibody comprising a heavy chain variable domain (VH) and a light chain variable domain (VL), where the VH/VL unit has polyepitopic specificity, antibodies having two or more VL and VH domains with each VH/VL unit binding to a different epitope, antibodies having two or more single variable domains with each single variable domain binding to a different epitope, full-length antibodies, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). “Monospecific” refers to the ability to bind only one antigen. In one aspect, the monospecific biepitopic antibody binds two different epitopes on the same target/antigen. In one aspect, the monospecific polyepitopic antibody binds to multiple different epitopes of the same target/antigen. According to one aspect, the multispecific antibody is an IgG antibody that binds to each epitope with an affinity of 5 μM to 0.001 pM, 3 μM to 0.001 pM, 1 μM to 0.001 pM, 0.5 μM to 0.001 pM, or 0.1 μM to 0.001 pM.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

A “disorder” is any condition that would benefit from treatment including, but not limited to, chronic and acute disorders or diseases including those pathological conditions which predispose a mammal to the disorder in question. In one aspect, the disorder is a cancer, e.g., a multiple myeloma (MM) (e.g., a relapsed or refractory (R/R) MM).

The terms “cell proliferative disorder” and “proliferative disorder” refer to disorders that are associated with some degree of abnormal cell proliferation. In one aspect, the cell proliferative disorder is cancer. In one aspect, the cell proliferative disorder is a tumor.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer” and “cancerous” refer to or describe the physiological condition in mammals that is typically characterized by unregulated cell growth/proliferation. Aspects of cancer include solid tumor cancers and non-solid tumor cancers. Examples of cancer include, but are not limited to, B cell proliferative disorders, such as MM, which may be R/R MM.

The term “B cell proliferative disorder” or “B cell malignancy” refers to a disorder that is associated with some degree of abnormal B cell proliferation.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

A “functional Fc region” possesses an effector function of a native sequence Fc region. Such effector functions generally require the Fc region to be combined with a binding domain (e.g., an antibody variable domain) and can be assessed using various assays as disclosed, for example, in definitions herein.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) that are bound to their cognate antigen. To assess complement activation, a CDC assay, e.g., as described in Gazzano-Santoro et al., J. Immunol. Methods 202:163 (1996), can be performed.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxic agents. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet. Annu. Rev. Immunol. 9:457-92, 1991. To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. Proc. Natl. Acad. Sci. USA. 95:652-656, 1998.

“Complex” or “complexed” as used herein refers to the association of two or more molecules that interact with each other through bonds and/or forces (e.g., Van der Waals, hydrophobic, hydrophilic forces) that are not peptide bonds. In one aspect, the complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

As used herein, “delaying progression” of a disorder or disease means to defer, hinder, slow, retard, stabilize, and/or postpone development of the disease or disorder (e.g., a cell proliferative disorder, e.g., cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

An “effective amount” of a compound, for example, an anti-FcRH5/anti-CD3 bispecific antibody (e.g., cevostamab) and/or an anti-BCMA/anti-CD3 bispecific antibody (e.g., elranatamab) of the disclosure or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “objective response rate” (ORR) refers to the proportion of patients with a stringent complete response (sCR), complete response (CR), very good partial response (VGPR), or partial response on two consecutive occasions, as determined using the International Myeloma Working Group response criteria.

As used herein, “complete response” or “CR” refers to a response characterized by no evidence of initial monoclonal protein isotype(s) on immunofixation of the serum and urine, disappearance of any soft tissue plasmacytomas, and ≤5% plasma cells in bone marrow (BM). “Stringent complete response” or “sCR” refers to a CR as defined above, plus a normal free light-chain (FLC) ratio and absence of clonal cells in BM by immunohistochemistry (kappa/lambda ratio ≤4:1 or ≥1:2 for kappa and lambda patients, respectively, after counting ≥100 plasma cells). In some examples, CR/sCR rate is defined as proportion of patients achieving a CR or sCR on two consecutive occasions.

As used herein, “very good partial response” or “VGPR” refers to a response characterized by serum and urine M-protein detectable by immunofixation but not on electrophoresis; or ≥90% reduction in serum M protein plus urine M-protein level <100 mg/24 hours. In some examples, rate of VGPR or better is defined as the proportion of patients achieving a VGPR or better on two consecutive occasions.

As used herein, “partial response” or “PR” refers to a response characterized by ≥50% reduction of serum M-protein and reduction in 24-hour urine M protein by ≥90% or to <200 mg/24 hours. If the serum and urine M-protein are unmeasurable, a ≥50% decrease in the difference between involved and uninvolved FLC levels may be determined in place of the M-protein criteria. If serum and urine M-protein are unmeasurable and serum FLC assay is also unmeasurable, a ≥50% reduction in plasma cells may be determined in place of M-protein, provided baseline BM plasma cell percentage was ≥30%. In addition, if soft tissue plasmacytomas were present at baseline, a partial response comprises a ≥50% reduction in the size (SPD) of soft tissue plasmacytomas.

As used herein, “progression-free survival” (PFS) refers to the length of time during and after treatment during which the disease being treated (e.g., cancer) does not get worse. Progression-free survival may include the amount of time patients have experienced a CR or a PR, as well as the amount of time patients have experienced stable disease. In some examples, PFS is defined as the time from initiation of study treatment to the first date of disease progression or death from any cause, whichever occurs first.

As used herein, “duration of response” (DOR) is defined as the time from the first occurrence of a documented objective response to disease progression or death from any cause, whichever occurs first.

In some examples, DOR is defined as the number of days from the date of first documented response of PR or better until date of disease progression or death from any cause, whichever occurs first.

As used herein, “survival” refers to the patient remaining alive, and includes overall survival as well as progression-free survival.

As used herein, “overall survival” and “OS” refer to the length of time from either the date of diagnosis or the start of treatment for a disease (e.g., cancer) that the patient is still alive. For example, OS may be defined as the time from initiation of study treatment to death from any cause.

The term “epitope” refers to the particular site on an antigen molecule to which an antibody binds. In some aspects, the particular site on an antigen molecule to which an antibody binds is determined by hydroxyl radical footprinting. In some aspects, the particular site on an antigen molecule to which an antibody binds is determined by crystallography.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject or individual is a human.

An “isolated” protein or peptide is one which has been separated from a component of its natural environment. In some aspects, a protein or peptide is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the disclosure (e.g., anti-FcRH5/anti-CD3 TDBs and/or an anti-BCMA/anti-CD3 TDBs of the disclosure) are used to delay development of a disease or to slow the progression of a disease.

By “reduce” or “inhibit” is meant the ability to cause an overall decrease, for example, of 20% or greater, of 50% or greater, or of 75%, 85%, 90%, 95%, or greater. In certain aspects, reduce or inhibit can refer to the effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP).

As used herein, “administering” is meant a method of giving a dosage of a compound (e.g., an anti-FcRH5/anti-CD3 bispecific antibody (e.g., cevostamab) and/or an anti-BCMA/anti-CD3 bispecific antibody (e.g., elranatamab) of the disclosure) to a subject. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some aspects, the compositions utilized in the methods herein are administered intravenously. In some aspects, the compositions utilized in the methods herein are administered subcutaneously. The method of administration can vary depending on various factors (e.g., the compound or composition being administered and the severity of the condition, disease, or disorder being treated).

### II. Therapeutic Methods and Compositions for Use

The present application is based, in part, on methods of treating a subject having cancer (e.g., multiple myeloma (MM) (e.g., R/R MM)) with an anti-fragment crystallizable receptor-like 5 (FcRH5)/anti-cluster of differentiation 3 (CD3) bispecific antibody (e.g., cevostamab) in combination with an anti-B-cell maturation antigen (BCMA)/anti-CD3 bispecific antibody, e.g., using fractionated, dose-escalation dosing regimens as disclosed herein. The anti-FcRH5/anti-CD3 and anti-BCMA/anti-CD3 combinatorial dosing regimens described herein are expected to improve and deepen therapeutic responses, thereby increasing survival while maintaining quality of life in cancer subjects.

### A. Dosing Regimens

i. Dosing Regimens for Treatment of a Cancer

The present disclosure provides methods and compositions for treatment of a cancer (e.g., a hematologic cancer (e.g., a B cell proliferative disorder (e.g., an MM))).

For example, provided herein is a method of treating a subject having a cancer (e.g., a hematologic cancer (e.g., a B cell proliferative disorder (e.g., an MM))), the method comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab).

In another example, provided herein is a bispecific antibody that binds to FcRH5 and CD3 for use in treatment of a subject having a cancer (e.g., a hematologic cancer (e.g., a B cell proliferative disorder (e.g., an MM))), the treatment comprising administration of an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) to the subject.

In another example, provided herein is a bispecific antibody that binds to BCMA and CD3 for use in treatment of a subject having a cancer (e.g., a hematologic cancer (e.g., a B cell proliferative disorder (e.g., an MM))), the treatment comprising administration of an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) to the subject.

In some examples, the subject has relapsed or refractory (R/R) MM. In some examples, the subject has diagnosis of R/R MM according to International Myeloma Working Group (IMWG) criteria.

The first bispecific antibody (e.g., an anti-FcRH5/anti-CD3 antibody, e.g., cevostamab) and second bispecific antibody (e.g., an anti-BCMA/anti-CD3 antibody, e.g., elranatamab) is administered to the subject in a dosing regimen comprising a first phase comprising one or more dosing cycles, a second phase comprising one or more dosing cycles, and, optionally, a third phase comprising one or more dosing cycles. In some embodiments, the dosing regimen comprises the first phase and the second phase but does not comprise the third phase. In other embodiments, the dosing regimen comprises the first phase, the second phase, and the third phase. Each dosing cycle of the first phase, second phase, and/or third phase may have any suitable length. In some examples, each dosing cycle of the first phase, the second phase, and/or the third phase is a 14-day dosing cycle. In some examples, the dosing cycle of the second phase or the third phase is a 28-day dosing cycle.

With regard to the first phase, the first bispecific antibody (e.g., an anti-FcRH5/anti-CD3 antibody, e.g., cevostamab) may be administered in a dosing regimen comprising administering the first bispecific antibody to the subject on Day 2, Day 3, or Day 4 of a dosing cycle having any suitable length described herein. In some examples, each dosing cycle of the first phase is a 14-day dosing cycle. In some examples, the first phase comprises or consists of a single 14-day dosing cycle (C1). In some examples, the first phase consists of a single 14-day dosing cycle (C1). In a particular example, the first bispecific antibody (e.g., an anti-FcRH5/anti-CD3 antibody, e.g., cevostamab) is administered to the subject on Day 2 of a 14-day dosing cycle. In another example, the first bispecific antibody (e.g., an anti-FcRH5/anti-CD3 antibody, e.g., cevostamab) is administered to the subject on Day 3 of a 14-day dosing cycle. In yet another example, the first bispecific antibody (e.g., an anti-FcRH5/anti-CD3 antibody, e.g., cevostamab) is administered to the subject on Day 4 of a 14-day dosing cycle.

With continued regard to the first phase, the second bispecific antibody (e.g., an anti-BCMA/anti-CD3 antibody, e.g., elranatamab) may be administered in a dosing regimen comprising administering the second bispecific antibody to the subject on Day 1 and/or Day 8 of a dosing cycle having any suitable length described herein. In some examples, each dosing cycle of the first phase is a 14-day dosing cycle. In some examples, each dosing cycle of the first phase is a 14-day dosing cycle. In some examples, the first phase comprises or consists of a single 14-day dosing cycle (C1). In some examples, the first phase consists of a single 14-day dosing cycle (C1). In a particular example, the second bispecific antibody (e.g., an anti-BCMA/anti-CD3 antibody, e.g., elranatamab) is administered to the subject on Day 1 of a 14-day dosing cycle. In another example, the second bispecific antibody (e.g., an anti-BCMA/anti-CD3 antibody, e.g., elranatamab) is administered to the subject on Day 8 of a 14-day dosing cycle. In yet another example, the second bispecific antibody (e.g., an anti-BCMA/anti-CD3 antibody, e.g., elranatamab) is administered to the subject on Day 1 and Day 8 of a 14-day dosing cycle.

With regard to the second phase, the first bispecific antibody (e.g., an anti-FcRH5/anti-CD3 antibody, e.g., cevostamab) may administered in a dosing regimen comprising administering the first bispecific antibody to the subject on Day 1 of a dosing cycle having any suitable length described herein. In some embodiments, each dosing cycle of the first second is a 14-day dosing cycle. In a particular example, the first bispecific antibody (e.g., an anti-FcRH5/anti-CD3 antibody, e.g., cevostamab) is administered to the subject on Day 1 of a 14-day dosing cycle.

With continued regard to the second phase, the second bispecific antibody (e.g., an anti-BCMA/anti-CD3 antibody, e.g., elranatamab) may be administered in a dosing regimen comprising administering the second bispecific antibody to the subject on Day 1 and/or Day 8 of a dosing cycle having any suitable length described herein. In some embodiments, each dosing cycle of the second phase is a 14-day dosing cycle. In a particular example, the second bispecific antibody (e.g., an anti-BCMA/anti-CD3 antibody, e.g., elranatamab) is administered to the subject on Day 1 of a 14-day dosing cycle. In another example, the second bispecific antibody (e.g., an anti-BCMA/anti-CD3 antibody, e.g., elranatamab) is administered to the subject on Day 8 of a 14-day dosing cycle. In yet another example, the second bispecific antibody (e.g., an anti-BCMA/anti-CD3 antibody, e.g., elranatamab) is administered to the subject on Day 1 and Day 8 of a 14-day dosing cycle.

The first phase and the second phase may include any suitable number of dosing cycles, e.g., one, two, three, four, five, six, seven, eight, nine, ten, eleven, twelve, thirteen, fourteen, fifteen, sixteen, seventeen, or more dosing cycles. In some embodiments, the first phase and/or the second phase last until disease progression or unacceptable toxicity, or up to 1 year on treatment.

With regard to the optional third phase of the dosing regimen, in some examples, the first bispecific antibody (e.g., an anti-FcRH5/anti-CD3 antibody, e.g., cevostamab) is not administered.

With continued regard to the optional third phase, the second bispecific antibody (e.g., an anti-BCMA/anti-CD3 antibody, e.g., elranatamab) may be administered in a dosing regimen comprising administering the second bispecific antibody to the subject on Day 1 and/or Day 8 of a dosing cycle having any suitable length described herein. In some embodiments, each dosing cycle of the third phase is a 14-day dosing cycle. In a particular example, the second bispecific antibody (e.g., an anti-BCMA/anti-CD3 antibody, e.g., elranatamab) is administered to the subject on Day 1 of a 14-day dosing cycle. In another example, the second bispecific antibody (e.g., an anti-BCMA/anti-CD3 antibody, e.g., elranatamab) is administered to the subject on Day 1 and Day 8 of a 14-day dosing cycle.

The method of treatment may further comprise a pre-phase, prior to the first phase, comprising one or more dosing cycles, wherein the pre-phase comprises administering the first bispecific antibody (e.g., an anti-FcRH5/anti-CD3 antibody, e.g., cevostamab) and the second bispecific antibody (e.g., an anti-BCMA/anti-CD3 antibody, e.g., elranatamab) to the subject in a dosing regimen described herein.

Each dosing cycle of the pre-phase may have any suitable length. In some examples, each dosing cycle of the pre-phase is an approximately 15-day dosing cycle. In some examples, the pre-phase comprises or consists of a single 15-day dosing cycle. In some examples, the pre-phase consists of a single 15-day dosing cycle. In some examples, each dosing cycle of the pre-phase is an approximately 22-day dosing cycle. In some examples, the pre-phase comprises or consists of a single 15-day dosing cycle. In some examples, the pre-phase consists of a single 22-day dosing cycle. In some examples, the pre-phase continues until any CRS events experienced in the pre-phase are resolved. In some examples, the pre-phase lasts until 7 days after the last dose of the first bispecific antibody (e.g., cevostamab) in the pre-phase.

In some examples, the method of treatment comprises a pre-phase, prior to the first phase, comprising one or more dosing cycles, wherein the pre-phase comprises administering the first bispecific antibody (e.g., an anti-FcRH5/anti-CD3 antibody, e.g., cevostamab) to the subject on Day 9, Day 10, or Day 11 of a dosing cycle having any suitable length described herein. In some examples, the pre-phase comprises or consists of approximately 15 days. In one particular example, the first bispecific antibody (e.g., an anti-FcRH5/anti-CD3 antibody, e.g., cevostamab) is administered to the subject on Day 9 of an approximately 15-day dosing cycle in the pre-phase. In another example, the first bispecific antibody (e.g., an anti-FcRH5/anti-CD3 antibody, e.g., cevostamab) is administered on Day 10 of an approximately 15-day dosing cycle in the pre-phase. In yet another example, the first bispecific antibody (e.g., an anti-FcRH5/anti-CD3 antibody, e.g., cevostamab) is administered on Day 11 of an approximately 15-day dosing cycle in the pre-phase. The pre-phase can further comprise administering the second bispecific antibody (e.g., an anti-BCMA/anti-CD3 antibody, e.g., elranatamab) to the subject. In one particular example, the second bispecific antibody (e.g., an anti-BCMA/anti-CD3 antibody, e.g., elranatamab) is administered to the subject on Day 1, Day 3, and Day 8 of an approximately 15-day dosing cycle in the pre-phase. In another example, the second bispecific antibody (e.g., an anti-BCMA/anti-CD3 antibody, e.g., elranatamab) is administered on Day 1, Day 4, and Day 8 of an approximately 15-day dosing cycle in the pre-phase. In yet another example, the second bispecific antibody (e.g., an anti-BCMA/anti-CD3 antibody, e.g., elranatamab) is administered on Day 1, Day 5, and Day 8 of an approximately 15-day dosing cycle in the pre-phase.

In other examples, the method of treatment comprises a pre-phase, prior to the first phase, comprising one or more dosing cycles, wherein the pre-phase comprises administering the first bispecific antibody (e.g., an anti-FcRH5/anti-CD3 antibody, e.g., cevostamab) to the subject on Day 16, Day 17, or Day 18 of a dosing cycle having any suitable length described herein. In some examples, the pre-phase comprises or consists of approximately 22 days. In one particular example, the first bispecific antibody (e.g., an anti-FcRH5/anti-CD3 antibody, e.g., cevostamab) is administered on Day 16 of an approximately 22-day dosing cycle in the pre-phase. In another example, the first bispecific antibody (e.g., an anti-FcRH5/anti-CD3 antibody, e.g., cevostamab) is administered on Day 17 of an approximately 22-day dosing cycle in the pre-phase. In yet another example, the first bispecific antibody (e.g., an anti-FcRH5/anti-CD3 antibody, e.g., cevostamab) is administered on Day 18 of an approximately 22-day dosing cycle in the pre-phase. The pre-phase can further comprise administering the second bispecific antibody (e.g., an anti-BCMA/anti-CD3 antibody, e.g., elranatamab) to the subject. In one particular example, the second bispecific antibody (e.g., an anti-BCMA/anti-CD3 antibody, e.g., elranatamab) is administered to the subject on Day 1, Day 3, Day 8, and Day 15 of an approximately 22-day dosing cycle in the pre-phase. In another example, the second bispecific antibody (e.g., an anti-BCMA/anti-CD3 antibody, e.g., elranatamab) is administered on Day 1, Day 4, Day 8, and Day 15 of an approximately 22-day dosing cycle in the pre-phase. In yet another example, the second bispecific antibody (e.g., an anti-BCMA/anti-CD3 antibody, e.g., elranatamab) is administered on Day 1, Day 5, Day 8, and Day 15 of an approximately 22-day dosing cycle in the pre-phase.

The pre-phase may include any suitable number of dosing cycles, e.g., one, two, three, four, five, six, seven, eight, nine, ten, eleven, twelve, thirteen, fourteen, fifteen, sixteen, seventeen, or more dosing cycles.

In a particular example, the pre-phase comprises one dosing cycle (C1).

In some examples, a target dose of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered to the subject for each administration in the pre-phase. In other words, the pre-phase may not utilize step-up dosing for the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab).

In other examples, the pre-phase comprises administering a step-up dose of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) to the subject. The step-up dose of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) may be administered to the subject on any suitable day(s) of a dosing cycle (e.g., the C1).

For example, the step-up dose of the first bispecific antibody (e.g., an anti-FcRH5/anti-CD3 antibody, e.g., cevostamab) during the pre-phase may be between about 0.5 mg to about 19.9 mg (e.g., between about 1 mg to about 18 mg, between about 2 mg to about 15 mg, between about 3 mg to about 10 mg, between about 3.3 mg to about 6 mg, or between about 3.4 mg to about 4 mg, e.g., about 3 mg, 3.2 mg, 3.3 mg, 3.4 mg, 3.6 mg, 3.8 mg, 4 mg, 4.2 mg, 4.4 mg, 4.6 mg, 4.8 mg, 5 mg, 5.2 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, 8 mg, 8.2 mg, 8.4 mg, 8.6 mg, 8.8 mg, 9 mg, 9.2 mg, 9.4 mg, 9.6 mg, 9.8 mg, 10 mg, 10.2 mg, 10.4 mg, 10.6 mg, 10.8 mg, 11 mg, 11.2 mg, 11.4 mg, 11.6 mg, 11.8 mg, 12 mg, 12.2 mg, 12.4 mg, 12.6 mg, 12.8 mg, 13 mg, 13.2 mg, 13.4 mg, 13.6 mg, 13.8 mg, 14 mg, 14.2 mg, 14.4 mg, 14.6 mg, 14.8 mg, 15 mg, 15.2 mg, 15.4 mg, 15.6 mg, 15.8 mg, 16 mg, 16.2 mg, 16.4 mg, 16.6 mg, 16.8 mg, 17 mg, 18.2 mg, 18.4 mg, 18.6 mg, 18.8 mg, 19 mg, 19.2 mg, 19.4 mg, 19.6 mg, or 19.8 mg). In some examples, the step-up dose of the first bispecific antibody (e.g., an anti-FcRH5/anti-CD3 antibody, e.g., cevostamab) during the pre-phase is about 3.6 mg.

During the pre-phase (e.g., an approximately 15-day pre-phase), the step-up dose of the first bispecific antibody (e.g., an anti-FcRH5/anti-CD3 antibody, e.g., cevostamab) may be administered to the subject on Day 9, Day 10, or Day 11 of the C1. In a particular example, the step-up dose of the first bispecific antibody (e.g., an anti-FcRH5/anti-CD3 antibody, e.g., cevostamab) is administered to the subject on Day 9 of the C1. In another example, the step-up dose of the first bispecific antibody (e.g., an anti-FcRH5/anti-CD3 antibody, e.g., cevostamab) is administered to the subject on Day 10 of the C1. In yet another example, the step-up dose of the first bispecific antibody (e.g., an anti-FcRH5/anti-CD3 antibody, e.g., cevostamab) is administered to the subject on Day 11 of the C1.

During the pre-phase (e.g., an approximately 22-day pre-phase), the step-up dose of the first bispecific antibody (e.g., an anti-FcRH5/anti-CD3 antibody, e.g., cevostamab) may be administered to the subject on Day 16, Day 17, or Day 18 of the C1. In a particular example, the step-up dose of the first bispecific antibody (e.g., an anti-FcRH5/anti-CD3 antibody, e.g., cevostamab) is administered to the subject on Day 16 of the C1. In another example, the step-up dose of the first bispecific antibody (e.g., an anti-FcRH5/anti-CD3 antibody, e.g., cevostamab) is administered to the subject on Day 17 of the C1.

In yet another example, the step-up dose of the first bispecific antibody (e.g., an anti-FcRH5/anti-CD3 antibody, e.g., cevostamab) is administered to the subject on Day 18 of the C1.

In some examples, the pre-phase comprises administering a first step-up dose and a second step-up dose of the second bispecific antibody (e.g., an anti-BCMA/anti-CD3 antibody, e.g., elranatamab) to the subject.

The first step-up dose and/or the second step-up dose of the second bispecific antibody (e.g., an anti-BCMA/anti-CD3 antibody, e.g., elranatamab) may be administered to the subject on any suitable day of a dosing cycle (e.g., the C1) of the pre-phase.

For example, during the pre-phase, the first step-up dose of the second bispecific antibody (e.g., an anti-BCMA/anti-CD3 antibody, e.g., elranatamab) is administered to the subject on Day 1 of C1 and the second step-up dose is administered to the subject on Day 3 of the C1. In another example, the first step-up dose of the second bispecific antibody (e.g., an anti-BCMA/anti-CD3 antibody, e.g., elranatamab) is administered to the subject on Day 1 of C1 and the second step-up dose of the second bispecific antibody (e.g., an anti-BCMA/anti-CD3 antibody, e.g., elranatamab) is administered to the subject on Day 4 of the C1. In yet another example, the first step-up dose of the second bispecific antibody (e.g., an anti-BCMA/anti-CD3 antibody, e.g., elranatamab) is administered to the subject on Day 1 of C1 and the second step-up dose of the second bispecific antibody (e.g., an anti-BCMA/anti-CD3 antibody, e.g., elranatamab) is administered to the subject on Day 5 of the C1.

In a double step-up dosing regimen, one or more target doses of the second bispecific antibody (e.g., an anti-BCMA/anti-CD3 antibody, e.g., elranatamab) may be administered on any suitable day following the second step-up dose. In one particular example, a target dose of the second bispecific antibody (e.g., an anti-BCMA/anti-CD3 antibody, e.g., elranatamab) is administered to the subject on Day 8 of the C1. In another example, a target dose of the second bispecific antibody (e.g., an anti-BCMA/anti-CD3 antibody, e.g., elranatamab) is administered to the subject on Day 8 and Day 15 of the C1.

Any suitable dose may be used for the first step-up dose and second step-up dose. For example, the first step-up dose is 12 mg and the second step-up dose is 32 mg.

The first phase may comprise any suitable number of dosing cycles (e.g., 1, 2, 3, or more dosing cycles). In some examples, the first phase comprises one dosing cycle (C1). In some examples, the first phase consists of one dosing cycle (C1). In another example, the first phase comprises a first dosing cycle (C1) and a second dosing cycle (C2). In yet another example, the first phase comprises a first dosing cycle (C1), a second dosing cycle (C2), and a third dosing cycle (C3).

During the first phase, the method of treatment may comprise administering the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) to the subject. In a particular example, the first phase comprises administering the first bispecific antibody (e.g., an anti-FcRH5/anti-CD3 antibody, e.g., cevostamab) to the subject on Day 2, Day 3, or Day 4 of the C1 and the second bispecific antibody (e.g., an anti-BCMA/anti-CD3 antibody, e.g., elranatamab) to the subject on Day 1 and/or Day 8 of the C1. In another example, the first phase comprises administering the first bispecific antibody (e.g., an anti-FcRH5/anti-CD3 antibody, e.g., cevostamab) to the subject on Day 2, Day 3, or Day 4 of the C1 and C2, and the second bispecific antibody (e.g., an anti-BCMA/anti-CD3 antibody, e.g., elranatamab) to the subject on Day 1 and/or Day 8 of the C1 and C2. In yet another example, the first phase comprises administering the first bispecific antibody (e.g., an anti-FcRH5/anti-CD3 antibody, e.g., cevostamab) to the subject on Day 2, Day 3, or Day 4 of the C1, C2, and C3, and the second bispecific antibody (e.g., an anti-BCMA/anti-CD3 antibody, e.g., elranatamab) to the subject on Day 1 and/or Day 8 of the C1, C2, and C3.

In some examples, the first phase comprises administering the first bispecific antibody (e.g., an anti-FcRH5/anti-CD3 antibody, e.g., cevostamab) to the subject on Day 2 of the C1 and the second bispecific antibody (e.g., an anti-BCMA/anti-CD3 antibody, e.g., elranatamab) to the subject on Day 1 of the C1. In some examples, the first phase comprises administering the first bispecific antibody (e.g., an anti-FcRH5/anti-CD3 antibody, e.g., cevostamab) to the subject on Day 3 of the C1 and the second bispecific antibody (e.g., an anti-BCMA/anti-CD3 antibody, e.g., elranatamab) to the subject on Day 1 of the C1. In some examples, the first phase comprises administering the first bispecific antibody (e.g., an anti-FcRH5/anti-CD3 antibody, e.g., cevostamab) to the subject on Day 4 of the C1 and the second bispecific antibody (e.g., an anti-BCMA/anti-CD3 antibody, e.g., elranatamab) to the subject on Day 1 of the C1.

In some examples, the first phase comprises administering the first bispecific antibody (e.g., an anti-FcRH5/anti-CD3 antibody, e.g., cevostamab) to the subject on Day 2 of the C1 and the second bispecific antibody (e.g., an anti-BCMA/anti-CD3 antibody, e.g., elranatamab) to the subject on Day 1 and Day 8 of the C1. In some examples, the first phase comprises administering the first bispecific antibody (e.g., an anti-FcRH5/anti-CD3 antibody, e.g., cevostamab) to the subject on Day 3 of the C1 and the second bispecific antibody (e.g., an anti-BCMA/anti-CD3 antibody, e.g., elranatamab) to the subject on Day 1 and Day 8 of the C1. In some examples, the first phase comprises administering the first bispecific antibody (e.g., an anti-FcRH5/anti-CD3 antibody, e.g., cevostamab) to the subject on Day 4 of the C1 and the second bispecific antibody (e.g., an anti-BCMA/anti-CD3 antibody, e.g., elranatamab) to the subject on Day 1 and Day 8 of the C1.

The second phase may comprise any suitable number of dosing cycles (e.g., 1, 2, 3, 4, 5, 6, 7, 8, 9, 10, 11, 12, 13, 14, 15, 16, 17, 18, 19, 20, 21, 22, 23, 24, 25, or more dosing cycles). In some examples, the second phase comprises a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-forth dosing cycle (C24), and a twenty-fifth dosing cycle (C25).

In some examples, the second phase comprises administering the first bispecific antibody (e.g., an anti-FcRH5/anti-CD3 antibody, e.g., cevostamab) to the subject on Day 1 of the C1 and the second bispecific antibody (e.g., an anti-BCMA/anti-CD3 antibody, e.g., elranatamab) to the subject on Day 1 of the C1. The second phase may further comprise administering the first bispecific antibody (e.g., an anti-FcRH5/anti-CD3 antibody, e.g., cevostamab) to the subject on Day 1 of the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and the second bispecific antibody (e.g., an anti-BCMA/anti-CD3 antibody, e.g., elranatamab) to the subject on Day 1 of the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase.

In some examples, the second phase comprises administering the first bispecific antibody (e.g., an anti-FcRH5/anti-CD3 antibody, e.g., cevostamab) to the subject on Day 1 of the C1 and the second bispecific antibody (e.g., an anti-BCMA/anti-CD3 antibody, e.g., elranatamab) to the subject on Day 1 and Day 8 of the C1. In other words, in a 14-day dosing cycle, the second phase may comprise administering the first bispecific antibody (e.g., an anti-FcRH5/anti-CD3 antibody, e.g., cevostamab) every two weeks (Q2W) and the second bispecific antibody (e.g., an anti-BCMA/anti-CD3 antibody, e.g., elranatamab) every week (QW). In some examples, the second phase comprises administering the first bispecific antibody (e.g., an anti-FcRH5/anti-CD3 antibody, e.g., cevostamab) to the subject on Day 1 of the C1 and the second bispecific antibody (e.g., an anti-BCMA/anti-CD3 antibody, e.g., elranatamab) to the subject on Day 8 of the C1. In other words, in a 14-day dosing cycle, the second phase may comprise administering the first bispecific antibody (e.g., an anti-FcRH5/anti-CD3 antibody, e.g., cevostamab) Q2W and the second bispecific antibody (e.g., an anti-BCMA/anti-CD3 antibody, e.g., elranatamab) Q2W. The second phase may further comprise administering the first bispecific antibody (e.g., an anti-FcRH5/anti-CD3 antibody, e.g., cevostamab) to the subject on Day 1 of the C2, the C3, the C4, the C5, the C6, the C7, the C8, the C9, the C10, the C1, the C12, the C13, the C14, the C15, the C16, the C17, the C18, the C19, the C20, the C21, the C22, the C23, the C24, and the C25 of the second phase; and the second bispecific antibody (e.g., an anti-BCMA/anti-CD3 antibody, e.g., elranatamab) to the subject on Day 1 and Day 8 of the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase. In other words, the second phase may further comprise administering the first bispecific antibody (e.g., an anti-FcRH5/anti-CD3 antibody, e.g., cevostamab) Q2W and the second bispecific antibody (e.g., an anti-BCMA/anti-CD3 antibody, e.g., elranatamab) QW.

Alternatively, the second phase may further comprise administering the first bispecific antibody (e.g., an anti-FcRH5/anti-CD3 antibody, e.g., cevostamab) to the subject on Day 1 of the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and the second bispecific antibody (e.g., an anti-BCMA/anti-CD3 antibody, e.g., elranatamab) to the subject on Day 8 of the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase. In other words, the second phase may further comprise administering the first bispecific antibody (e.g., an anti-FcRH5/anti-CD3 antibody, e.g., cevostamab) Q2W and the second bispecific antibody (e.g., an anti-BCMA/anti-CD3 antibody, e.g., elranatamab) Q2W. In certain embodiments, the second phase, e.g., after achieving a complete response in the first 6 months of the first phase of administration, may further comprise administering the first bispecific antibody (e.g., an anti-FcRH5/anti-CD3 antibody, e.g., cevostamab) Q4W rather than Q2W and the second bispecific antibody (e.g., an anti-BCMA/anti-CD3 antibody, e.g., elranatamab) Q4W rather than Q2W. In certain embodiments, the second phase, e.g., after achieving at partial response for at least two months of the first phase, may further comprise administering the second bispecific antibody (e.g., an anti-BCMA/anti-CD3 antibody, e.g., elranatamab) Q4W rather than Q2W.

The third phase may comprise any suitable number of dosing cycles (e.g., 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 110, 120, 130, or more dosing cycles). In some examples, the third phase comprises a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-forth dosing cycle (C24), and a twenty-fifth dosing cycle (C25).

In some examples, the third phase comprises administering the second bispecific antibody (e.g., an anti-BCMA/anti-CD3 antibody, e.g., elranatamab) to the subject on Day 1 of the C1. The third phase may further comprise administering the second bispecific antibody (e.g., an anti-BCMA/anti-CD3 antibody, e.g., elranatamab) to the subject on Day 1 of the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the third phase. In other words, the third phase may further comprise administering the second bispecific antibody (e.g., an anti-BCMA/anti-CD3 antibody, e.g., elranatamab) every two weeks (Q2W) to the subject on Day 1 of each cycle in the third phase. In certain embodiments, the third phase, e.g., after achieving a complete response in the first 6 months of the second phase of administration, may further comprise administering the first bispecific antibody (e.g., an anti-FcRH5/anti-CD3 antibody, e.g., cevostamab) Q4W rather than Q2W and the second bispecific antibody (e.g., an anti-BCMA/anti-CD3 antibody, e.g., elranatamab) Q4W rather than Q2W. In certain embodiments, the third phase, e.g., after achieving at partial response for at least two months of the second phase, may further comprise administering the second bispecific antibody (e.g., an anti-BCMA/anti-CD3 antibody, e.g., elranatamab) Q4W rather than Q2W.

In some examples, the third phase comprises administering the second bispecific antibody (e.g., an anti-BCMA/anti-CD3 antibody, e.g., elranatamab) to the subject on Day 1 and/or Day 8 of the C1. The third phase may further comprise administering the second bispecific antibody (e.g., an anti-BCMA/anti-CD3 antibody, e.g., elranatamab) to the subject on Day 1 and/or Day 8 of the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the third phase.

In some examples, a target dose of the first bispecific antibody (e.g., an anti-FcRH5/anti-CD3 antibody, e.g., cevostamab) is administered to the subject during the pre-phase, the first phase, the second phase, and/or the third phase.

For example, during the pre-phase, a target dose of the first bispecific antibody (e.g., an anti-FcRH5/anti-CD3 antibody, e.g., cevostamab) may be administered to the subject on Day 9 of a dosing cycle of the pre-phase. In another example, a target dose of the first bispecific antibody (e.g., an anti-FcRH5/anti-CD3 antibody, e.g., cevostamab) may be administered to the subject on Day 10 of a dosing cycle of the pre-phase. In yet another example, a target dose of the first bispecific antibody (e.g., an anti-FcRH5/anti-CD3 antibody, e.g., cevostamab) may be administered to the subject on Day 11 of a dosing cycle of the pre-phase.

During the first phase, a target dose of the first bispecific antibody (e.g., an anti-FcRH5/anti-CD3 antibody, e.g., cevostamab) may be administered to the subject on Day 2, Day 3, or Day 4 of a dosing cycle of the first phase. In a particular example, a target dose of the first bispecific antibody (e.g., an anti-FcRH5/anti-CD3 antibody, e.g., cevostamab) may be administered to the subject on Day 2 of a dosing cycle of the first phase. In another example, a target dose of the first bispecific antibody (e.g., an anti-FcRH5/anti-CD3 antibody, e.g., cevostamab) may be administered to the subject on Day 3 of a dosing cycle of the first phase. In yet another example, a target dose of the first bispecific antibody (e.g., an anti-FcRH5/anti-CD3 antibody, e.g., cevostamab) may be administered to the subject on Day 4 of a dosing cycle of the first phase.

During the second phase, a target dose of the first bispecific antibody (e.g., an anti-FcRH5/anti-CD3 antibody, e.g., cevostamab) may be administered to the subject on Day 1 of a dosing cycle of the second phase.

In some embodiments, the method or treatment includes a third phase, wherein the third phase does not comprise the administration of the first bispecific antibody (e.g., an anti-FcRH5/anti-CD3 antibody, e.g., cevostamab) to the subject.

Any suitable target dose for the first bispecific antibody (e.g., an anti-FcRH5/anti-CD3 antibody, e.g., cevostamab) may be used, including any dosage described herein. In some examples, the target dose is between 20 mg to 600 mg (e.g., between 30 mg to 500 mg, 40 mg to 400 mg, 60 mg to 350 mg, 80 mg to 300 mg, 100 mg to 200 mg, or 140 mg to 180 mg, e.g., 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 mg). In some aspects, the target dose is between 40 mg to 80 mg (e.g., between 50 mg to 70 mg, e.g., 40 mg, 45 mg, 50 mg, 55 mg, 60 mg, 65 mg, 70 mg, 75 mg, or 80 mg). In some aspects, the target dose is between 80 mg to 300 mg. In some aspects the target dose is between 120 mg to 150 mg (e.g., between 130 mg to 140 mg, e.g., 120 mg, 125 mg, 130 mg, 131 mg, 132 mg, 133 mg, 134 mg, 135 mg, 136 mg, 137 mg, 138 mg, 139 mg, 140 mg, 145 mg, or 150 mg). In some aspects, the target dose is about 60 mg. In some aspects, the target dose is about 105 mg. In some aspects, the target dose is about 132 mg.

In some examples, during the pre-phase, a target dose of the second bispecific antibody (e.g., an anti-BCMA/anti-CD3 antibody, e.g., elranatamab) is administered to the subject on Day 8 (e.g., in a double step-up dosing regimen) of a dosing cycle of the pre-phase. In another example, a target dose of the second bispecific antibody (e.g., an anti-BCMA/anti-CD3 antibody, e.g., elranatamab) is administered on Day 15 of a dosing cycle of the pre-phase (e.g., in a double step-up dosing regimen). In yet another example, a target dose of the second bispecific antibody (e.g., an anti-BCMA/anti-CD3 antibody, e.g., elranatamab) is administered on Day 8 and Day 15 of a dosing cycle of the pre-phase (e.g., in a double step-up dosing regimen).

During the first phase, the method of treatment may comprise administering the second bispecific antibody (e.g., an anti-BCMA/anti-CD3 antibody, e.g., elranatamab) to the subject on Day 1 and/or Day 8 of a dosing cycle of the first phase. In a particular example, the second bispecific antibody (e.g., an anti-BCMA/anti-CD3 antibody, e.g., elranatamab) is administered to the subject on Day 1 of a dosing cycle of the first phase. In another example, the second bispecific antibody (e.g., an anti-BCMA/anti-CD3 antibody, e.g., elranatamab) is administered to the subject on Day 1 and Day 8 of a dosing cycle of the first phase.

During the second phase, the method of treatment may comprise administering the second bispecific antibody (e.g., an anti-BCMA/anti-CD3 antibody, e.g., elranatamab) to the subject on Day 1 and/or Day 8 of a dosing cycle of the second phase. In a particular example, the second bispecific antibody (e.g., an anti-BCMA/anti-CD3 antibody, e.g., elranatamab) is administered to the subject on Day 1 of a dosing cycle of the second phase. In another example, the second bispecific antibody (e.g., an anti-BCMA/anti-CD3 antibody, e.g., elranatamab) is administered to the subject on Day 8 of a dosing cycle of the second phase. In yet another example, the second bispecific antibody (e.g., an anti-BCMA/anti-CD3 antibody, e.g., elranatamab) is administered to the subject on Day 1 and Day 8 of a dosing cycle of the second phase.

During the third phase, the method of treatment may comprise administering the second bispecific antibody (e.g., an anti-BCMA/anti-CD3 antibody, e.g., elranatamab) to the subject on Day 1 and/or Day 8 of a dosing cycle of the third phase. In a particular example, the second bispecific antibody (e.g., an anti-BCMA/anti-CD3 antibody, e.g., elranatamab) is administered to the subject on Day 1 of a dosing cycle of the third phase. In another example, the second bispecific antibody (e.g., an anti-BCMA/anti-CD3 antibody, e.g., elranatamab) is administered to the subject on Day 1 and Day 8 of a dosing cycle of the third phase.

Any suitable step-up dosage of the second bispecific antibody (e.g., an anti-BCMA/anti-CD3 antibody, e.g., elranatamab) may be used (e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, or about 49 mg). In some examples, the second bispecific antibody (e.g., an anti-BCMA/anti-CD3 antibody, e.g., elranatamab) is administered to the subject at a step-up dosage of about 5 mg to about 50 mg (e.g., about 10 mg to about 40 mg, about 10 mg to about 15 mg, about 12 mg to about 22 mg, about 20 mg to about 40 mg, about 30 mg to about 35 mg, e.g., about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, or about 32 mg).

In one example, the second bispecific antibody (e.g., an anti-BCMA/anti-CD3 antibody, e.g., elranatamab) is administered to the subject at a first step-up dosage of about 4 mg to about 25 mg (e.g., about 4 mg to about 20 mg, about 10 mg to about 20 mg, about 11 mg to about 15 mg, e.g., about 12 mg). In a further example, the second bispecific antibody (e.g., an anti-BCMA/anti-CD3 antibody, e.g., elranatamab) is administered to the subject at a second step-up dosage of about 25 mg to about 45 mg (e.g., about 27 mg to about 37 mg, about 30 mg to about 34 mg, e.g., about 32 mg or about 44 mg). In yet a further example, the second bispecific antibody (e.g., an anti-BCMA/anti-CD3 antibody, e.g., elranatamab) is administered to the subject at a first step-up dose of about 12 mg and a second step-up dosage of about 32 mg. In yet a further example, the second bispecific antibody (e.g., an anti-BCMA/anti-CD3 antibody, e.g., elranatamab) is administered to the subject at a step-up dose of about 32 mg or of about 44 mg. After administration of the step-up dosage(s), a target dose of the second bispecific antibody (e.g., an anti-BCMA/anti-CD3 antibody, e.g., elranatamab) may be administered to the subject as described herein.

Any suitable target dosage of the second bispecific antibody (e.g., an anti-BCMA/anti-CD3 antibody, e.g., elranatamab) may be used (e.g., about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 120 mg, about 125 mg, about 130 mg, about 135 mg, about 140 mg, about 145 mg, about 150 mg, about 155 mg). In some examples, the second bispecific antibody (e.g., an anti-BCMA/anti-CD3 antibody, e.g., elranatamab) is administered to the subject at a target dosage of about 40 mg to about 160 mg (e.g., about 40 mg to about 120 mg, e.g. about 40 mg to about 80 mg, e.g. about 44 mg to about 80 mg, e.g. about 44 mg to about 76 mg, e.g. about 60 mg to about 80 mg, e.g., about 70 mg to about 80 mg, e.g., about 74 mg to about 78 mg, e.g., about 74 mg, about 75 mg, about 76 mg, about 77 mg, or about 78 mg). In some examples, the second bispecific antibody (e.g., an anti-BCMA/anti-CD3 antibody, e.g., elranatamab) is administered to the subject at a target dosage of about 44 mg, about 76 mg, about 116 mg or about 152 mg. In some examples, the second bispecific antibody (e.g., an anti-BCMA/anti-CD3 antibody, e.g. elranatamab) is administered to the subject at a target dosage of about 76 mg.

During any dosing cycle of a dosing regimen described herein, the second bispecific antibody (e.g., an anti-BCMA/anti-CD3 antibody, e.g., elranatamab) may be administered to the subject after (e.g., about 1 min, 5 min, 10 min, 15 min, 20 min, 25 min, 30 min, 35 min, 40 min, 45 min, 50 min, 55 min, 1 hour, 2 hours, 3 hours, 4 hours, 5 hours, 6 hours, 7 hours, 8 hours, 9 hours, 10 hours, 11 hours, or 12 hours after) the administration of the first bispecific antibody (e.g., an anti-FcRH5/anti-CD3 antibody, e.g., cevostamab) to the subject. During any dosing cycle of a dosing regimen described herein, the second bispecific antibody (e.g., an anti-BCMA/anti-CD3 antibody, e.g., elranatamab) may be administered to the subject before (e.g., about 1 min, 5 min, 10 min, 15 min, 20 min, 25 min, 30 min, 35 min, 40 min, 45 min, 50 min, 55 min, 1 hour, 2 hours, 3 hours, 4 hours, 5 hours, 6 hours, 7 hours, 8 hours, 9 hours, 10 hours, 11 hours, or 12 hours before) the administration of the first bispecific antibody (e.g., an anti-FcRH5/anti-CD3 antibody, e.g., cevostamab) to the subject.

The first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) may be administered by any suitable administration route. In some examples, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered to the subject intravenously. In other examples, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered to the subject subcutaneously. In other examples, the first bispecific antibody (e.g., an anti-FcRH5/anti-CD3 antibody, e.g., cevostamab) is administered intravenously and the second bispecific antibody (e.g., an anti-BCMA/anti-CD3 antibody, e.g., elranatamab) is administered to the subject subcutaneously.

In some examples, the method or treatment further comprises administering a corticosteroid to the subject. Any suitable corticosteroid may be used, e.g., dexamethasone or methylprednisolone.

In some examples, the method or treatment further comprises administering a corticosteroid to the subject during the C1 of the pre-phase 1 hour (±15 minutes) before administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). The corticosteroid may be administered on the same day as the first and/or second bispecific antibody or on a different day from the first and/or bispecific antibody (e.g., one or more days before or after administration of any bispecific antibody).

In some examples, the method of treatment further comprises administering a corticosteroid to the subject during the pre-phase.

The corticosteroid may be administered on any suitable day during a dosing cycle in the pre-phase.

In some examples, the method of treatment further comprises administering a corticosteroid to the subject during the first phase.

The corticosteroid may be administered on any suitable day during a dosing cycle in the first phase.

The corticosteroid may be administered on any one or more days selected from: Day 1, Day 2, Day 3, Day 4, and/or Day 8 of a dosing cycle of the first phase (e.g., during C1 of the first phase). In a particular example, the corticosteroid is administered to the subject on Day 1 of a dosing cycle of the first phase (e.g., before or after administration of the second bispecific antibody). In another example, the corticosteroid is administered to the subject on Day 2 of a dosing cycle of the first phase (e.g., before or after administration of the first bispecific antibody). In yet another example, the corticosteroid is administered to the subject on Day 3 of a dosing cycle of the first phase (e.g., before or after administration of the first bispecific antibody). In yet another example, the corticosteroid is administered to the subject on Day 4 of a dosing cycle of the first phase (e.g., before or after administration of the first bispecific antibody). In yet another example, the corticosteroid is administered to the subject on Day 8 of a dosing cycle of the first phase (e.g., before or after administration of the second bispecific antibody).

In some examples, the method of treatment further comprises administering a corticosteroid to the subject during the second phase.

The corticosteroid may be administered on any suitable day during a dosing cycle in the second phase.

The corticosteroid may be administered on Day 1 and/or Day 8 of a dosing cycle of the second phase (e.g., during C1 of the second phase). In a particular example, the corticosteroid is administered to the subject on Day 1 of a dosing cycle of the second phase (e.g., before or after administration of any bispecific antibody). In another example, the corticosteroid is administered to the subject on Day 8 of a dosing cycle of the second phase (e.g., before or after administration of any bispecific antibody).

In some examples, the method of treatment further comprises administering a corticosteroid to the subject during the third phase.

The corticosteroid may be administered on any suitable day during a dosing cycle in the third phase.

The corticosteroid may be administered on Day 1 and/or Day 8 of a dosing cycle of the third phase (e.g., during C1 of the third phase). In a particular example, the corticosteroid is administered to the subject on Day 1 of a dosing cycle of the third phase (e.g., before or after administration of any bispecific antibody). In another example, the corticosteroid is administered to the subject on Day 8 of a dosing cycle of the third phase (e.g., before or after administration of any bispecific antibody).

In some examples, the corticosteroid is administered to the subject if the subject experienced a cytokine release syndrome (CRS) with the prior dose. In some examples, the corticosteroid is administered to the subject in the pre-phase, first phase, second phase, and/or third phase if the subject experienced a CRS event with the prior dose.

The corticosteroid may be administered by any suitable administration route. In some examples, the corticosteroid is administered to the subject intravenously or orally. In some examples, the corticosteroid is administered to the subject intravenously.

In some examples, the corticosteroid is administered to the subject intravenously prior to the administration of any bispecific antibody. In some examples, the corticosteroid is administered to the subject intravenously after the administration of any bispecific antibody.

The corticosteroid may be administered in any suitable amount of time prior to the administration of the bispecific antibody, e.g., about 1 min, 5 min, 10 min, 15 min, 20 min, 25 min, 30 min, 35 min, 40 min, 45 min, 50 min, 55 min, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the administration of any bispecific antibody. In some examples, the corticosteroid is administered to the subject intravenously about 1 hour prior to the administration of any bispecific antibody.

The corticosteroid may be administered in any suitable amount of time after the administration of any bispecific antibody, e.g., about 1 min, 5 min, 10 min, 15 min, 20 min, 25 min, 30 min, 35 min, 40 min, 45 min, 50 min, 55 min, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 2 days, 3 days, 4 days, 5 days, 6 days, 7 days, or 8 days after the administration of any bispecific antibody. In some examples, the corticosteroid is administered to the subject intravenously about 24 hours after the administration of any bispecific antibody. In some examples, the corticosteroid is administered to the subject intravenously about 7 days after the administration of any bispecific antibody. In some examples, the corticosteroid is administered to the subject intravenously about 24 hours and, again, about 7 days after the administration of any bispecific antibody.

In some examples, the corticosteroid is dexamethasone or methylprednisolone.

In some examples, the corticosteroid is dexamethasone.

The dexamethasone may be administered at any suitable dosage, e.g., from 1 mg to 100 mg. In some examples, the dexamethasone is administered to the subject at a dosage of about 20 mg to about 40 mg. In some examples, the dexamethasone is administered to the subject at a dosage of about 20 mg.

The methylprednisolone may be administered at any suitable dose, e.g., from 1 mg 10 to 400 mg. In some examples, the methylprednisolone is administered to the subject at a dosage of about 80 mg.

Any two suitable anti-FcRH5/anti-CD3 and anti-BCMA/anti-CD3 bispecific antibodies may be used, e.g., any bispecific antibodies disclosed herein (e.g., in Section H below).

In some examples, the first bispecific antibody (e.g., an anti-FcRH5/anti-CD3 antibody) is cevostamab and the second bispecific antibody (e.g., an anti-BCMA/anti-CD3 antibody) is elranatamab.

In some examples, the first bispecific antibody (e.g., an anti-FcRH5/anti-CD3 antibody, e.g., cevostamab) and the second bispecific antibody (e.g., an anti-BCMA/anti-CD3 antibody, e.g., elranatamab) are administered to the subject concurrently with one or more additional therapeutic agents. Any suitable additional therapeutic agent(s) may be used, including any disclosed herein.

In some examples, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) are administered to the subject prior to the administration of one or more additional therapeutic agents.

In some examples, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered to the subject subsequent to the administration of one or more additional therapeutic agents.

In some examples, the one or more additional therapeutic agents comprise an effective amount of tocilizumab.

In some examples, the subject has a CRS event, and the method further comprises treating the symptoms of the CRS event while suspending treatment with the bispecific antibodies.

In some examples, the method or treatment further comprises administering to the subject an effective amount of tocilizumab to treat the CRS event.

In some examples, the CRS event does not resolve or worsens within 24 hours of treating the symptoms of the CRS event, the method further comprising administering to the subject one or more additional doses of tocilizumab to manage the CRS event.

In some examples, tocilizumab is administered to the subject by intravenous infusion.

In some examples: (a) the subject weighs ≥30 kg, and tocilizumab is administered to the subject at a dose of 8 mg/kg; or (b) the subject weighs <30 kg, and tocilizumab is administered to the subject at a dose of 12 mg/kg.

In some examples, tocilizumab is administered to the subject 2 hours before administration of any bispecific antibody.

In some examples, the one or more additional therapeutic agents comprise an effective amount of acetaminophen or paracetamol.

Any suitable dose of acetaminophen or paracetamol may be used. In some examples, acetaminophen or paracetamol is administered to the subject at a dose of between about 500 mg to about 1000 mg.

Acetaminophen or paracetamol may be administered by any suitable administration route, including any administration route disclosed herein. In some examples, acetaminophen or paracetamol is administered to the subject orally.

In some examples, the one or more additional therapeutic agents comprise an effective amount of diphenhydramine.

Any suitable dose of diphenhydramine may be used. In some examples, diphenhydramine is administered to the subject at a dose of between about 25 mg to about 50 mg.

Diphenhydramine may be administered by any suitable administration route, including any administration route disclosed herein. In some examples, diphenhydramine is administered orally to the subject.

In another example, provided herein is a method of treating a subject having an R/R MM, the method comprising administering to the subject a first bispecific antibody that specifically binds to FcRH5 and CD3 (e.g., cevostamab) and a second bispecific antibody that specifically binds to BCMA and CD3 (e.g., elranatamab) in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein the second bispecific antibody is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is a method of treating a subject having an R/R MM, the method comprising administering to the subject a first bispecific antibody that specifically binds to FcRH5 and CD3 (e.g., cevostamab) and a second bispecific antibody that specifically binds to BCMA and CD3 (e.g., elranatamab) in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein the second bispecific antibody is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is a method of treating a subject having an R/R MM, the method comprising administering to the subject a first bispecific antibody that specifically binds to FcRH5 and CD3 (e.g., cevostamab) and a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein the second bispecific antibody is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase.

In another example, provided herein is a method of treating a subject having an R/R MM, the method comprising administering to the subject a first bispecific antibody that specifically binds to FcRH5 and CD3 (e.g., cevostamab) and a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein the second bispecific antibody is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In some examples, the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6).

In some examples, the first bispecific antibody comprises an anti-FcRH5 arm comprising a first binding domain comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b). In some examples, the first binding domain comprises a VH domain comprising an amino acid sequence of SEQ ID NO: 7 and a VL domain comprising an amino acid sequence of SEQ ID NO: 8.

In some examples, the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14).

In some examples, the first bispecific antibody that specifically binds to FcRH5 and CD3 comprises an anti-CD3 arm comprising a second binding domain comprising (a) a VH domain comprising an amino acid sequence having at least 95% sequence identity to the amino acid sequence of SEQ ID NO: 15; (b) a VL domain comprising an amino acid sequence having at least 95% sequence identity to the amino acid sequence of SEQ ID NO: 16; or (c) a VH domain as in (a) and a VL domain as in (b). In some examples, the second binding domain comprises a VH domain comprising an amino acid sequence of SEQ ID NO: 15 and a VL domain comprising an amino acid sequence of SEQ ID NO: 16.

In some examples, the first bispecific antibody is cevostamab.

In some examples, the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47).

In some examples, the second bispecific antibody that specifically binds to BCMA and CD3 comprises an anti-BCMA arm comprising a first binding domain comprising (a) a VH domain comprising an amino acid sequence having at least 95% sequence identity to the amino acid sequence of SEQ ID NO: 48; (b) a VL domain comprising an amino acid sequence having at least 95% sequence identity to the amino acid sequence of SEQ ID NO: 49; or (c) a VH domain as in (a) and a VL domain as in (b). In some examples, the first binding domain comprises a VH domain comprising an amino acid sequence of SEQ ID NO: 48 and a VL domain comprising an amino acid sequence of SEQ ID NO: 49.

In some examples, the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In some examples, the second bispecific antibody that specifically binds to BCMA and CD3 comprises an anti-CD3 arm comprising a second binding domain comprising (a) a VH domain comprising an amino acid sequence having at least 95% sequence identity to the amino acid sequence of SEQ ID NO: 61; (b) a VL domain comprising an amino acid sequence having at least 95% sequence identity to the amino acid sequence of SEQ ID NO: 62; or (c) a VH domain as in (a) and a VL domain as in (b). In some examples, the second binding domain comprises a VH domain comprising an amino acid sequence of SEQ ID NO: 61 and a VL domain comprising an amino acid sequence of SEQ ID NO: 62.

In some examples, the second bispecific antibody is elranatamab.

In one aspect, provided herein is a method of treating a subject having a cancer (e.g., an MM), the method comprising administering to the subject (i) an effective amount of a first bispecific antibody that specifically binds to FcRH5 and CD3 and (ii) an effective amount of a second bispecific antibody that specifically binds to BCMA and CD3, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In one aspect, provided herein is a bispecific antibody that specifically binds to FcRH5 and CD3 for use in treatment of a subject having a cancer (e.g., an MM), the treatment comprising administering to the subject (i) an effective amount of a first bispecific antibody that specifically binds to FcRH5 and CD3 and (ii) an effective amount of a second bispecific antibody that specifically binds to BCMA and CD3, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In one aspect, provided herein is a bispecific antibody that specifically binds to BCMA and CD3 for use in treatment of a subject having a cancer (e.g., an MM), the treatment comprising administering to the subject (i) an effective amount of a first bispecific antibody that specifically binds to FcRH5 and CD3 and (ii) an effective amount of a second bispecific antibody that specifically binds to BCMA and CD3, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In one aspect, provided herein is a method of treating a subject having a cancer (e.g., an MM), the method comprising administering to the subject (i) an effective amount of a first bispecific antibody that specifically binds to FcRH5 and CD3 and (ii) an effective amount of a second bispecific antibody that specifically binds to BCMA and CD3, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In one aspect, provided herein is a bispecific antibody that specifically binds to FcRH5 and CD3 for use in treatment of a subject having a cancer (e.g., an MM), the treatment comprising administering to the subject (i) an effective amount of a first bispecific antibody that specifically binds to FcRH5 and CD3 and (ii) an effective amount of a second bispecific antibody that specifically binds to BCMA and CD3, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In one aspect, provided herein is a bispecific antibody that specifically binds to BCMA and CD3 for use in treatment of a subject having a cancer (e.g., an MM), the treatment comprising administering to the subject (i) an effective amount of a first bispecific antibody that specifically binds to FcRH5 and CD3 and (ii) an effective amount of a second bispecific antibody that specifically binds to BCMA and CD3, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, provided herein is a method of treating a human subject having an R/R MM, the method comprising administering to the subject an effective amount of cevostamab and an effective amount of elranatamab.

In another example, provided herein is a method of treating a human subject having an R/R MM, the method comprising administering to the subject cevostamab and elranatamab in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is a method of treating a human subject having an R/R MM, the method comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In another example, provided herein is a method of treating a human subject having an R/R MM, the method comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In another example, provided herein is a method of treating a human subject having an R/R MM, the method comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60). In some examples, the pre-phase comprises an approximately 15-day dosing cycle (C1), wherein: cevostamab is administered to the subject at a step-up dose of 3.6 mg on Day 9, 10, or 11 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase. In other examples, the pre-phase comprises an approximately 22-day dosing cycle (C1), wherein: cevostamab is administered to the subject at a step-up dose of 3.6 mg on Day 16, Day 17, or Day 18 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase.

In another example, provided herein is a method of treating a human subject having an R/R MM, the method comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60). In some examples, the pre-phase comprises an approximately 15-day dosing cycle (C1), wherein: cevostamab is administered to the subject at a step-up dose of 3.6 mg on Day 9, 10, or 11 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase. In other examples, the pre-phase comprises an approximately 22-day dosing cycle (C1), wherein: cevostamab is administered to the subject at a step-up dose of 3.6 mg on Day 16, Day 17, or Day 18 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase.

In another example, provided herein is a method of treating a human subject having an R/R MM, the method comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, provided herein is a method of treating a human subject having an R/R MM, the method comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, provided herein is a method of treating a human subject having an R/R MM, the method comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62. In some examples, the method further comprising a pre-phase prior to the first phase. In some examples, the pre-phase comprises an approximately 15-day dosing cycle (C1), wherein: cevostamab is administered to the subject at a step-up dose of 3.6 mg on Day 9, 10, or 11 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase. In other examples, the pre-phase comprises an approximately 22-day dosing cycle (C1), wherein: cevostamab is administered to the subject at a step-up dose of 3.6 mg on Day 16, Day 17, or Day 18 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase.

In another example, provided herein is a method of treating a human subject having an R/R MM, the method comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62. In some examples, the method further comprising a pre-phase prior to the first phase. In some examples, the pre-phase comprises an approximately 15-day dosing cycle (C1), wherein: cevostamab is administered to the subject at a step-up dose of 3.6 mg on Day 9, 10, or 11 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase. In other examples, the pre-phase comprises an approximately 22-day dosing cycle (C1), wherein: cevostamab is administered to the subject at a step-up dose of 3.6 mg on Day 16, Day 17, or Day 18 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase.

In another example, provided herein is a method of treating a human subject having an R/R MM, the method comprising administering to the subject cevostamab and elranatamab in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is a method of treating a human subject having an R/R MM, the method comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase.

In another example, provided herein is a method of treating a human subject having an R/R MM, the method comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is a bispecific antibody that specifically binds to FcRH5 and CD3 for use in treatment of a subject having an MM, the treatment comprising administration of a first bispecific antibody that specifically binds to FcRH5 and CD3 (e.g., cevostamab) and a second bispecific antibody that specifically binds to BCMA and CD3 (e.g., elranatamab) to the subject.

In another example, provided herein is a bispecific antibody that specifically binds to FcRH5 and CD3 (e.g., cevostamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In another example, provided herein is a bispecific antibody that specifically binds to FcRH5 and CD3 (e.g., cevostamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a 30 second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In another example, provided herein is a bispecific antibody that specifically binds to FcRH5 and CD3 (e.g., cevostamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60). In some examples, the treatment further comprises a pre-phase prior to the first phase. In some examples, the pre-phase comprises an approximately 15-day dosing cycle (C1), wherein: cevostamab is administered to the subject at a step-up dose of 3.6 mg on Day 9, 10, or 11 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase. In other examples, the pre-phase comprises an approximately 22-day dosing cycle (C1), wherein: cevostamab is administered to the subject at a step-up dose of 3.6 mg on Day 16, Day 17, or Day 18 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase.

In another example, provided herein is a bispecific antibody that specifically binds to FcRH5 and CD3 (e.g., cevostamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60). In some examples, the treatment further comprises a pre-phase prior to the first phase. In some examples, the pre-phase comprises an approximately 15-day dosing cycle (C1), wherein: cevostamab is administered to the subject at a step-up dose of 3.6 mg on Day 9, 10, or 11 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase. In other examples, the pre-phase comprises an approximately 22-day dosing cycle (C1), wherein: cevostamab is administered to the subject at a step-up dose of 3.6 mg on Day 16, Day 17, or Day 18 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase.

In another example, provided herein is a bispecific antibody that specifically binds to FcRH5 and CD3 (e.g., cevostamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a 15 second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, provided herein is a bispecific antibody that specifically binds to FcRH5 and CD3 (e.g., cevostamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, provided herein is a bispecific antibody that specifically binds to FcRH5 and CD3 (e.g., cevostamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, provided herein is a bispecific antibody that specifically binds to FcRH5 and CD3 (e.g., cevostamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, provided herein is cevostamab for use in treatment of a subject having an R/R MM, the treatment comprising administering to the subject cevostamab and elranatamab in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is cevostamab for use in treatment of a subject having an R/R MM, the treatment comprising administering to the subject cevostamab and elranatamab in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is cevostamab for use in treatment of a subject having an R/R MM, the treatment comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase.

In another example, provided herein is cevostamab for use in treatment of a subject having an R/R MM, the treatment comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is a bispecific antibody that specifically binds to BCMA and CD3 (e.g., elranatamab) for use in treatment of a subject having an MM, the treatment comprising administration of a first bispecific antibody that specifically binds to FcRH5 and CD3 (e.g., cevostamab) and a second bispecific antibody that specifically binds to BCMA and CD3 to the subject.

In another example, provided herein is a bispecific antibody that specifically binds to BCMA and CD3 (e.g., elranatamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In another example, provided herein is a bispecific antibody that specifically binds to BCMA and CD3 (e.g., elranatamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In another example, provided herein is a bispecific antibody that specifically binds to BCMA and CD3 (e.g., elranatamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase, wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In another example, provided herein is a bispecific antibody that specifically binds to BCMA and CD3 (e.g., elranatamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase wherein: the first bispecific antibody that specifically binds to FcRH5 and CD3 comprises an anti-FcRH5 arm comprising a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6); the first bispecific antibody that specifically binds to FcRH5 and CD3 comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14); the second bispecific antibody that specifically binds to BCMA and CD3 comprises an anti-BCMA arm comprising a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47); and the second bispecific antibody that specifically binds to BCMA and CD3 comprises an anti-CD3 arm comprising a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In another example, provided herein is a bispecific antibody that specifically binds to BCMA and CD3 (e.g., elranatamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, a bispecific antibody that specifically binds to BCMA and CD3 (e.g., elranatamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein the first bispecific antibody that specifically binds to FcRH5 and CD3 is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, a bispecific antibody that specifically binds to BCMA and CD3 (e.g., elranatamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase, wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, a bispecific antibody that specifically binds to BCMA and CD3 (e.g., elranatamab) for use in treatment of a human subject having an R/R MM, the treatment comprising administering to the subject (i) an effective amount of a first bispecific antibody that specifically binds to FcRH5 and CD3 (e.g., cevostamab) and (ii) an effective amount of a second bispecific antibody that specifically binds to BCMA and CD3 (e.g., elranatamab) in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein the first bispecific antibody that specifically binds to FcRH5 and CD3 is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; wherein the second bispecific antibody that specifically binds to BCMA and CD3 is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase wherein: the first bispecific antibody comprises an anti-FcRH5 arm comprising a first binding domain comprising a VH domain comprising an amino acid sequence of SEQ ID NO: 7 and a VL domain comprising an amino acid sequence of SEQ ID NO: 8; the first bispecific antibody that specifically binds to FcRH5 and CD3 comprises an anti-CD3 arm comprising a second binding domain comprising a VH domain comprising an amino acid sequence of SEQ ID NO: 15 and a VL domain comprising an amino acid sequence of SEQ ID NO: 16; the second bispecific antibody that specifically binds to BCMA and CD3 comprises an anti-BCMA arm comprising a first binding domain comprising a VH domain comprising an amino acid sequence of SEQ ID NO: 48 and a VL domain comprising an amino acid sequence of SEQ ID NO: 49; and the second bispecific antibody that specifically binds to BCMA and CD3 comprises an anti-CD3 arm comprising a second binding domain comprising a VH domain comprising an amino acid sequence of SEQ ID NO: 61 and a VL domain comprising an amino acid sequence of SEQ ID NO: 62.

In another example, provided herein is elranatamab for use in treatment of a human subject having an R/R MM, the treatment comprising administering to the subject cevostamab and elranatamab in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is elranatamab for use in treatment of a human subject having an R/R MM, the treatment comprising administering to the subject cevostamab and elranatamab in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is elranatamab for use in treatment of a human subject having an R/R MM, the treatment comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase.

In another example, provided herein is elranatamab for use in treatment of a human subject having an R/R MM, the treatment comprising administering to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C2, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

B. Combination Therapies with Additional Therapeutic Agents

In some instances, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) are administered to the subject with one or more additional therapeutic agents, including any additional therapeutic agents disclosed herein.

i. Corticosteroids

In some instances, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) are administered to the subject in combination with a corticosteroid. The corticosteroid may be administered orally to the subject. The corticosteroid may be administered by any suitable administration route, e.g., intravenously or subcutaneously to the subject. Any suitable corticosteroid may be used, e.g., dexamethasone, methylprednisolone, prednisone, prednisolone, betamethasone, hydrocortisone, and the like. In some aspects, the corticosteroid is methylprednisolone. methylprednisolone may be administered to the subject at a dose of about 80 mg. In other aspects, the corticosteroid is dexamethasone. The dexamethasone may be administered to the subject at a dose of about 20 mg. In some aspects, the corticosteroid (e.g., methylprednisolone or dexamethasone) is administered to the subject prior to the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), e.g., administered one hour prior to the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). In some aspects, the corticosteroid (e.g., methylprednisolone or dexamethasone) is administered to the subject about one day prior to the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). In some aspects, the corticosteroid (e.g., methylprednisolone or dexamethasone) is administered to the subject concurrently with the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). In some aspects, the corticosteroid (e.g., methylprednisolone or dexamethasone) is administered to the subject after (e.g., about 1 min, 5 min, 10 min, 15 min, 20 min, 25 min, 30 min, 35 min, 40 min, 45 min, 50 min, 55 min, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 2 days, 3 days, 4 days, 5 days, 6 days, 7 days, or 8 days) the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). In some examples, the corticosteroid (e.g., dexamethasone) is administered to the subject intravenously about 24 hours after the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). In some examples, the corticosteroid is administered to the subject intravenously about 7 days after the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). In some examples, the corticosteroid is administered to the subject intravenously about 24 hours and again about 7 days after the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab).

ii. Tocilizumab and Treatment of CRS

In one instance, the additional therapeutic agent is an effective amount of tocilizumab (ACTEMRA®). In some instances, the subject has a cytokine release syndrome (CRS) event (e.g., has a CRS event following treatment with the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab)), and the method further comprises treating the symptoms of the CRS event (e.g., treating the CRS event by administering to the subject an effective amount of tocilizumab) while suspending treatment with the first and second bispecific antibodies. In some aspects, tocilizumab is administered intravenously to the subject as a single dose of about 8 mg/kg. In some aspects, the CRS event does not resolve or worsens within 24 hours of treating the symptoms of the CRS event, and the method further comprising administering to the subject one or more additional doses of tocilizumab to manage the CRS event, e.g., administering one or more additional doses of tocilizumab intravenously to the subject at a dose of about 8 mg/kg.

In some aspects, treating the symptoms of the CRS event further comprises treatment with a high-dose vasopressor (e.g., norepinephrine, dopamine, phenylephrine, epinephrine, or vasopressin and norepinephrine), e.g., as described in Tables 2A and 2B.

In other instances, tocilizumab is administered as a premedication, e.g., is administered to the subject prior to the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). In some aspects, the tocilizumab is administered intravenously to the subject as a single dose of about 8 mg/kg.

iii. CRS Symptoms and Grading

CRS may be graded according to the Modified Cytokine Release Syndrome Grading System established by Lee et al., Blood, 124: 188-195, 2014 or Lee et al., Biol Blood Marrow Transplant, 25(4): 625-638, 2019, as described in Table 1A. In addition to diagnostic criteria, recommendations on management of CRS based on its severity, including early intervention with corticosteroids and/or anti-cytokine therapy, are provided and referenced in Tables 1A and 1B.

TABLE 1A
Cytokine release syndrome grading systems

| | Modified Cytokine Release Syndrome | |
|---|---|---|
| Grade | Grading System | ASTCT Consensus Grading System |
| Grade 1 | Symptoms are not life threatening and | Temperature ≥38° C. |
| | require symptomatic treatment only (e.g., | No hypotension |
| | fever, nausea, fatigue, headache, myalgia, | No hypoxia |
| | malaise) | |
| Grade 2 | Symptoms require and respond | Temperature ≥38° C.* with |
| | to moderate intervention | hypotension not requiring |
| | Oxygen requirement <40%; or | vasopressors and/or† hypoxia |
| | Hypotension responsive to fluids or | requiring low-flow nasal cannula‡ |
| | low dose ^{a} of one vasopressor; or | or blow-by |
| | Grade 2 organ toxicity | |
| Grade 3 | Symptoms require and respond | Temperature ≥38° C.* with |
| | to aggressive intervention | hypotension requiring a vasopressor |
| | Oxygen requirement ≥40%; or | with or without vasopressin and/or† |
| | Hypotension requiring high dose ^{b} | hypoxia requiring high-flow nasal |
| | or multiple vasopressors; or | cannula‡, facemask, nonrebreather |
| | Grade 3 organ toxicity or Grade 4 | mask, or Venturi mask |
| | transaminitis | |
| Grade 4 | Life-threatening symptoms | Temperature ≥38° C.* with |
| | Requirement for ventilation support | hypotension requiring multiple |
| | or Grade 4 organ toxicity (excluding | vasopressors (excluding vasopressin) |
| | transaminitis) | and/or† hypoxia requiring positive |
| | | pressure (e.g., CPAP, BiPAP, |
| | | intubation and mechanical ventilation) |
| Grade 5 | Death | Death |

Lee 2014 criteria: Lee et al., Blood, 124: 188-195, 2014.
ASTCT consensus grading: Lee et al., Biol Blood Marrow Transplant, 25(4): 625-638, 2019.
^{a} Low-dose vasopressor: single vasopressor at doses below that shown in Table 1B.
^{b} High-dose vasopressor: as defined in Table 1B.
*Fever is defined as temperature ≥38° C. not attributable to any other cause. In subjects who have CRS then receive antipyretic or anticytokine therapy such as tocilizumab or steroids, fever is no longer required to grade subsequent CRS severity. In this case, CRS grading is driven by hypotension and/or hypoxia.
†CRS grade is determined by the more severe event: hypotension or hypoxia not attributable to any other cause. For example, a subject with temperature of 39.5° C., hypotension requiring 1 vasopressor, and hypoxia requiring low-flow nasal cannula is classified as grade 3 CRS.
‡Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

TABLE 1B
High-dose vasopressors
High-Dose Vasopressors (duration ≥3 hours)

| Pressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine |
| | equivalent of ≥10 μg/min ^{a} |
| If on combination or vasopressors | Norepinephrine equivalent |
| (not vasopressin) | of ≥20 μg/min ^{a} |

min = minute; VASST = Vasopressin and Septic Shock Trial.
^{a} VASST vasopressor equivalent equation: norepinephrine equivalent dose = [norepinephrine (μg/min)] + [dopamine (μg/kg/min) ÷ 2] + [epinephrine (μg/min)] + [phenylephrine (μg/min) ÷ 10].

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS). Standard of care for severe or life-threatening CRS resulting from immune-based therapy has not been established; case reports and recommendations using anti-cytokine therapy such as tocilizumab have been published (Teachey et al., Blood, 121: 5154-5157, 2013; Lee et al., Blood, 124: 188-195, 2014; Maude et al., New Engl J Med, 371: 1507-1517, 2014).

As noted in Table TA, even moderate presentations of CRS in subjects with extensive comorbidities should be monitored closely, with consideration given to intensive care unit admission and tocilizumab administration.

iv. Tocilizumab Administered to Treat CRS

In some aspects, the subject experiences a CRS event during treatment with the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) and an effective amount of an IL-6R antagonist (e.g., an anti-IL-6R antibody, e.g., tocilizumab (ACTEMRA®/ROACTEMRA®)) is administered to manage the CRS event.

In some aspects, the subject has a CRS event (e.g., has a CRS event following treatment with the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), e.g., has a CRS event following a first dose or a subsequent dose of the first bispecific antibody and/or the second bispecific antibody), and the method further includes treating the symptoms of the CRS event while suspending treatment of the first bispecific antibody and/or the second bispecific antibody.

In some aspects, the subject experiences a CRS event, and the method further includes administering to the subject an effective amount of an interleukin-6 receptor (IL-6R) antagonist (e.g., an anti-IL-6R antibody, e.g., tocilizumab (ACTEMRA®/ROACTEMRA®)) to manage the CRS event while suspending treatment of the first bispecific antibody and/or the second bispecific antibody. In some aspects, the IL-6R antagonist (e.g., tocilizumab) is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

In some aspects, the CRS event does not resolve or worsens within 24 hours of treating the symptoms of the CRS event, and the method further includes administering to the subject one or more additional doses of the IL-6R antagonist (e.g., an anti-IL-6R antibody, e.g., tocilizumab) to manage the CRS event, e.g., administering one or more additional doses of tocilizumab intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the one or more additional doses of tocilizumab are administered intravenously to the subject as a single dose of about 8 mg/kg.

In some aspects, the method further includes administering to the subject an effective amount of a corticosteroid. The corticosteroid may be administered intravenously to the subject. In other examples, the corticosteroid may be administered subcutaneously to the subject. In some aspects, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

The subject may be administered a corticosteroid, such as methylprednisolone or dexamethasone, if the CRS event is not managed with administration of the IL-6R antagonist (e.g., tocilizumab) alone. In some aspects, treating the symptoms of the CRS event further includes treatment with a high-dose vasopressor (e.g., norepinephrine, dopamine, phenylephrine, epinephrine, or vasopressin and norepinephrine), e.g., as described in Tables TA and Table 1B. Tables 2A and 2B further provide details about tocilizumab treatment of severe or life-threatening CRS.

v. Management of CRS Events by Grade

Management of the CRS events may be tailored based on the grade of the CRS (Tables 1A and 2A) and the presence of comorbidities. Table 2A provides recommendations for the management of CRS syndromes by grade. Table 2B provides recommendations for the management of IRR syndromes by grade.

TABLE 2A
Recommendations for management of cytokine release syndrome (CRS)

| Event ^{a, b} | Action to Be Taken ^{b} |
|---|---|
| Grade 1 | Immediate actions: |
| Fever (≥38° C. or 100.4° F. not | If cevostamab infusion is still ongoing, interrupt |
| attributable to any other | infusion immediately. |
| cause) | Call to ensure tocilizumab is readily available if |
| | needed. |
| | Provide supportive care for constitutional |
| | symptoms. ^{c} |
| | Treat fever and neutropenia if present. |
| | Monitor fluid balance; administer IV fluids as |
| | clinically indicated. |
| | Consider hospitalization until symptoms |
| | completely resolve. |
| | In case of rapid decline in overall health or |
| | prolonged CRS |
| | (>24 hours) or in subjects with significant |
| | symptoms and/or comorbidities (per investigator |
| | discretion, e.g., impaired cardiovascular function, |
| | reduced pulmonary reserve), consider |
| | administration of IV corticosteroids and |
| | tocilizumab or another anti-cytokine agent based |
| | on characterization of CRS. |
| | Restarting infusion: |
| | If cevostamab infusion was interrupted, wait until |
| | 30 minutes after the event has resolved before |
| | restarting the infusion at 50% of the original |
| | infusion rate. |
| | If symptoms recur, discontinue infusion for this |
| | dose. |
| | Next dose: |
| | Pretreat with antihistamines, antipyretics, |
| | and/or analgesics. |
| | Pretreat with IV corticosteroids |
| | (dexamethasone 20 mg (preferred) or |
| | methylprednisolone 80 mg). |
| | Consider hospitalization for next dose. |
| | Consider extending infusion time (slower |
| | infusion rate) for subsequent cycles. |
| Grade 2 | Immediate actions: |
| Fever (≥38° C. or 100.4° F. not | Follow all Grade 1 recommendations. |
| attributable to any other cause) | Hold further cevostamab treatment until symptoms |
| Hypotension: responds to | completely resolved. |
| fluids, not requiring | Hospitalize subject until complete resolution of |
| vasopressors | signs andsymptoms. |
| Hypoxia: requiring low flow | Administer tocilizumab 8 mg/kg IV ^{d} |
| nasal cannula or blow-by | Consider treatment with IV corticosteroids (such as |
| | methylprednisolone 2 mg/kg/day or, if neurologic |
| | symptoms are |
| | present, dexamethasone 10 mg IV every 6 hours if |
| | needed). ^{b} |
| | Monitor cardiac and other organ function closely. |
| | Manage constitutional symptoms and organ |
| | toxicities as required. |
| | Provide hemodynamic support with IV fluid bolus |
| | 250-500 mL up to 2000 mL total (avoid fluid overload). |
| | Provide oxygen for hypoxia. |
| | Admit to ICU as appropriate. |
| | If no improvement within 8-12 hours after |
| | administration of tocilizumab, manage as a Grade 3 |
| | event: |
| | Initiate workup and assess for signs and |
| | symptoms of MAS/HLH. |
| | Restarting infusion: |
| | Wait until 30 minutes after the event has resolved |
| | before restarting the infusion at up to 25% of the |
| | original infusion rate. |
| | If symptoms recur, stop infusion immediately. |
| | Cevostamab should not be restarted. |
| | If hypotension or hypoxia recurs, manage as a |
| | Grade 3 event. |
| | Next cycle: |
| | May receive the next dose of cevostamab if |
| | symptoms resolve to Grade ≤1 for 3 consecutive |
| | days as follows: |
| | Consider hospitalization for at least 24 hours after |
| | the infusion. |
| | Administer cevostamab at 50% of the initial |
| | infusion rate of the previous cycle if the event |
| | occurred during or within 24 hours of the infusion. ^{e} |
| | Pretreat with antihistamines, antipyretics, and/or |
| | analgesics. |
| | Pretreat with IV corticosteroids (dexamethasone |
| | 20 mg (preferred) or methylprednisolone 80 mg). |
| | Subsequent cycles: |
| | If there is an occurrence of IRR or CRS Grade ≥3 |
| | in any of the subsequent cycles, permanently |
| | discontinue cevostamab regardless of recovery (see |
| | Grade 3 management guidelines). |
| | If there is an occurrence of a Grade ≤2 CRS in |
| | subsequent cycles, manage as indicated by severity |
| | (see Grade 1 or 2 management guidelines). |
| Grade 3 | Immediate actions: |
| Fever (≥38° C. or 100.4° F. not | Stop further infusion of cevostamab. |
| attributable to any other cause) | Provide supportive care and treat symptomatically |
| Hypotension: requires a | as clinically indicated. ^{c} |
| vasopressor with or without | Monitor fluid balance; administer IV fluids bolus |
| vasopressin | 250-500 mL up to 2000 mL total (avoid fluid |
| Hypoxia: requiring a high-flow | overload); provide vasopressor support for |
| nasal cannula, face mask, non- | hypotension with high and repeated doses if |
| rebreather mask, or Venturi | required. |
| mask | Hospitalize subject until complete resolution of |
| | signs and symptoms. |
| | Treat with IV corticosteroids (such as |
| | methylprednisolone 2 mg/kg/day or, if neurologic |
| | symptoms are present, |
| | dexamethasone 10 mg IV every 6 hours if needed). ^{b} |
| | Administer tocilizumab 8 mg/kg IV. ^{d} |
| | If there is no improvement after 8 hours, repeat |
| | tocilizumab |
| | If no improvement within 8-12 hours after |
| | administering second dose of tocilizumab, manage |
| | as a Grade 4 event. |
| | Initiate workup and assess for signs and symptoms |
| | of MAS/HLH. |
| | Monitoring of cardiopulmonary and organ function |
| | in ICU is recommended. |
| | Provide oxygen for hypoxia. |
| | Restarting infusion: |
| | Do not restart infusion of cevostamab. |
| | Next cycle: |
| | If the subject had a Grade ≥2 IRR or CRS in any |
| | previous not cycle, permanently discontinue |
| | cevostamab. |
| | Subjects who experience Grade 3 wheezing, |
| | bronchospasm, or generalized urticaria must be |
| | discontinued from cevostamab. |
| | If the subject recovers to Grade ≤1 for 3 |
| | consecutive days, cevostamab can be administered |
| | in next cycle as follows: |
| | Hospitalize subject for at least 24 hours after the |
| | mask infusion. |
| | Administer cevostamab at 50% of the initial |
| | infusion rate of the previous cycle if the event |
| | occurred during or within 24 hours of the infusion. ^{e} |
| | Pretreat with antihistamines, antipyretics, and/or |
| | analgesics |
| | Pretreat with IV corticosteroids (dexamethasone |
| | 20 mg (preferred) or methylprednisolone 80 mg). |
| | If the subject experienced Grade 3 CRS following |
| | the Cycle 1 Day 1 dose, the step-up dose must be |
| | repeated. |
| | Subsequent cycles: |
| | If a Grade ≥3 CRS recurs, permanently discontinue |
| | cevostamab. |
| | If there is an occurrence of a Grade ≤2 CRS in |
| | subsequent cycles, manage as indicated by severity |
| | (i.e., Grade 1 or 2 management guidelines). |
| Grade 4 | Stop cevostamab infusion. |
| Fever (≥38° C. or 100.4° F. not | Follow all Grade 3 management guidelines. |
| attributable to any other cause) | Subject requires ICU admission for |
| Hypotension: requires multiple | hemodynamic monitoring, and/or mechanical |
| vasopressors | ventilation, and/or IV fluids and vasopressors |
| Hypoxia: requiring positive | as needed. Administer tocilizumab 8 mg/kg IV |
| pressure (e.g., CPAP, BiPAP, | (see footnote). ^{d} |
| intubation, and mechanical | Treat with IV corticosteroids (such as |
| ventilation) | methylprednisolone 2 mg/kg/day or, if |
| | neurologic symptoms are present, |
| | dexamethasone 10 mg IV every 6 hours if |
| | needed) |
| | For subjects who are refractory to tocilizumab |
| | therapy, experimental therapies (e.g., |
| | siltuximab, anakinra, emapalumab, or LCK- |
| | inhibitors) ^{b} may be considered. |
| | Permanently discontinue study treatment. |

BiPAP = bilevel positive airway pressure; CPAP = continuous positive airway pressure; CRS = cytokine release syndrome; HLH = hemophagocytic lymphohistiocytosis; ICU
= intensive care unit; IV = intravenous; MAS = macrophage activation syndrome.
^{a} Refer to Table 1A for the complete description of grading of symptoms.
^{b} Guidance for CRS management based on Lee et al., Biol Blood Marrow Transplant, 25(4): 625-638, 2019 and Riegler et al. (2019).
^{c} Subjects should be treated with acetaminophen and an antihistamine (e.g., diphenhydramine) if they have not been administered in the previous 4 hours. For bronchospasm, urticaria, or dyspnea, treat per institutional practice. Treat fever and neutropenia as required; consider broad-spectrum antibiotics and/or G-CSF if indicated.
^{d} Tocilizumab should be administered at dose of 8 mg/kg IV (8 mg/kg for subjects ≥30 kg weight only; 12 mg/kg for subjects <30 kg weight; doses exceeding 800 mg per infusion are not recommended); repeat every 8 hours as necessary (up to a maximum of 4 doses).
^{e} If the subject does not experience CRS during the next infusion at the 50% reduced rate, the infusion rate can be increased to the initial rate in subsequent cycles. However, if this subject experiences another CRS event, the infusion rate should be reduced by 25%-50% depending on the severity of the event.

TABLE 2B
Recommendations for management of cevostamab
infusion related reactions (IRR)

| Event ^{a} | Action to Be Taken | |
|---|---|---|
| Grade 1-2 | Slow infusion to ≤50% or interrupt infusion. | |
| | Give supportive treatment. ^{b} | |
| | Upon symptom resolution, may resume infusion (if interrupted) | |
| | at 50% starting rate. The infusion must remain at the lower rate, | |
| | resulting in symptom resolution for the remainder of the | |
| | infusion.^{c} | |
| | Premedicate with acetaminophen/paracetamol and an | |
| | antihistamine such as diphenhydramine for all subsequent | |
| | infusions. | |
| | Notes: | |
| | 1. | For Grade 2 wheezing or urticaria, subject must be premedicated |
| | | prior to subsequent doses. |
| | 2. | If symptoms recur with the same or greater severity following the |
| | | slower or interrupted infusion of cevostamab, the infusion must be |
| | | stopped immediately. No further cevostamab should be |
| | | administered for the cycle. |

| | |
|---|---|
| Grade 3 | Discontinue cevostamab infusion. Do not re-initiate infusion for |
| | the current cycle. |
| | Give supportive treatment. ^{b} |
| | Admission to ICU is recommended to monitor cardiopulmonary |
| | and other organ functions. |
| | Provide oxygen for hypoxia and/or mechanical ventilation as |
| | needed. |
| | Subsequent cycles of cevostamab may be administered with |
| | premedication. |
| | Subjects who experience Grade 3 wheezing, bronchospasm, or |
| | generalized urticaria at first occurrence must be discontinued from |
| | study treatment. |
| | Notes: |
| | 1. If symptoms recur despite premedications with the same or |
| | greater |
| | severity at subsequent cycles, the infusion must be stopped |
| | immediately and subject permanently discontinued from study |
| | treatment. |
| Grade 4 | Discontinue infusion immediately. |
| | Give supportive treatment. ^{b} |
| | Admit to ICU to monitor cardiopulmonary and other organ |
| | functions. |
| | Provide oxygen for hypoxia and/or mechanical ventilation as |
| | needed. |
| | Permanently discontinue study treatment. |

ICU = intensive care unit; NCI CTCAE = National Cancer Institute Common Terminology Criteria for Adverse Events.
^{a} Refer to NCI CTCAE v5.0 for the grading of symptoms.
^{b} Supportive treatment: Subjects should be treated with acetaminophen/paracetamol and an antihistamine such as diphenhydramine if they have not been administered in
the last 4 hours. Intravenous fluids (e.g., normal saline) may be administered as clinically indicated. For bronchospasm, urticaria, or dyspnea, antihistamines, oxygen, corticosteroids (e.g., 100 mg IV prednisolone or equivalent), and/or bronchodilators may be administered per institutional practice. Provide fluids and vasopressor support for hypotension if required.
^{c} Subsequent infusions of cevostamab may be started at the original rate.

vi. Management of Grade 2 CRS Events

If the subject has a grade 2 CRS event (e.g., a grade 2 CRS event in the absence of comorbidities or in the presence of minimal comorbidities) following administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), the method may further include treating the symptoms of the grade 2 CRS event while suspending treatment of the first bispecific antibody and/or the second bispecific antibody. If the grade 2 CRS event then resolves to a grade ≤1 CRS event for at least three consecutive days, the method may further include resuming treatment with the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) without altering the dose. On the other hand, if the grade 2 CRS event does not resolve or worsens to a grade ≥3 CRS event within 24 hours of treating the symptoms of the grade 2 CRS event, the method may further involve administering to the subject an effective amount of an interleukin-6 receptor (IL-6R) antagonist (e.g., an anti-IL-6R antibody, e.g., tocilizumab (ACTEMRA®/ROACTEMRA®)) to manage the grade 2 or grade ≥3 CRS event. In some instances, tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

If the subject has a grade 2 CRS event in the presence of extensive comorbidities following administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), the method may further include administering to the subject a first dose of an IL-6R antagonist (e.g., an anti-IL-6R antibody, e.g., tocilizumab (ACTEMRA®/ROACTEMRA®)) to manage the grade 2 CRS event while suspending treatment of the first bispecific antibody and/or the second bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, if the grade 2 CRS event resolves to a grade ≤1 CRS event within two weeks, the method further includes resuming treatment with the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) at a reduced dose. In some instances, the reduced dose is 50% of the initial infusion rate of the previous cycle if the event occurred during or within 24 hours of the infusion. If, on the other hand, the grade 2 CRS event does not resolve or worsens to a grade ≥3 CRS event within 24 hours of treating the symptoms of the grade 2 CRS event, the method may further include administering to the subject one or more (e.g., one, two, three, four, or five or more) additional doses of an IL-6R antagonist (e.g., an anti-IL-6R antibody, e.g., tocilizumab) to manage the grade 2 or grade ≥3 CRS event. In some particular instances, the grade 2 CRS event does not resolve or worsens to a grade ≥3 CRS event within 24 hours of treating the symptoms of the grade 2 CRS event, and the method may further include administering to the subject one or more additional doses of tocilizumab to manage the grade 2 or grade ≥3 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or another anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

vii. Management of Grade 3 CRS Events

If the subject has a grade 3 CRS event following administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), the method may further include administering to the subject a first dose of an IL-6R antagonist (e.g., an anti-IL-6R antibody, e.g., tocilizumab (ACTEMRA®/ROACTEMRA®)) to manage the grade 3 CRS event while suspending treatment of the first bispecific antibody and/or the second bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, the subject recovers (e.g., is afebrile and off vasopressors) within 8 hours following treatment with the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), and the method further includes resuming treatment at a reduced dose. In some instances, the reduced dose is 50% of the initial infusion rate of the previous cycle if the event occurred during or within 24 hours of the infusion. In other instances, if the grade 3 CRS event does not resolve or worsens to a grade 4 CRS event within 24 hours of treating the symptoms of the grade 3 CRS event, the method may further include administering to the subject one or more (e.g., one, two, three, four, or five or more) additional doses of an IL-6R antagonist (e.g., an anti-IL-6R antibody, e.g., tocilizumab) to manage the grade 3 or grade 4 CRS event. In some particular instances, the grade 3 CRS event does not resolve or worsens to a grade 4 CRS event within 24 hours of treating the symptoms of the grade 3 CRS event, and the method further includes administering to the subject one or more additional doses of tocilizumab to manage the grade 3 or grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or another anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

viii. Management of Grade 4 CRS Events

If the subject has a grade 4 CRS event following administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), the method may further include administering to the subject a first dose of an IL-6R antagonist (e.g., an anti-IL-6R antibody, e.g., tocilizumab (ACTEMRA®/ROACTEMRA®)) to manage the grade 4 CRS event and permanently discontinuing treatment with the first bispecific antibody and/or the second bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. The grade 4 CRS event may, in some instances, resolve within 24 of treating the symptoms of the grade 4 CRS event. If the grade 4 CRS event does not resolve within 24 hours of treating the symptoms of the grade 4 CRS event, the method may further include administering to the subject one or more additional doses of an IL-6R antagonist (e.g., an anti-IL-6R antibody, e.g., tocilizumab (ACTEMRA®/ROACTEMRA®)) to manage the grade 4 CRS event. In some particular instances, the grade 4 CRS event does not resolve within 24 hours of treating the symptoms of the grade 4 CRS event, and the method further includes administering to the subject one or more (e.g., one, two, three, four, or five or more) additional doses of tocilizumab to manage the grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or another anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

ix. Acetaminophen or Paracetamol

In another instance, the additional therapeutic agent is an effective amount of acetaminophen or paracetamol. The acetaminophen or paracetamol may be administered orally to the subject, e.g., administered orally at a dose of between about 500 mg to about 1000 mg.

x. Diphenhydramine

In another instance, the additional therapeutic agent is an effective amount of diphenhydramine. The diphenhydramine may be administered orally to the subject, e.g., administered orally at a dose of between about 25 mg to about 50 mg.

### C. Premedication

i. Premedication with Corticosteroids, Acetaminophen or Paracetamol, and/or Diphenhydramine

Any of the methods or treatments disclosed herein may include pre-medication with a corticosteroid prior to administration of the first bispecific antibody and/or the second bispecific antibody to the subject.

Any of the methods or treatments disclosed herein may include pre-medication with acetaminophen or paracetamol prior to administration of the first bispecific antibody and/or the second bispecific antibody to the subject.

Any of the methods or treatments disclosed herein may include pre-medication with diphenhydramine prior to administration of the first bispecific antibody and/or the second bispecific antibody to the subject.

For example, any of the methods or treatments disclosed herein may include pre-medication with the following agents prior to administration of the first bispecific antibody and/or the second bispecific antibody to the subject: (i) a corticosteroid; (ii) acetaminophen or paracetamol; and/or (iii) diphenhydramine.

In some examples, the method or treatment comprises a pre-phase, and the corticosteroid is administered to the subject 1 hour (□ 15 minutes) prior to any administration of the first bispecific antibody and/or the second bispecific antibody during the pre-phase.

In some examples, the method or treatment comprises a first phase, and the corticosteroid is administered to the subject 1 hour (□ 15 minutes) prior to any administration of the first bispecific antibody and/or the second bispecific antibody during the first phase.

In some examples, the method or treatment comprises a second phase, wherein the subject has experienced CRS with a prior administration of the first bispecific antibody and/or the second bispecific antibody, and the corticosteroid is administered to the subject 1 hour (□ 15 minutes) prior to any administration of the first bispecific antibody and/or the second bispecific antibody during the second phase.

In some examples, the method or treatment comprises a third phase, wherein the subject has experienced CRS with a prior administration of the first bispecific antibody and/or the second bispecific antibody, and the corticosteroid is administered to the subject 1 hour (□ 15 minutes) prior to any administration of the second bispecific antibody during the third phase.

Any suitable corticosteroid may be used. In some examples, the corticosteroid is dexamethasone or methylprednisolone. In some examples, corticosteroid is dexamethasone. In some examples, the dexamethasone is administered to the subject at a dose of about 20 mg. In some examples, corticosteroid is methylprednisolone. In some examples, the methylprednisolone is administered to the subject at a dose of about 80 mg.

The corticosteroid may be administered by any suitable route. In some examples, the corticosteroid is administered to the subject intravenously.

In some examples, acetaminophen or paracetamol is administered to the subject at a dose of between 500 mg to 1000 mg. In some examples, acetaminophen or paracetamol is administered to the subject orally.

In some examples, diphenhydramine is administered to the subject at a dose of between 25 mg to 50 mg. In some examples, diphenhydramine is administered to the subject orally.

ii. Administration of Tocilizumab as a Premedication

In some aspects, an effective amount of an interleukin-6 receptor (IL-6R) antagonist (e.g., an anti-IL-6R antibody, e.g., tocilizumab (ACTEMRA®/ROACTEMRA®)) is administered as a premedication (prophylaxis), e.g., is administered to the subject prior to the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) (e.g., administered about 2 hours prior to the administration of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab)). Administration of tocilizumab as a premedication may reduce the frequency or severity of CRS. In some aspects, tocilizumab is administered as a premedication in Cycle 1, e.g., is administered prior to a first dose (C1D1; cycle 1, dose 1), a second dose (C1D2; cycle 1, dose, 2), and/or a third dose (C1D3; cycle 1, dose 3) of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). In some aspects, the tocilizumab is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. In some aspects, the tocilizumab is administered intravenously to the subject as a single dose of about 8 mg/kg for subjects weighing 30 kg or more (maximum 800 mg) and at a dose of about 12 mg/kg for subjects weighing less than 30 kg. Other anti-IL-6R antibodies that could be used in combination with tocilizumab include sarilumab, vobarilizumab (ALX-0061), SA-237, and variants thereof.

For example, in one aspect, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is co-administered with tocilizumab (ACTEMRA®/ROACTEMRA®), wherein the subject is first administered with tocilizumab (ACTEMRA®/ROACTEMRA®) and then separately administered with the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) (e.g., the subject is pre-treated with tocilizumab (ACTEMRA®/ROACTEMRA®)).

In some aspects, the incidence of CRS (e.g., Grade 1 CRS, Grade 2 CRS, and/or Grade 3+ CRS) is reduced in subjects who are treated with tocilizumab as a premedication relative to subjects who are not treated with tocilizumab as a premedication. In some aspects, less intervention to treat CRS (e.g., less need for additional tocilizumab, IV fluids, steroids, or O2) is required in subjects who are treated with tocilizumab as a premedication relative to subjects who are not treated with tocilizumab as a premedication. In some aspects, CRS symptoms have decreased severity (e.g., are limited to fevers and rigors) in subjects who are treated with tocilizumab as a premedication relative to subjects who are not treated with tocilizumab as a premedication.

### D. Methods of Administration

The methods and treatments may involve administering the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), and/or any additional therapeutic agent described herein by any suitable means, including parenteral, intrapulmonary, and intranasal, and, if desired for local treatment, intralesional administration. Parenteral infusions include intravenous, subcutaneous, intramuscular, intraarterial, and intraperitoneal administration routes. In some embodiments, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered by intravenous infusion. In other instances, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered subcutaneously.

In some instances, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) administered by intravenous injection exhibits a less toxic response (i.e., fewer unwanted effects) in a subject than the same first bispecific antibody and/or second bispecific antibody administered by subcutaneous injection, or vice versa.

In some aspects, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered intravenously over 4 hours (±15 minutes), e.g., the first dose of either antibody or both antibodies is administered over 4 hours±15 minutes.

In some aspects, the first dose and the second dose of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered intravenously with a median infusion time of less than four hours (e.g., less than three hours, less than two hours, or less than one hour) and further doses of the antibody are administered intravenously with a median infusion time of less than 120 minutes (e.g., less than 90 minutes, less than 60 minutes, or less than 30 minutes.

In some aspects, the first dose and the second dose of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered intravenously with a median infusion time of less than three hours and further doses of the antibody are administered intravenously with a median infusion time of less than 90 minutes.

In some aspects, the first dose and the second dose of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) is administered intravenously with a median infusion time of less than three hours and further doses of the antibody are administered intravenously with a median infusion time of less than 60 minutes. In some aspects, the subject is hospitalized (e.g., hospitalized for 72 hours, 48 hours, 24 hours, or less than 24 hours) during one or more administrations of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab). In some aspects, the subject is not hospitalized following the administration of any dose of the anti-FcRH5/anti-CD3 antibody.

In some examples, the first bispecific antibody (e.g., an anti-FcRH5/anti-CD3 antibody, e.g., cevostamab) is administered intravenously.

In some examples, the second bispecific antibody (e.g., an anti-BCMA/anti-CD3 antibody, e.g., elranatamab) is administered subcutaneously.

For all the methods described herein, the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), or any additional therapeutic agent(s) described herein, would be formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. The first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab), or any additional therapeutic agent(s) described herein, need not be, but is optionally formulated with, one or more agents currently used to prevent or treat the disorder in question. The effective amount of such other agents depends on the amount of the first bispecific antibody (e.g., an anti-FcRH5/anti-CD3 antibody, e.g., cevostamab) and/or the second bispecific antibody (e.g., an anti-BCMA/anti-CD3 antibody, e.g., elranatamab) present in the formulation, the type of disorder or treatment, and other factors discussed above. The first bispecific antibody (e.g., an anti-FcRH5/anti-CD3 antibody, e.g., cevostamab), the second bispecific antibody (e.g., an anti-BCMA/anti-CD3 antibody, e.g., elranatamab), and/or any additional therapeutic agent(s) described herein may be suitably administered to the subject over a series of treatments.

### E. Bispecific Antibodies

i. Anti-FcRH5/Anti-CD3 Bispecific Antibodies

The methods described herein include administering to a subject having a cancer (e.g., a hematologic cancer (e.g., a B cell proliferative disorder (e.g., an MM))) a bispecific antibody that specifically binds to FcRH5 and CD3 (i.e., a bispecific anti-FcRH5/anti-CD3 antibody). Any suitable bispecific antibody that specifically binds to FcRH5 and CD3 (i.e., a bispecific anti-FcRH5/anti-CD3 antibody) may be used.

In some instances, any of the methods described herein may include administering a bispecific antibody that includes an anti-FcRH5 arm having a first binding domain comprising at least one, two, three, four, five, or six hypervariable regions (HVRs) selected from (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6). In some instances, the bispecific anti-FcRH5/anti-CD3 antibody comprises at least one (e.g., 1, 2, 3, or 4) of the heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively.

In some instances, any of the methods described herein may include administering a bispecific antibody that includes an anti-FcRH5 arm having a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6). In some instances, the bispecific anti-FcRH5/anti-CD3 antibody comprises at least one (e.g., 1, 2, 3, or 4) of the heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively.

In some instances, the bispecific antibody comprises an anti-FcRH5 arm comprising a first binding domain comprising (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable (VL) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b). Accordingly, in some instances, the first binding domain comprises a VH domain comprising an amino acid sequence of SEQ ID NO: 7 and a VL domain comprising an amino acid sequence of SEQ ID NO: 8.

In some instances, any of the methods described herein may include administering a bispecific anti-FcRH5/anti-CD3 antibody that includes an anti-CD3 arm having a second binding domain comprising at least one, two, three, four, five, or six HVRs selected from (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14). In some instances, the anti-FcRH5/anti-CD3 bispecific antibody comprises at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively.

In some instances, any of the methods described herein may include administering a bispecific anti-FcRH5/anti-CD3 antibody that includes an anti-CD3 arm having a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14). In some instances, the anti-FcRH5/anti-CD3 bispecific antibody comprises at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively.

In some instances, the bispecific antibody comprises an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). Accordingly, in some instances, the second binding domain comprises a VH domain comprising an amino acid sequence of SEQ ID NO: 15 and a VL domain comprising an amino acid sequence of SEQ ID NO: 16.

In some instances, any of the methods described herein may include administering a bispecific antibody that includes (1) an anti-FcRH5 arm having a first binding domain comprising at least one, two, three, four, five, or six HVRs selected from (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6) and (2) an anti-CD3 arm having a second binding domain comprising at least one, two, three, four, five, or six HVRs selected from (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14).

In some instances, any of the methods described herein may include administering a bispecific antibody that includes (1) an anti-FcRH5 arm having a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6) and (2) an anti-CD3 arm having a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14).

In some instances, the anti-FcRH5/anti-CD3 bispecific antibody comprises (1) at least one (e.g., 1, 2, 3, or 4) of heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively, and (2) at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively. In some instances, the anti-FcRH5/anti-CD3 bispecific antibody comprises (1) all four of heavy chain framework regions FR-H1, FR-H2, FR-H3, and FR-H4 comprising the sequences of SEQ ID NOs: 17-20, respectively, and/or all four of the light chain framework regions FR-L1, FR-L2, FR-L3, and FR-L4 comprising the sequences of SEQ ID NOs: 21-24, respectively, and (2) all four of heavy chain framework regions FR-H1, FR-H2, FR-H3, and FR-H4 comprising the sequences of SEQ ID NOs: 25-28, respectively, and/or all four (e.g., 1, 2, 3, or 4) of the light chain framework regions FR-L1, FR-L2, FR-L3, and FR-L4 comprising the sequences of SEQ ID NOs: 29-32, respectively.

In some instances, the anti-FcRH5/anti-CD3 bispecific antibody comprises (1) an anti-FcRH5 arm comprising a first binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b), and (2) an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). In some instances, the anti-FcRH5/anti-CD3 bispecific antibody comprises (1) a first binding domain comprising a VH domain comprising an amino acid sequence of SEQ ID NO: 7 and a VL domain comprising an amino acid sequence of SEQ ID NO: 8 and (2) a second binding domain comprising a VH domain comprising an amino acid sequence of SEQ ID NO: 15 and a VL domain comprising an amino acid sequence of SEQ ID NO: 16.

In some instances, the anti-FcRH5/anti-CD3 bispecific antibody comprises an anti-FcRH5 arm comprising a heavy chain polypeptide (H1) and alight chain polypeptide (L1), wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 35 and/or (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 36.

In some instances, the anti-FcRH5/anti-CD3 bispecific antibody comprises an anti-FcRH5 arm comprising a heavy chain polypeptide (H1) and alight chain polypeptide (L1), wherein (a) H1 comprises the amino acid sequence of SEQ ID NO: 35 and/or (b) L1 comprises the amino acid sequence of SEQ ID NO: 36.

In some instances, the anti-FcRH5/anti-CD3 bispecific antibody comprises an anti-CD3 arm comprising a heavy chain polypeptide (H2) and a light chain polypeptide (L2), wherein (a) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 37 and/or (b) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 38.

In some instances, the anti-FcRH5/anti-CD3 bispecific antibody comprises an anti-CD3 arm comprising a heavy chain polypeptide (H2) and a light chain polypeptide (L2), wherein (a) H2 comprises the amino acid sequence of SEQ ID NO: 37; and (b) L2 comprises the amino acid sequence of SEQ ID NO: 38.

In some instances, the anti-FcRH5/anti-CD3 bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 35; (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 36; (c) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 37; and (d) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 38.

In some instances, the anti-FcRH5/anti-CD3 bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises the amino acid sequence of SEQ ID NO: 35; (b) L1 comprises the amino acid sequence of SEQ ID NO: 36; (c) H2 comprises the amino acid sequence of SEQ ID NO: 37; and (d) L2 comprises the amino acid sequence of SEQ ID NO: 38.

In some instances, the anti-FcRH5/anti-CD3 bispecific antibody is cevostamab.

ii. Anti-BCMA/Anti-CD3 Bispecific Antibodies

The methods and dosing regimens described herein include administering to a subject having a cancer (e.g., a hematologic cancer (e.g., a B cell proliferative disorder (e.g., an MM))) a bispecific antibody that specifically binds to BCMA and CD3 (i.e., a bispecific anti-BCMA/anti-CD3 antibody). Any suitable bispecific antibody that specifically binds to BCMA and CD3 (i.e., a bispecific anti-BCMA/anti-CD3 antibody) may be used. As used herein, an anti-BCMA/anti-CD3 bispecific antibody can be any molecule that can simultaneously bind to both BCMA (e.g., on B cells) and CD3 (e.g., on T cells). Anti-BCMA/anti-CD3 bispecific antibodies are also referred to herein as “BCMA x CD3” or “BCMA” bispecific antibodies.

B-cell maturation antigen (BCMA, also known as TNFRSF17 and CD269) is a candidate for bispecific antibody-based immunotherapy. BCMA expression is upregulated during B-cell maturation into plasma blasts and plasma cells, but it is not expressed on naïve B cells, hematopoietic stem cells or normal tissues such as the heart, lung, kidney, or tonsil. In multiple myeloma, BCMA expression was identified at each disease stage, and on patients with differing cytogenetic risks. Furthermore, BCMA expression was not influenced by treatment with autologous stem cell transplant (ASCT) or chemotherapy. In vivo, bispecific antibodies against BCMA have been shown to induce T-cell activation, reduce tumor burden and prolong survival.

Examples of anti-BCMA/anti-CD3 bispecific antibodies that may be useful in the combination therapies of the present disclosure include, but are not limited to, AMG 420 (BCMAxCD3 bispecific T-cell engager, BiTE®, Amgen), AMG 701 (BCMAxCD3 BiTE®, Amgen), CC-93269 (BCMAxCD3 bispecific antibody, Celgene), teclistamab (JNJ-64007957—Jansen), elranatamab (BCMAxCD3 bispecific antibody, Pfizer Inc.), TNB-383B (TeneoBio/AbbVie), linvoseltamab (REGN5458-BCMAxCD3 bispecific antibody, Regeneron), alnuctamab (CC-93269-BMS), AFM26 (BCMAxCD16 tetravalent bispecific antibody, Afimed GmbH), HPN217 (BCMAxALBxCD3 trispecific, Harpoon Therapeutics).

In some aspects, the anti-BCMA/anti-CD3 bispecific antibody comprises a first antigen binding site and a second antigen binding site, wherein the first antigen binding site specifically binds to BCMA, and wherein the second antigen binding site specifically binds to CD3.

In some aspects, an anti-BCMA/anti-CD3 bispecific antibody may have any of the features or characteristics of any of the BCMA bispecific antibodies provided in WO2016/166629, which is hereby incorporated by reference for all purposes.

In some aspects, the anti-BCMA/anti-CD3 bispecific antibody comprises an anti-BCMA arm that comprises a first binding domain that specifically binds to BCMA. Information about BCMA is provided, for example, via UniProtKB ID #Q02223. In some aspects, the first binding domain comprises three HVRs of a heavy chain variable region (VH) comprising the amino acid sequence shown in SEQ ID NO: 48, and/or three HVRs of a light chain variable region (VL) comprising the amino acid sequence shown in SEQ ID NO: 49. In some aspects, the VH comprises an HVR-H1 comprising the sequence shown in one or more of SEQ ID NO: 39, SEQ ID NO: 40, and SEQ ID NO: 41, an HVR-H2 comprising the sequence shown in one or more of SEQ ID NO: 42 and SEQ ID NO: 43, and an HVR-H3 comprising the sequence shown in SEQ ID NO: 44, and/or the VL comprises a HVR-L1 comprising the sequence shown in SEQ ID NO: 45, a HVR-L2 comprising the sequence shown in SEQ ID NO: 46, and an HVR-L3 comprising the sequence shown in SEQ ID NO: 47. In some aspects, the VH comprises the sequence shown in SEQ ID NO: 48, and/or the VL comprises the sequence shown in SEQ ID NO: 49. In some aspects, the bispecific antibody comprises a heavy chain and a light chain, wherein the heavy chain comprises the amino acid sequence shown in SEQ ID NO: 50, and/or the second light chain comprises the amino acid sequence shown in SEQ ID NO: 51.

In some aspects, the anti-BCMA/anti-CD3 bispecific antibody comprises an anti-CD3 arm that comprises a second binding domain that specifically binds to CD3. Information about CD3 is provided, for example, via UniProtKB #P07766. In some aspects, the first antigen binding site comprises three HVRs of a VH comprising the amino acid sequence shown in SEQ ID NO: 61, and/or three HVRs of a VL comprising the amino acid sequence shown in SEQ ID NO: 62. In some aspects, the VH comprises an HVR-H1 comprising the sequence shown in one or more of SEQ ID NO: 52, SEQ ID NO: 53, and SEQ ID NO: 54, an HVR-H2 comprising the sequence shown in one or more of SEQ ID NO: 55 and SEQ ID NO: 56, an HVR-H3 comprising the sequence shown in SEQ ID NO: SEQ ID NO: 57, and/or the VL comprises an HVR-L1 comprising the sequence shown in SEQ ID NO: 58, an HVR-L2 comprising the sequence shown in SEQ ID NO: 59, and an HVR-L3 comprising the sequence shown in SEQ ID NO: 60. In some aspects, the VH comprises the sequence shown in SEQ ID NO: 61, and/or the VL comprises the sequence shown in SEQ ID NO: 62. In some aspects, the bispecific antibody comprises a heavy chain and a light chain, wherein the heavy chain comprises the amino acid sequence shown in SEQ ID NO: 63, and/or the light chain comprises the amino acid sequence shown in SEQ ID NO: 64.

In some instances, any of the methods described herein may include administering a bispecific antibody that includes an anti-BCMA arm having a first binding domain comprising at least one, two, three, four, five, or six hypervariable regions (HVRs) selected from (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47).

In some instances, any of the methods described herein may include administering a bispecific antibody that includes an anti-BCMA arm having a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47).

In some instances, the bispecific antibody comprises an anti-BCMA arm comprising a first binding domain comprising (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 48; (b) a light chain variable (VL) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 49; or (c) a VH domain as in (a) and a VL domain as in (b). Accordingly, in some instances, the first binding domain comprises a VH domain comprising an amino acid sequence of SEQ ID NO: 48 and a VL domain comprising an amino acid sequence of SEQ ID NO: 49.

In some instances, any of the methods described herein may include administering a bispecific anti-BCMA/anti-CD3 antibody that includes an anti-CD3 arm having a second binding domain comprising at least one, two, three, four, five, or six HVRs selected from (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In some instances, any of the methods described herein may include administering a bispecific anti-BCMA/anti-CD3 antibody that includes an anti-CD3 arm having a second binding domain comprising the following six HVRs: (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In some instances, the bispecific antibody comprises an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 61; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 62; or (c) a VH domain as in (a) and a VL domain as in (b). Accordingly, in some instances, the second binding domain comprises a VH domain comprising an amino acid sequence of SEQ ID NO: 61 and a VL domain comprising an amino acid sequence of SEQ ID NO: 62.

In some instances, any of the methods described herein may include administering a bispecific antibody that includes (1) an anti-BCMA arm having a first binding domain comprising at least one, two, three, four, five, or six HVRs selected from (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47) and (2) an anti-CD3 arm having a second binding domain comprising at least one, two, three, four, five, or six HVRs selected from (a) an HVR-H1 comprising the amino acid sequence of GFTFSDY (SEQ ID NO: 53), DYYMT (SEQ ID NO: 52), or GFTFSDYYMT (SEQ ID NO: 54); (b) an HVR-H2 comprising the amino acid sequence of RNRARGYT (SEQ ID NO: 56) or FIRNRARGYTSDHNPSVKG (SEQ ID NO: 55); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In some instances, any of the methods described herein may include administering a bispecific antibody that includes (1) an anti-BCMA arm having a first binding domain comprising the following six HVRs: (a) an HVR-H1 comprising the amino acid sequence of GFTFSSY (SEQ ID NO: 40), SYPMS (SEQ ID NO: 39), or GFTFSSYPMS (SEQ ID NO: 41); (b) an HVR-H2 comprising the amino acid sequence of GGSGGS (SEQ ID NO: 43) or AIGGSGGSLPYADIVKG (SEQ ID NO: 42); (c) an HVR-H3 comprising the amino acid sequence of YWPMDI (SEQ ID NO: 44); (d) an HVR-L1 comprising the amino acid sequence of RASQSVSSSYLA (SEQ ID NO: 45); (e) an HVR-L2 comprising the amino acid sequence of DASIRAT (SEQ ID NO: 46); and (f) an HVR-L3 comprising the amino acid sequence of QQYQSWPLT (SEQ ID NO: 47) and (2) an anti-CD3 arm having a second binding domain comprising the following six HVRs: (a) an HVR-H1 comprising the amino acid sequence of DYYMT (SEQ ID NO: 52), GFTFSDY (SEQ ID NO: 53), or GFTFSDYYMT (SEQ ID NO: 54); (b) an HVR-H2 comprising the amino acid sequence of FIRNRARGYTSDHNPSVKG (SEQ ID NO: 55) or RNRARGYT (SEQ ID NO: 56); (c) an HVR-H3 comprising the amino acid sequence of DRPSYYVLDY (SEQ ID NO: 57); (d) an HVR-L1 comprising the amino acid sequence of KSSQSLFNVRSRKNYLA (SEQ ID NO: 58); (e) an HVR-L2 comprising the amino acid sequence of WASTRES (SEQ ID NO: 59); and (f) an HVR-L3 comprising the amino acid sequence of KQSYDLFT (SEQ ID NO: 60).

In some instances, the anti-BCMA/anti-CD3 bispecific antibody comprises (1) an anti-BCMA arm comprising a first binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 48; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 49; or (c) a VH domain as in (a) and a VL domain as in (b), and (2) an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 61; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 62; or (c) a VH domain as in (a) and a VL domain as in (b). In some instances, the anti-BCMA/anti-CD3 bispecific antibody comprises (1) a first binding domain comprising a VH domain comprising an amino acid sequence of SEQ ID NO: 48 and a VL domain comprising an amino acid sequence of SEQ ID NO: 49 and (2) a second binding domain comprising a VH domain comprising an amino acid sequence of SEQ ID NO: 61 and a VL domain comprising an amino acid sequence of SEQ ID NO: 62.

In some instances, the anti-BCMA/anti-CD3 bispecific antibody comprises an anti-BCMA arm comprising a heavy chain polypeptide (H1) and a light chain polypeptide (L1), wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 50 and/or (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 51.

In some instances, the anti-BCMA/anti-CD3 bispecific antibody comprises an anti-BCMA arm comprising a heavy chain polypeptide (H1) and a light chain polypeptide (L1), wherein (a) H1 comprises the amino acid sequence of SEQ ID NO: 50 and/or (b) L1 comprises the amino acid sequence of SEQ ID NO: 51.

In some instances, the anti-BCMA/anti-CD3 bispecific antibody comprises an anti-CD3 arm comprising a heavy chain polypeptide (H2) and a light chain polypeptide (L2), wherein (a) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 63 and/or (b) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 64.

In some instances, the anti-BCMA/anti-CD3 bispecific antibody comprises an anti-CD3 arm comprising a heavy chain polypeptide (H2) and a light chain polypeptide (L2), wherein (a) H2 comprises the amino acid sequence of SEQ ID NO: 63; and (b) L2 comprises the amino acid sequence of SEQ ID NO: 64.

In some instances, the anti-BCMA/anti-CD3 bispecific antibody comprises an anti-BCMA arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 50; (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 51; (c) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 63; and (d) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 64.

In some instances, the anti-BCMA/anti-CD3 bispecific antibody comprises an anti-BCMA arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises the amino acid sequence of SEQ ID NO: 50; (b) L1 comprises the amino acid sequence of SEQ ID NO: 51; (c) H2 comprises the amino acid sequence of SEQ ID NO: 63; and (d) L2 comprises the amino acid sequence of SEQ ID NO: 64.

In some embodiments, the antibody comprises a modified constant region, such as a constant region that is immunologically inert, that is, having a reduced potential for provoking an immune response. In some embodiments, the constant region is modified as described in Eur. J. Immunol., 29:2613-2624, 1999; PCT Application No. PCT/GB99/01441; and/or UK Patent Application No. 98099518. The Fc can be human IgG1, human IgG2, human IgG3, or human IgG4. The Fc can be human IgG2 containing the mutation A330P331 to S330S331 (IgG2Δa), in which the amino acid residues are numbered with reference to the wild type IgG2 sequence. Eur. J. Immunol., 29:2613-2624, 1999. In some embodiments, the antibody comprises a constant region of IgG4 comprising the following mutations (Armour et al., Molecular Immunology 40 585-593, 2003): E233F234L235 to P233V234A235 (IgG4Δc), in which the numbering is with reference to wild type IgG4. In yet another embodiment, the Fc is human IgG4 E233F234L235 to P233V234A235 with deletion G236 (IgG4Δb). In another embodiment, the Fc is any human IgG4 Fc (IgG4, IgG4Δb or IgG4Δc) containing hinge stabilizing mutation S228 to P228 (Aalberse et al., Immunology 105, 9-19, 2002). In another embodiment, the Fc can be aglycosylated Fc.

In some embodiments, the constant region is aglycosylated by mutating the oligosaccharide attachment residue (such as Asn297) and/or flanking residues that are part of the glycosylation recognition sequence in the constant region. In some embodiments, the constant region is aglycosylated for N-linked glycosylation enzymatically. The constant region may be aglycosylated for N-linked glycosylation enzymatically or by expression in a glycosylation deficient host cell.

In some embodiments, the constant region has a modified constant region that removes or reduces Fc gamma receptor binding. For example, the Fc can be human IgG2 containing the mutation D265A.

In some aspects, the anti-BCMA/anti-CD3 bispecific antibody is elranatamab. Elranatamab is a heterodimeric full-length bispecific antibody comprised of one B-cell maturation antigen (BCMA) binding arm and one cluster of differentiation (CD3) binding arm paired through hinge mutation technology. It utilizes a modified human IgG2Aa fragment crystallizable (Fc) region. Elranatamab is described, for example, in U.S. Pat. No. 9,969,809, which is hereby incorporated by reference in its entirety for all purposes. The sequences of elranatamab are shown in Table 3.

TABLE 3
Elranatamab Sequences

| SEQ ID NO | Description | Sequence |
|---|---|---|
| 40 | Anti-BCMA | GFTFSSY |
| | HCDR1 | |
| | Chothia | |
| 39 | Anti-BCMA | SYPMS |
| | HCDR1 | |
| | Kabat | |
| 41 | Anti-BCMA | GFTFSSYPMS |
| | HCDR1 | |
| | Extended | |
| 43 | Anti-BCMA | GGSGGS |
| | HCDR2 | |
| | Chothia | |
| 42 | Anti-BCMA | AIGGSGGSLPYADIVKG |
| | HCDR2 | |
| | Extended | |
| 44 | Anti-BCMA | YWPMDI |
| | HCDR3 | |
| 45 | Anti-BCMA | RASQSVSSSYLA |
| | LCDR1 | |
| 46 | Anti-BCMA | DASIRAT |
| | LCDR2 | |
| 47 | Anti-BCMA | QQYQSWPLT |
| | LCDR3 | |
| 48 | Anti-BCMA VH | EVQLLESGGG LVQPGGSLRL SCAASGFTFS SYPMSWVRQA |
| | | PGKGLEWVSA IGGSGGSLPY ADIVKGRFTI SRDNSKNTLY |
| | | LQMNSLRAED TAVYYCARYW PMDIWGQGTL VTVSS |
| 49 | Anti-BCMA VL | EIVLTQSPGT LSLSPGERAT LSCRASQSVS SSYLAWYQQK |
| | | PGQAPRLLMY DASIRATGIP DRFSGSGSGT DFTLTISRLE |
| | | PEDFAVYYCQ QYQSWPLTFG QGTKVEIK |
| 53 | Anti-CD3 | GFTFSDY |
| | HCDR1 Chothia | |
| 52 | Anti-CD3 | DYYMT |
| | HCDR1 | |
| | Kabat | |
| 54 | Anti-CD3 | GFTFSDYYMT |
| | HCDR1 | |
| | Extended | |
| 56 | Anti-CD3 | RNRARGYT |
| | HCDR2 | |
| | Chothia | |
| 55 | Anti-CD3 | FIRNRARGYTSDHNPSVKG |
| | HCDR2 | |
| | Extended | |
| 57 | Anti-CD3 | DRPSYYVLDY |
| | HCDR3 | |
| 58 | Anti-CD3 | KSSQSLFNVRSRKNYLA |
| | LCDR1 | |
| | Extended | |
| 59 | Anti-CD3 | WASTRES |
| | LCDR2 | |
| | Extended | |
| 60 | Anti-CD3 | KQSYDLFT |
| | LCDR3 | |
| 61 | Anti-CD3 VH | EVQLVESGGG LVQPGGSLRL SCAASGFTFS DYYMTWVRQA |
| | | PGKGLEWVAF IRNRARGYTS DHNPSVKGRF TISRDNAKNS |
| | | LYLQMNSLRA EDTAVYYCAR DRPSYYVLDY WGQGTTVTVS |
| | | S |
| 62 | Anti-CD3 VL | DIVMTQSPDS LAVSLGERAT INCKSSQSLF |
| | | NVRSRKNYLA WYQQKPGQPP KLLISWASTR |
| | | ESGVPDRFSG SGSGTDFTLT ISSLQAEDVA |
| | | VYYCKQSYDL FTFGSGTKLE IK |
| 50 | Anti-BCMA | EVQLLESGGG LVQPGGSLRL SCAASGFTFS |
| | heavy chain | SYPMSWVRQA PGKGLEWVSA IGGSGGSLPY |
| | | ADIVKGRFTI SRDNSKNTLY LQMNSLRAED |
| | | TAVYYCARYW PMDIWGQGTL VTVSSASTKG |
| | | PSVFPLAPCS RSTSESTAAL GCLVKDYFPE |
| | | PVTVSWNSGA LTSGVHTFPA VLQSSGLYSL |
| | | SSVVTVPSSN FGTQTYTCNV DHKPSNTKVD |
| | | KTVERKCEVE CPECPAPPVA GPSVFLFPPK |
| | | PKDTLMISRT PEVTCVVVAV SHEDPEVQFN |
| | | WYVDGVEVHN AKTKPREEQF NSTFRVVSVL |
| | | TVVHQDWLNG KEYKCKVSNK GLPSSIEKTI |
| | | SKTKGQPREP QVYTLPPSRE EMTKNQVSLT |
| | | CEVKGFYPSD IAVEWESNGQ PENNYKTTPP |
| | | MLDSDGSFFL YSKLTVDKSR WQQGNVFSCS |
| | | VMHEALHNHY TQKSLSLSPG K |
| 51 | Anti-BCMA | EIVLTQSPGT LSLSPGERAT LSCRASQSVS |
| | light chain | SSYLAWYQQK PGQAPRLLMY DASIRATGIP |
| | | DRFSGSGSGT DFTLTISRLE PEDFAVYYCQ |
| | | QYQSWPLTFG QGTKVEIKRT VAAPSVFIFP |
| | | PSDEQLKSGT ASVVCLLNNF YPREAKVQWK |
| | | VDNALQSGNS QESVTEQDSK DSTYSLSSTL |
| | | TLSKADYEKH KVYACEVTHQ GLSSPVTKSF NRGEC |
| 63 | Anti-CD3 | EVQLVESGGG LVQPGGSLRL SCAASGFTFS |
| | heavy chain | DYYMTWVRQA PGKGLEWVAF IRNRARGYTS |
| | | DHNPSVKGRF TISRDNAKNS LYLQMNSLRA |
| | | EDTAVYYCAR DRPSYYVLDY WGQGTTVTVS |
| | | SASTKGPSVF PLAPCSRSTS ESTAALGCLV |
| | | KDYFPEPVTV SWNSGALTSG VHTFPAVLQS |
| | | SGLYSLSSVV TVPSSNFGTQ TYTCNVDHKP |
| | | SNTKVDKTVE RKCRVRCPRC PAPPVAGPSV |
| | | FLFPPKPKDT LMISRTPEVT CVVVAVSHED |
| | | PEVQFNWYVD GVEVHNAKTK PREEQFNSTF |
| | | RVVSVLTVVH QDWLNGKEYK CKVSNKGLPS |
| | | SIEKTISKTK GQPREPQVYT LPPSREEMTK |
| | | NQVSLTCLVK GFYPSDIAVE WESNGQPENN |
| | | YKTTPPMLDS DGSFFLYSRL TVDKSRWQQG |
| | | NVFSCSVMHE ALHNHYTQKS LSLSPGK |
| 64 | Anti-CD3 light | DIVMTQSPDS LAVSLGERAT INCKSSQSLF |
| | chain | NVRSRKNYLA WYQQKPGQPP KLLISWASTR |
| | | ESGVPDRFSG SGSGTDFTLT ISSLQAEDVA |
| | | VYYCKQSYDL FTFGSGTKLE IKRTVAAPSV |
| | | FIFPPSDEQL KSGTASVVCL LNNFYPREAK |
| | | VQWKVDNALQ SGNSQESVTE QDSKDSTYSL |
| | | SSTLTLSKAD YEKHKVYACE VTHQGLSSPV |
| | | TKSFNRGEC |

In some instances, the heavy chain polypeptide sequence for the anti-BCMA arm of elranatamab may lack the C-terminal lysine residue that is present in SEQ ID NO: 50. In some instances, the heavy chain polypeptide sequence for the anti-CD3 arm of elranatamab may lack the C-terminal lysine residue that is present in SEQ ID NO: 63. In some instances, the heavy chain polypeptide sequence for the anti-BCMA arm of elranatamab may lack the C-terminal lysine residue that is present in SEQ ID NO: 50 and the heavy chain polypeptide sequence for the anti-CD3 arm of elranatamab may lack the C-terminal lysine residue that is present in SEQ ID NO: 63.

In some instances, the anti-FcRH5/anti-CD3 bispecific antibody and/or the anti-BCMA/anti-CD3 bispecific antibody according to any of the above embodiments described above may incorporate any of the features, singly or in combination, as described in Sections 1-7 below.

### 1. Antibody Affinity

In certain embodiments, an antibody provided herein has a dissociation constant (KD) of ≤1 μM, ≤250 nM, ≤100 nM, ≤15 nM, ≤10 nM, ≤6 nM, ≤4 nM, ≤2 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one embodiment, KD is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 pl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, KD is measured using a BIACORE® surface plasmon resonance assay. For example, an assay using a BIACORE®-2000 or a BIACORE®-3000 (BIAcore, Inc., Piscataway, NJ) is performed at 37° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 pM) before injection at a flow rate of 5 pl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 37° C. at a flow rate of approximately 25 pl/min. Association rates (kon, or ka) and dissociation rates (koff, or kd) are calculated using a simple one-to-one Langmuir binding model (BIACORE @Evaluation Software version 3.2) by simultaneously fitting the association and dissociation sensorgrams. The equilibrium dissociation constant (KD) is calculated as the ratio koff/kon. See, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 37° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### 2. Antibody Fragments

In certain embodiments, an antibody provided herein (e.g., an anti-FcRH5/anti-CD3 antibody) is an antibody fragment that specifically binds FcRH5 and CD3. In certain embodiments, an antibody provided herein (e.g., an anti-BCMA/anti-CD3 antibody) is an antibody fragment that specifically binds BCMA and CD3. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain embodiments, an antibody provided herein (e.g., an anti-FcRH5/anti-CD3 antibody or an anti-BCMA/anti-CD3 antibody) is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof), for example, are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein (e.g., an anti-FcRH5/anti-CD3 antibody or an anti-BCMA/anti-CD3 antibody) is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Multispecific Antibodies

In any one of the above aspects, an anti-FcRH5/anti-CD3 antibody or an anti-BCMA/anti-CD3 antibody provided herein is a multispecific antibody, for example, a bispecific antibody. Multispecific antibodies are antibodies (e.g., monoclonal antibodies) that have binding specificities for at least two different sites, e.g., antibodies having binding specificities for an immune effector cell and for a cell surface antigen (e.g., a tumor antigen, e.g., FcRH5) on a target cell other than an immune effector cell. In some aspects, one of the binding specificities is for FcRH5 and the other is for CD3. In some aspects, one of the binding specificities is for BCMA and the other is for CD3.

In some aspects, the cell surface antigen may be expressed in low copy number on the target cell. For example, in some aspects, the cell surface antigen is expressed or present at less than 35,000 copies per target cell. In some embodiments, the low copy number cell surface antigen is present between 100 and 35,000 copies per target cell; between 100 and 30,000 copies per target cell; between 100 and 25,000 copies per target cell; between 100 and 20,000 copies per target cell; between 100 and 15,000 copies per target cell; between 100 and 10,000 copies per target cell; between 100 and 5,000 copies per target cell; between 100 and 2,000 copies per target cell; between 100 and 1,000 copies per target cell; or between 100 and 500 copies per target cell. Copy number of the cell surface antigen can be determined, for example, using a standard Scatchard plot.

In some embodiments, a bispecific antibody may be used to localize a cytotoxic agent to a cell that expresses a tumor antigen, e.g., FcRH5 or BCMA. Bispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). “Knob-in-hole” engineering of multispecific antibodies may be utilized to generate a first arm containing a knob and a second arm containing the hole into which the knob of the first arm may bind. The knob of the multispecific antibodies disclosed herein may be an anti-CD3 arm in one embodiment. Alternatively, the knob of the multispecific antibodies disclosed herein may be an anti-target/antigen (e.g., FcRH5 or BCMA) arm in one embodiment. The hole of the multispecific antibodies disclosed herein may be an anti-CD3 arm in one embodiment. Alternatively, the hole of the multispecific antibodies disclosed herein may be an anti-target/antigen arm (e.g., FcRH5 or BCMA) in one embodiment.

Multispecific antibodies may also be engineered using immunoglobulin crossover (also known as Fab domain exchange or CrossMab format) technology (see, e.g., WO2009/080253; Schaefer et al., Proc. Natl. Acad. Sci. USA, 108:11187-11192 (2011)). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibodies, or antibody fragments thereof, may also include a “Dual Acting FAb” or “DAF” comprising an antigen binding site that binds to CD3 as well as another, different antigen (e.g., a second biological molecule) (see, e.g., US 2008/0069820).

### 6. Antibody Variants

In some aspects, amino acid sequence variants of the bispecific anti-FcRH5/anti-CD3 antibodies or bispecific anti-BCMA/anti-CD3 antibodies disclosed herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

a. Substitution, Insertion, and Deletion Variants

In certain embodiments, antibody variants having one or more amino acid substitutions are provided. Sites of interest for substitutional mutagenesis include the CDRs and FRs. Conservative substitutions are shown in Table 4 under the heading of “preferred substitutions.” More substantial changes are provided in Table 4 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 4
Exemplary and Preferred Amino Acid Substitutions

| Original | Exemplary | Preferred |
|---|---|---|
| Residue | Substitutions | Substitutions |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact an antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

b. Glycosylation Variants

In certain embodiments, bispecific anti-FcRH5/anti-CD3 antibodies or bispecific anti-BCMA/anti-CD3 antibodies disclosed herein can be altered to increase or decrease the extent to which the antibody is glycosylated. Addition or deletion of glycosylation sites to anti-FcRH5/anti-CD3 or anti-BCMA/anti-CD3 antibody of the disclosure may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, bispecific anti-FcRH5/anti-CD3 antibody variants or bispecific anti-BCMA/anti-CD3 antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Bispecific anti-FcRH5/anti-CD3 antibody variants or bispecific anti-BCMA/anti-CD3 antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c. Fc Region Variants

In certain embodiments, one or more amino acid modifications may be introduced into the Fc region of a bispecific anti-FcRH5/anti-CD3 antibody or a bispecific anti-BCMA/anti-CD3 antibody, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the disclosure contemplates a bispecific anti-FcRH5/anti-CD3 antibody variant or a bispecific anti-BCMA/anti-CD3 antibody variant that possesses some but not all effector functions, which make it a desirable candidate for applications in which the half-life of the antibody in vivo is important, yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, et al. Proc. Nat'l Acad. Sci. USA 83:7059-7063 (1986)) and Hellstrom et al., Proc. Nat'l Acad. Sci. USA 82:1499-1502 (1985); 5,821,337 (see Bruggemann et al., J. Exp. Med. 166:1351-1361 (1987)). Alternatively, non-radioactive assays methods may be employed (see, for example, ACTITM non-radioactive cytotoxicity assay for flow cytometry (CellTechnology, Inc. Mountain View, CA; and CYTOTOX 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg et al. Blood. 101:1045-1052 (2003); and Cragg et al. Blood. 103:2738-2743 (2004)). FcRn binding and in vivo clearance/half-life determinations can also be performed using methods known in the art (see, e.g., Petkova et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain embodiments, the proline at position 329 of a wild-type human Fc region in the antibody is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcγRIII (Sondermann et al. Nature. 406, 267-273, 2000). In certain embodiments, the antibody comprises at least one further amino acid substitution. In one embodiment, the further amino acid substitution is S228P, E233P, L234A, L235A, L235E, N297A, N297D, or P331S, and still in another embodiment the at least one further amino acid substitution is L234A and L235A of the human IgG1 Fc region or S228P and L235E of the human IgG4 Fc region (see e.g., US 2012/0251531), and still in another embodiment the at least one further amino acid substitution is L234A and L235A and P329G of the human IgG1 Fc region.

Certain antibody variants with improved or diminished binding to FcRs are described. See, e.g., U.S. Pat. No. 6,737,056; WO 2004/056312, and Shields et al., J. Biol. Chem. 9(2): 6591-6604 (2001).

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects, the bispecific anti-FcRH5/anti-CD3 antibody comprises an Fc region comprising an N297G mutation (EU numbering). In some aspects, the anti-FcRH5 arm of the bispecific antibody comprises a N297G mutation and/or the anti-CD3 arm of the bispecific antibody comprises an Fc region comprising an N297G mutation.

In some embodiments, the bispecific anti-FcRH5/anti-CD3 antibody comprising the N297G mutation comprises an anti-FcRH5 arm comprising a first binding domain comprising the following six HVRs (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6; and an anti-CD3 arm comprising an N297G mutation. In some embodiments, the anti-CD3 arm comprising the N297G mutation comprises the following six HVRs: (a) an HVR-H1 comprising the amino acid sequence of SEQ ID NO: 9; (b) an HVR-H2 comprising the amino acid sequence of SEQ ID NO: 10; (c) an HVR-H3 comprising the amino acid sequence of SEQ ID NO: 11; (d) an HVR-L1 comprising the amino acid sequence of SEQ ID NO: 12; (e) an HVR-L2 comprising the amino acid sequence of SEQ ID NO: 13; and (f) an HVR-L3 comprising the amino acid sequence of SEQ ID NO: 14.

In some embodiments, the bispecific anti-FcRH5/anti-CD3 antibody comprising the N297G mutation comprises an anti-FcRH5 arm comprising a first binding domain comprising (a) a VH domain comprising an amino acid sequence of SEQ ID NO: 7 and (b) a VL domain comprising an amino acid sequence of SEQ ID NO: 8, and an anti-CD3 arm comprising an N297G mutation. In some embodiments, the anti-CD3 arm comprising the N297G mutation comprises comprising (a) a VH domain comprising an amino acid sequence of SEQ ID NO: 15 and (b) a VL domain comprising an amino acid sequence of SEQ ID NO: 16.

In some embodiments, the bispecific anti-FcRH5/anti-CD3 antibody comprising the N297G mutation comprises one or more heavy chain constant domains, wherein the one or more heavy chain constant domains are selected from a first CH1 (CH11) domain, a first CH2 (CH21) domain, a first CH3 (CH31) domain, a second CH1 (CH12) domain, second CH2 (CH22) domain, and a second CH3 (CH32) domain. In some aspects, at least one of the one or more heavy chain constant domains is paired with another heavy chain constant domain. In some aspects, the CH31 and CH32 domains each comprise a protuberance or cavity, and wherein the protuberance or cavity in the CH31 domain is positionable in the cavity or protuberance, respectively, in the CH32 domain. In some aspects, the CH31 and CH32 domains meet at an interface between said protuberance and cavity. In some aspects, the CH21 and CH22 domains each comprise a protuberance or cavity, and wherein the protuberance or cavity in the CH21 domain is positionable in the cavity or protuberance, respectively, in the CH22 domain. In other instances, the CH21 and CH22 domains meet at an interface between said protuberance and cavity. In some aspects, the anti-FcRH5 antibody is an IgG1 antibody.

In some embodiments, the bispecific anti-FcRH5/anti-CD3 antibody comprising the N297G mutation comprises an anti-FcRH5 arm comprising a first binding domain comprising (a) a VH domain comprising the amino acid sequence of SEQ ID NO: 7 and (b) a VL domain comprising the amino acid sequence of SEQ ID NO: 8, and an anti-CD3 arm, wherein (a) the anti-FcRH5 arm comprises T366S, L368A, Y407V, and N297G amino acid substitution mutations (EU numbering) and (b) the anti-CD3 arm comprises T366W and N297G substitution mutations (EU numbering). In some embodiments, the anti-CD3 arm comprising the T366W and N297G mutations comprises comprising (a) a VH domain comprising an amino acid sequence of SEQ ID NO: 15 and (b) a VL domain comprising an amino acid sequence of SEQ ID NO: 16.

In other embodiments, the bispecific anti-FcRH5/anti-CD3 antibody comprising the N297G mutation comprises an anti-FcRH5 arm comprising a first binding domain comprising (a) a VH domain comprising an amino acid sequence of SEQ ID NO: 7 and (b) a VL domain comprising an amino acid sequence of SEQ ID NO: 8, and an anti-CD3 arm, wherein (a) the anti-FcRH5 arm comprises T366W and N297G substitution mutations (EU numbering) and (b) the anti-CD3 arm comprises T366S, L368A, Y407V, and N297G mutations (EU numbering). In some embodiments, the anti-CD3 arm comprising the N297G mutation comprises comprising (a) a VH domain comprising an amino acid sequence of SEQ ID NO: 15 and (b) a VL domain comprising an amino acid sequence of SEQ ID NO: 16.

In some embodiments, the bispecific anti-BCMA/anti-CD3 antibody comprises an anti-BCMA arm comprising a first binding domain comprising a VH domain comprising an amino acid sequence of SEQ ID NO: 48 and a VL domain comprising an amino acid sequence of SEQ ID NO: 49; and an anti-CD3 arm comprising a second binding domain comprising a VH domain comprising an amino acid sequence of SEQ ID NO: 61 and a VL domain comprising an amino acid sequence of SEQ ID NO: 62, wherein the bispecific anti-BCMA/anti-CD3 antibody comprises first and second respective human IgG2 constant regions comprising amino acid modifications at positions 223 and 228, or 223, 225 and 228, respectively, in the hinge region, and at position 368 or 409, respectively, in the CH3 region (EU numbering). In some embodiments, the first constant region comprises C223E, P228E and L368E (EU numbering). In some embodiments, the second constant region comprises C223R, E225R, P228R and K409R (EU numbering).

For example, in some embodiments, the bispecific anti-BCMA/anti-CD3 antibody comprises an anti-BCMA arm comprising a first binding domain comprising a VH domain comprising an amino acid sequence of SEQ ID NO: 48 and a VL domain comprising an amino acid sequence of SEQ ID NO: 49; and an anti-CD3 arm comprising a second binding domain comprising a VH domain comprising an amino acid sequence of SEQ ID NO: 61 and a VL domain comprising an amino acid sequence of SEQ ID NO: 62, wherein the bispecific anti-BCMA/anti-CD3 antibody comprises first and second respective human IgG2 constant regions, wherein the first constant region comprises C223E, P228E and L368E (EU numbering). In some embodiments, the second constant region comprises C223R, E225R, P228R and K409R (EU numbering).

In some embodiments, the bispecific anti-BCMA/anti-CD3 antibody comprises an anti-BCMA arm comprising a first binding domain comprising a VH domain comprising an amino acid sequence of SEQ ID NO: 48 and a VL domain comprising an amino acid sequence of SEQ ID NO: 49; and an anti-CD3 arm comprising a second binding domain comprising a VH domain comprising an amino acid sequence of SEQ ID NO: 61 and a VL domain comprising an amino acid sequence of SEQ ID NO: 62, wherein the bispecific anti-BCMA/anti-CD3 antibody comprises first and second respective human IgG2 constant regions, wherein the second constant region comprises C223R, E225R, P228R and K409R (EU numbering). In some embodiments, the first constant region comprises C223E, P228E and L368E (EU numbering).

In some embodiments, the bispecific anti-BCMA/anti-CD3 antibody comprises an anti-BCMA arm comprising a first binding domain comprising a VH domain comprising an amino acid sequence of SEQ ID NO: 48 and a VL domain comprising an amino acid sequence of SEQ ID NO: 49; and an anti-CD3 arm comprising a second binding domain comprising a VH domain comprising an amino acid sequence of SEQ ID NO: 61 and a VL domain comprising an amino acid sequence of SEQ ID NO: 62, wherein the bispecific anti-BCMA/anti-CD3 antibody comprises first and second respective human IgG2 constant regions, wherein the first constant region comprises C223E, P228E and L368E (EU numbering) and the second constant region comprises C223R, E225R, P228R and K409R (EU numbering).

d. Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

### 7. Charged Regions

In some aspects, the binding domain that specifically binds FcRH5 or CD3 comprises a VH1 comprising a charged region (CR1) and a VL1 comprising a charged region (CR2), wherein the CR1 in the VH1 forms a charge pair with the CR2 in the VL1. In some aspects, the CR1 comprises a basic amino acid residue and the CR2 comprises an acidic amino acid residue. In some aspects, the CR1 comprises a Q39K substitution mutation (Kabat numbering). In some aspects, the CR1 consists of the Q39K substitution mutation. In some aspects, the CR2 comprises a Q38E substitution mutation (Kabat numbering). In some aspects, the CR2 consists of the Q38E substitution mutation. In some aspects, the second binding domain that binds CD3 comprises a VH2 comprising a charged region (CR3) and a VL2 comprising a charged region (CR4), wherein the CR4 in the VL2 forms a charge pair with the CR3 in the VH2. In some aspects, the CR4 comprises a basic amino acid residue and the CR3 comprises an acidic amino acid residue. In some aspects, the CR4 comprises a Q38K substitution mutation (Kabat numbering). In some aspects, the CR4 consists of the Q38K substitution mutation. In some aspects, the CR3 comprises a Q39E substitution mutation (Kabat numbering). In some aspects, the CR3 consists of the Q39E substitution mutation. In some aspects, the VL1 domain is linked to a light chain constant domain (CL1) domain and the VH1 is linked to a first heavy chain constant domain (CH1), wherein the CL1 comprises a charged region (CR5) and the CH1 comprises a charged region (CR6), and wherein the CR5 in the CL1 forms a charge pair with the CR6 in the CH11. In some aspects, the CR5 comprises a basic amino acid residue and the CR6 comprises an acidic residue. In some aspects, the CR5 comprises a V133K substitution mutation (EU numbering). In some aspects, the CR5 consists of the V133K substitution mutation. In some aspects, the CR6 comprises a S183E substitution mutation (EU numbering). In some aspects, the CR6 consists of the S183E substitution mutation.

In other aspects, the VL2 domain is linked to a CL domain (CL2) and the VH2 is linked to a CH1 domain (CH12), wherein the CL2 comprises a charged region (CR7) and the CH12 comprises a charged region (CR8), and wherein the CR8 in the CH12 forms a charge pair with the CR7 in the CL2. In some aspects, the CR8 comprises a basic amino acid residue and the CR7 comprises an acidic amino acid residue. In some aspects, the CR8 comprises a S183K substitution mutation (EU numbering). In some aspects, the CR8 consists of the S183K substitution mutation. In some aspects, the CR7 comprises a V133E substitution mutation (EU numbering). In some aspects, the CR7 consists of the V133E substitution mutation.

In other aspects, the VL2 domain is linked to a CL domain (CL2) and the VH2 is linked to a CH1 domain (CH12), wherein (a) the CL2 comprises one or more mutations at amino acid residues F116, L135, S174, S176, and/or T178 (EU numbering) and (b) the CH12 comprises one or more mutations at amino acid residues A141, F170, S181, S183, and/or V185 (EU numbering). In some aspects, the CL2 comprises one or more of the following substitution mutations: F116A, L135V, S174A, S176F, and/or T178V. In some aspects, the CL2 comprises the following substitution mutations: F116A, L135V, S174A, S176F, and T178V. In some aspects, the CH12 comprises one or more of the following substitution mutations: A141I, F170S, S181M, S183A, and/or V185A. In some aspects, the CH12 comprises the following substitution mutations: A141I, F170S, S181M, S183A, and V185A.

In other aspects, the binding domain that specifically binds FcRH5 or CD3 comprises a VH domain (VH1) comprising a charged region (CR1) and a VL domain (VL1) comprising a charged region (CR2), wherein the CR2 in the VL1 forms a charge pair with the CR1 in the VH1. In some aspects, the CR2 comprises a basic amino acid residue and the CR1 comprises an acidic amino acid residue. In some aspects, the CR2 comprises a Q38K substitution mutation (Kabat numbering). In some aspects, the CR2 consists of the Q38K substitution mutation. In some aspects, the CR1 comprises a Q39E substitution mutation (Kabat numbering). In some aspects, the CR1 consists of the Q39E substitution mutation. In some aspects, the second binding domain that binds CD3 comprises a VH domain (VH2) comprising a charged region (CR3) and a VL domain (VL2) comprising a charged region (CR4), wherein the CR3 in the VH2 forms a charge pair with the CR4 in the VL2. In some aspects, the CR3 comprises a basic amino acid residue and the CR4 comprises an acidic amino acid residue. In some aspects, the CR3 comprises a Q39K substitution mutation (Kabat numbering). In some aspects, the CR3 consists of the Q39K substitution mutation. In some aspects, the CR4 comprises a Q38E substitution mutation (Kabat numbering). In some aspects, the CR4 consists of the Q38E substitution mutation. In some aspects, the VL1 domain is linked to a light chain constant domain (CL1) and the VH1 is linked to a first heavy chain constant domain (CH1 1), wherein the CL1 comprises a charged region (CR5) and the CH11 comprises a charged region (CR6), and wherein the CR6 in the CH11 forms a charge pair with the CR5 in the CL1. In some aspects, the CR6 comprises a basic amino acid residue and the CR5 comprises an acidic amino acid residue. In some aspects, the CR6 comprises a S183K substitution mutation (EU numbering). In some aspects, the CR6 consists of the S183K substitution mutation. In some aspects, the CR5 comprises a V133E substitution mutation (EU numbering). In some aspects, the CR5 consists of the V133E substitution mutation.

In other aspects, the VL2 domain is linked to a CL domain (CL2) and the VH2 is linked to a CH1 domain (CH12), wherein the CL2 comprises a charged region (CR7) and the CH12 comprises a charged region (CR8), and wherein the CR7 in the CL2 forms a charged pair with the CR8 in the CH12. In some aspects, the CR7 comprises a basic amino acid residue and the CR8 comprises an acidic residue. In some aspects, the CR7 comprises a V133K substitution mutation (EU numbering). In some aspects, the CR7 consists of the V133K substitution mutation. In some aspects, the CR8 comprises a S183E substitution mutation (EU numbering). In some aspects, the CR8 consists of the S183E substitution mutation.

In other aspects, the VL2 domain is linked to a CL domain (CL2) and the VH2 is linked to a CH1 domain (CH12), wherein (a) the CL2 comprises one or more mutations at amino acid residues F116, L135, S174, S176, and/or T178 (EU numbering) and (b) the CH12 comprises one or more mutations at amino acid residues A141, F170, S181, S183, and/or V185 (EU numbering). In some aspects, the CL2 comprises one or more of the following substitution mutations: F116A, L135V, S174A, S176F, and/or T178V. In some aspects, the CL2 comprises the following substitution mutations: F116A, L135V, S174A, S176F, and T178V. In some aspects, the CH12 comprises one or more of the following substitution mutations: A141I, F170S, S181M, S183A, and/or V185A. In some aspects, the CH12 comprises the following substitution mutations: A141I, F170S, S181M, S183A, and V185A. In some aspects, the anti-FcRH5 antibody comprises one or more heavy chain constant domains, wherein the one or more heavy chain constant domains are selected from a first CH2 domain (CH21), a first CH3 domain (CH31), a second CH2 domain (CH22), and a second CH3 domain (CH32). In some aspects, at least one of the one or more heavy chain constant domains is paired with another heavy chain constant domain. In some aspects, the CH31 and the CH32 each comprise a protuberance (P1) or a cavity (C1), and wherein the P1 or the C1 in the CH31 is positionable in the C1 or the P1, respectively, in the CH32. In some aspects, the CH31 and the CH32 meet at an interface between the P1 and the C1. In some aspects, the CH21 and the CH22 each comprise (P2) or a cavity (C2), and wherein the P2 or the C2 in the CH21 is positionable in the C2 or the P2, respectively, in the CH22. In some aspects, the CH21 and the CH22 meet at an interface between the P2 and the C2.

### F. Recombinant Methods and Compositions

Bispecific anti-FcRH5/anti-CD3 and/or anti-BCMA/anti-CD3 antibodies disclosed herein may be produced using recombinant methods and compositions, for example, as described in U.S. Pat. No. 4,816,567. In one embodiment, an isolated nucleic acid encoding an anti-FcRH5 antibody described herein is provided. In one embodiment, an isolated nucleic acid encoding an anti-BCMA antibody described herein is provided. Such nucleic acid may encode an amino acid sequence comprising the VL and/or an amino acid sequence comprising the VH of the antibody (e.g., the light and/or heavy chains of the antibody). In another embodiment, an isolated nucleic acid encoding an anti-CD3 antibody described herein is provided. Such a nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such a nucleic acid are provided. In a further embodiment, a host cell comprising such a nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making a bispecific anti-FcRH5/anti-CD3 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of a bispecific anti-FcRH5/anti-CD3 antibody or a bispecific anti-BCMA/anti-CD3 antibody, a nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

### 1. Two-Cell Methods for Manufacturing Bispecific Antibodies

In some aspects, an antibody disclosed herein (e.g., a bispecific anti-FcRH5/anti-CD3 antibody or a bispecific anti-BCMA/anti-CD3 antibody) is manufactured using a method comprising two host cell lines. In some aspects, a first arm of a bispecific antibody (e.g., a first arm comprising a hole region) is produced in a first host cell line, and a second arm of the antibody (e.g., a second arm comprising a knob region) is produced in a second host cell line. The arms of the antibody are purified from the host cell lines and assembled in vitro.

### 2. One-Cell Methods for Manufacturing Bispecific Antibodies

In some aspects, an antibody disclosed herein (e.g., a bispecific anti-FcRH5/anti-CD3 antibody or a bispecific anti-BCMA/anti-CD3 antibody) is manufactured using a method comprising a single host cell line. In some aspects, a first arm of the antibody (e.g., a first arm comprising a hole region) and a second arm of the antibody (e.g., a second arm comprising a knob region) are produced in and purified from a single host cell line. Preferably, the first arm and the second arm are expressed at comparable levels in the host cell, e.g., are both expressed at a high level in the host cell. Similar levels of expression increase the likelihood of efficient TDB production and decrease the likelihood of light chain (LC) mispairing of TDB components. The first arm and second arm of the antibody may each further comprise amino acid substitution mutations introducing charge pairs, as described in Section IIB (7) herein. The charge pairs promote the pairing of heavy and light chain cognate pairs of each arm of the bispecific antibody, thereby minimizing mispairing.

### 3. Host Cells

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### G. Pharmaceutical Compositions and Formulations

Pharmaceutical compositions and formulations of the anti-FcRH5/anti-CD3 bispecific antibodies disclosed herein and/or the anti-BCMA/anti-CD3 bispecific antibodies disclosed herein can be prepared by mixing such antibodies having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as L-Histidine/glacial acetic acid (e.g., at pH 5.8), phosphate, citrate, and other organic acids; tonicity agents, such as sucrose; stabilizers, such as L-methionine; antioxidants including N-acetyl-DL-tryptophan, ascorbic acid, and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polysorbate 20 or polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

### III. Articles of Manufacture

In another aspect of the invention, an article of manufacture containing materials useful for the treatment, prevention, and/or diagnosis of the disorders described above is provided. The article of manufacture may comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an anti-FcRH5/anti-CD3 bispecific antibody described herein and/or an anti-BCMA/anti-CD3 bispecific antibody described herein. In some aspects, the containers (e.g., vials) may be different sizes, e.g., may have sizes proportional to the amount of the composition they contain. Articles of manufacture comprising containers (e.g., vials) proportional to the intended doses may, e.g., increase convenience, minimize waste, and/or increase cost-effectiveness. The label or package insert indicates that the composition is used for treating the condition of choice (e.g., a multiple myeloma (MM)) and further includes information related to at least one of the dosing regimens described herein. Moreover, the article of manufacture may comprise (a) a first container with a composition contained therein, wherein the composition comprises an anti-FcRH5/anti-CD3 bispecific antibody described herein; and (b) a second container with a composition contained therein, wherein the composition comprises an anti-BCMA/anti-CD3 bispecific antibody described herein. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one aspect, provided herein is a kit for treating a subject having a cancer (e.g., a hematologic cancer (e.g., a B cell proliferative disorder (e.g., an MM))), the kit comprising a bispecific antibody that specifically binds to FcRH5 and CD3 (e.g., cevostamab) and instructions to administer the bispecific antibody that specifically binds to FcRH5 and CD3 to the subject in combination with a second bispecific antibody (e.g., an anti-BCMA/anti-CD3 antibody, e.g., elranatamab).

In one aspect, provided herein is a kit for treating a subject having a cancer (e.g., a hematologic cancer (e.g., a B cell proliferative disorder (e.g., an MM))), the kit comprising a bispecific antibody that specifically binds to BCMA and CD3 (e.g., elranatamab) and instructions to administer the bispecific antibody that specifically binds to BCMA and CD3 to the subject in combination with a second bispecific antibody (e.g., an anti-FcRH5/anti-CD3 antibody, e.g., cevostamab).

In another example, provided herein is a kit for treating a subject having an R/R MM, the kit comprising cevostamab and/or elranatamab and instructions to administer to the subject cevostamab and elranatamab in a dosing regimen comprising: (i) a pre-phase comprising an approximately 15-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is a kit for treating a subject having an R/R MM, the kit comprising cevostamab and/or elranatamab and instructions to administer to the subject cevostamab and elranatamab in a dosing regimen comprising: (i) a pre-phase comprising an approximately 22-day dosing cycle (C1); (ii) a first phase, following the pre-phase, comprising a first dosing cycle (C1), wherein the C1 of the first phase is a 14-day dosing cycle; (iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iv) a third phase, following the second phase, comprising one or more dosing cycles, wherein cevostamab is administered to the subject: at a step-up dose of 3.6 mg on Day 16, Day 17, or Day 18 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase; at a target dose of 76 mg on Day 1 of the C1 of the first phase; at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is a kit for treating a subject having an R/R MM, the kit comprising cevostamab and/or elranatamab and instructions to administer to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase.

In another example, provided herein is a kit for treating a subject having an R/R MM, the kit comprising cevostamab and/or elranatamab and instructions to administer to the subject cevostamab and elranatamab in a dosing regimen comprising: (i) a first phase comprising a first dosing cycle (C1), wherein the C1 of the first phase is a 14-day dosing cycle; (ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and (iii) a third phase, following the second phase, comprising one or more dosing cycles, wherein cevostamab is administered to the subject: at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject: at a target dose of 76 mg on Day 1 and Day 8 of the C1 of the first phase; at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

In another example, provided herein is a kit comprising a first bispecific antibody that specifically binds to FcRH5 and CD3 for use in treatment of a subject having an MM, the treatment comprising administration of the first bispecific antibody and a second bispecific antibody that specifically binds to BCMA and CD3 to the subject.

### EXAMPLES

The following are examples of the methods of the invention. It is understood that various other embodiments may be practiced, given the general description provided above.

### Example 1. An Open-Label, Multicenter, Phase Ib Trial Evaluating the Safety, Pharmacokinetics, and Activity of the Combination of Cevostamab and Elranatamab in Patients with Relapsed or Refractory Multiple Myeloma

GO43979 is an open-label, multicenter, phase Ib trial designed to evaluate the safety, tolerability, pharmacokinetics, and activity of cevostamab in combination with elranatamab in patients with relapsed/refractory multiple myeloma (R/R MM). Participants will receive cevostamab intravenously (IV) in combination with elranatamab subcutaneously (SC), with step-up dosing of each drug in pre-phase following which they will receive elranatamab, at the assigned dose as a SC injection until disease progression or unacceptable toxicity. Participants will also receive cevostamab at the assigned dose as IV infusion until disease progression or unacceptable toxicity or up to 1 year on treatment, whichever occurs first. Tocilizumab will be used as rescue medication for participants who experience a cytokine release syndrome (CRS) event.

Specific objectives and corresponding endpoints for the study are outlined in Table 5, below.

“Study treatment” refers to the combination of treatments assigned to patients as part of this study (i.e., cevostamab and elranatamab).

TABLE 5
Primary and Secondary Objectives and Endpoints

| | Corresponding Endpoints |
|---|---|
| Primary Objective | |
| To evaluate safety and | Incidence and severity of adverse events (including dose- |
| tolerability of the combination | limiting toxicities (DLTs)) and laboratory abnormalities, |
| of cevostamab plus elranatamab | with severity determined according to National Cancer |
| | Institute Common Terminology Criteria for Adverse |
| | Events (NCI CTCAE) v5.0 for the majority of adverse |
| | events and laboratory abnormalities; American Society |
| | of Transplantation and Cellular Therapy (ASTCT) |
| | Consensus Grading for the severity of cytokine release |
| | syndrome (CRS) (Lee et al., Biol Blood Marrow |
| | Transplant, 25: 625-638, 2019); and NCI CTCAE and |
| | ASTCT Consensus Grading for the severity of immune |
| | effector cell-associated neurotoxicity syndrome |
| | (ICANS). |
| To identify a recommended | Relationship between cevostamab plus elranatamab |
| Phase II dose (RP2D) for the | doses and safety, pharmacokinetic (PK), and |
| study treatment. | immunogenicity endpoints. |
| Secondary Objectives | |
| To make a preliminary | Note: International Myeloma Working Group (IMWG) |
| assessment of the activity the | criteria are used for the following endpoints. |
| study treatment. | Objective response rate (ORR) as determined by the |
| | investigator. |
| | ORR is defined as the proportion of patients with |
| | a stringent complete response (sCR), complete |
| | response (CR), very good partial response |
| | (VGPR), or partial response (PR) on two |
| | consecutive occasions. |
| | CR/sCR rate, as determined by the investigator. |
| | CR/sCR rate is defined as proportion of patients |
| | achieving a CR or sCR on two consecutive |
| | occasions. |
| | Rate of VGPR or better, as determined by the |
| | investigator. |
| | Rate of VGPR or better is defined as the |
| | proportion of patients achieving a VGPR or |
| | better on two consecutive occasions. |
| | Progression-free survival (PFS), as determined by |
| | the investigator. |
| | PFS is defined as the time from initiation of |
| | study treatment to the first date of disease |
| | progression or death from any cause, whichever |
| | occurs first. |
| | Duration of response (DOR), as determined by the |
| | investigator (for patients who achieve a response of |
| | PR or better). |
| | DOR is defined as the number of days from the |
| | date of first documented response of PR or better |
| | until date of disease progression or death from |
| | any cause, whichever occurs first. |
| | Time to first response (for patients who achieve a |
| | response of PR or better). |
| | Time to first response is defined as time from |
| | initiation of study treatment to achieving a |
| | confirmed PR or better. |
| | Time to best response (for patients who achieve a |
| | response of PR or better). |
| | Time to best response is defined as time from |
| | initiation of study treatment to achieving the |
| | deepest response. |
| | Overall survival (OS), defined as the time from |
| | initiation of study treatment to death from any cause. |
| To characterize the | Serum concentration of cevostamab at specified |
| pharmacokinetics of | timepoints. |
| cevostamab and elranatamab | Serum concentration of elranatamab at specified |
| when given in | timepoints. |
| combination. | Pharmacokinetic (PK) parameters of cevostamab. |
| | PK parameters of elranatamab. |
| To evaluate the immune | Prevalence of anti-drug antibodies (ADAs) against |
| response to cevostamab and | cevostamab and elranatamab at baseline and incidence |
| elranatamab when given in | of ADAs against cevostamab and elranatamab during |
| combination. | the study. |
| Exploratory Objectives | |
| To evaluate potential | Relationship between serum concentration or PK |
| relationships between the | parameters for cevostamab and elranatamab, and |
| exposure of cevostamab in | pharmacodynamic biomarkers, including but not |
| combination with elranatamab | limited to cytokine release, T-cell number, and T-cell |
| and pharmacodynamic | activation state. |
| biomarkers. | |
| To evaluate potential | Relationship between selected covariates and serum |
| relationships between selected | concentration or PK parameters for cevostamab and |
| covariates and exposure to | elranatamab. |
| cevostamab and elranatamab. | |
| To evaluate potential effects of | Relationship between status of ADAs against |
| ADAs against cevostamab and | cevostamab and elranatamab, and safety, PK, |
| elranatamab. | pharmacodynamic (PD), or activity endpoints. |
| To evaluate the activity of | Minimal residual disease (MRD) negativity at time of |
| cevostamab in combination | first CR. |
| with elranatamab in driving | |
| MRD negatively in patients | |
| with R/R MM. | |
| To identify biomarkers that may | Relationship between biomarkers in blood and tumor |
| be: | tissue and activity, safety, PK, immunogenicity, or |
| Able to provide evidence of | other biomarker endpoints. |
| cevostamab activity in | |
| combination with elranatamab. | |
| Associated with response to, | |
| resistance to, or progression | |
| with cevostamab in | |
| combination with elranatamab. | |
| Associated with susceptibility to | |
| developing adverse events (i.e., | |
| safety biomarkers). | |
| To make a preliminary as- | Outcome of CRS following the administration of |
| sessment of the efficacy of | tocilizumab. |
| tocilizumab (ACTEMRA ®/ | |
| ROACTEMRA ®) in | |
| ameliorating the symptoms of | |
| CRS following administration | |
| of study treatment. | |

### A. Overall Design and Study Population

The study evaluates the safety, pharmacokinetics, and activity of cevostamab plus elranatamab in patients with R/R MM per International Myeloma Working Group (IMWG) The study consists of a dose-limiting toxicity (DLT) assessment stage, a safety lead-in stage, and an expansion stage. Several key aspects of the study design and study population are summarized below.

### Study Treatment

### Initial Study Treatment Dosing Schedule (Option 1)

Elranatamab (administered subcutaneously (SC)):
Pre-phase (about 15 days): 12 mg (step-up dose) on Day 1; 32 mg (step-up dose) on Day 4; 76 mg (target dose) on Day 8.
Cycles 1-26: 76 mg on Day 1 of each 14-day cycle.
Cycles 27 and beyond: 76 mg on Day 1 of every other 14-day cycle (every 28 days). Cevostamab (administered intravenously (IV)):
Pre-phase (about 15 days): 3.6 mg (step-up dose) on Day 9-11.
Cycle 1: 60 mg (target dose) on Day 2-4.
Cycles 2-26: 60 mg on Day 1 of each 14-day cycle (stopped after 26 cycles).
   Study Treatment Dosing Schedule with Pre-Phase Mitigation (Option 2)

### Elranatamab (SC):

Pre-phase (about 22 days): 12 mg on Day 1; 32 mg on Day 3-5; 76 mg on Days 8 and 15.
Cycles 1 and beyond: 76 mg on Day 1 of each 14-day cycle.

### Cevostamab (IV):

Pre-phase (about 22 days): 3.6 mg on Day 16-18.
Cycle 1: 60 mg on Day 2-4.
Cycles 2-26: 60 mg on Day 1 of each 14-day cycle (stopped after 26 cycles).
   Target Dose Level a Dosing Schedule (after about 15-Day or about 22-Day Pre-Phase)

### Elranatamab (SC):

Cycles 1 and beyond: 76 mg on Days 1 of each 14-day cycle.

### Cevostamab (IV):

Cycle 1: 60 mg on Day 2-4.
Cycles 2-26: 60 mg on Day 1 of each 14-day cycle (stopped after 26 cycles).
   Target Dose Level B Dosing Schedule (after −15 or −22-Day Pre-Phase)

### Elranatamab (SC):

Cycles 1 and beyond: 76 mg on Days 1 and 8 of each 14-day cycle.

### Cevostamab (IV):

Cycle 1: 132 mg on Day 2-4.
Cycles 2-26: 132 mg on Day 1 of each 14-day cycle (stopped after 26 cycles).
   Target Dose Level C Dosing Schedule (after −15 or −22-Day Pre-Phase)

### Elranatamab (SC):

Cycles 1 and beyond: 76 mg on Days 1 and 8 of each 14-day cycle.

### Cevostamab (IV):

Cycle 1: 60 mg on Day 2-4.
Cycles 2-26: 60 mg on Day 1 of each 14-day cycle (stopped after 26 cycles).
   Target Dose Level D (Alternating) Dosing Schedule with Target Dose Mitigation (after about 15-Day or about 22-Day Pre-Phase)

### Elranatamab (SC):

Cycle 1: 76 mg on Days 1 and 8.
Cycles 2-26: 76 mg on Day 8 of each 14-day cycle.
Cycles 27 and beyond: 76 mg on Day 1 of each 14-day cycle.

### Cevostamab (IV):

Cycle 1: 60 mg on Day 2-4.
Cycles 2-26: 60 mg on Day 1 of each 14-day cycle (stopped after 26 cycles).
   Target Dose Level E Dosing Schedule (after about 15-Day or about 22-Day Pre-Phase)

### Elranatamab (SC):

Cycle 1: 76 mg on Day 1.
Cycles 2-26: 76 mg on Day 1 of each 14-day cycle.
Cycles 27 and beyond: 76 mg on Day 1 of each 14-day cycle.

### Cevostamab (IV):

Cycle 1: 105 mg on Day 2-4.
Cycles 2-26: 105 mg on Day 1 of each 14-day cycle (stopped after 26 cycles).
   Target Dose Level F Dosing Schedule (after about 15-Day or about 22-Day Pre-Phase)

### Elranatamab (SC):

Cycle 1: 76 mg on Day 1.
Cycles 2-7: 76 mg on Day 1 of each 14-day cycle.
Cycles 8-26: 76 mg on Day 1 of each even numbered 14-day cycle (i.e., 4 weeks).
Cycles 27 and beyond: 76 mg on Day 1 of each even numbered 14-day cycle (i.e., 4 weeks).

### Cevostamab (IV):

Cycle 1: 60 mg on Day 2-4.
Cycles 2-7: 60 mg on Day 1 of each 14-day cycle.
Cycles 8-26: 60 mg on Day 1 of each even numbered 14-day cycle (i.e., 4 weeks) (stopped after 26 cycles).
   Target Dose Level G Dosing Schedule (after about 15-Day or about 22-Day Pre-Phase)

### Elranatamab (SC):

Cycle 1: 76 mg on Day 1.
Cycles 2-7: 76 mg on Day 1 of each 14-day cycle.
Cycles 8-27: 76 mg on Day 1 of each even numbered 14-day cycle (i.e., 4 weeks).
Cycles 27 and beyond: 76 mg on Day 1 of each even numbered 14-day cycle (i.e., 4 weeks).

### Cevostamab (IV):

Cycle 1: 105 mg on Day 2-4.
Cycles 2-7: 105 mg on Day 1 of each 14-day cycle.
Cycles 8-26: 105 mg on Day 1 of each even numbered 14-day cycle (i.e., 4 weeks) (stopped after 26 cycles).

### Tocilizumab (if Necessary):

For all patients, tocilizumab should be administered when indicated as described in the protocol for treatment-emergent CRS.

### Duration of Participation

Cevostamab treatment continues until disease progression per IMWG Criteria, unacceptable toxicity, or up to 26 cycles on treatment, whichever occurs first. Elranatamab treatment continues until disease progression per IMWG Criteria or unacceptable toxicity. The total duration of study participation for each patient is expected to range from 1 day to more than 60 months.

### Study Rationale and Benefit-Risk Assessment

The scientific rationale for the combination of elranatamab and cevostamab is that the combination of two TDBs with different targets may result in enhanced clinical activity by increasing clinical response rates and/or inducing deeper and more durable response in patients with MM. Data from the ongoing clinical trials of both elranatamab and cevostamab have demonstrated that both molecules are able to induce rapid, deep responses in heavily pretreated R/R MM patients. However, not all patients achieve a clinical response, and relapse is expected in the majority of patients. The anti-myeloma activity of both molecules is dependent on engagement of the tumor antigen BCMA or FcRH5 for elranatamab and cevostamab, respectively, and thus tumor antigen heterogeneity or loss of expression may limit the clinical activity of either molecule when given alone.

### Example 2. Study Design

### A. Overall Design

As described in Example 1, the GO43979 study is a Phase Ib, multicenter, open-label study designed to evaluate safety, tolerability, pharmacokinetics, and activity of cevostamab plus elranatamab in patients with R/R MM. The study consists of an initial 3+3 patient dosing cohort during the dose-limiting toxicity (DLT) assessment stage; if there are no DLTs experienced, the cohort is backfilled up to 10 patients. This is followed by safety lead-in cohorts and randomized expansion cohorts to further characterize the safety, pharmacokinetics, and activity of cevostamab in combination with elranatamab to inform the recommended Phase 2 dose (RP2D).

Patients undergo a screening period, a treatment period, and follow-up. Patients receive cevostamab treatment for about 52 weeks or until disease progression, and receive elranatamab treatment until disease progression. There is no intrapatient dose escalation for either study drug.

The screening period lasts up to 28 days for all patients enrolled. Patients who do not meet the criteria for participation in this study (screen failure) may qualify for two re screening opportunities (for a total of three screenings per patient) at the investigator's discretion.

All patients are closely monitored for adverse events throughout the study and for at least 90 days after the last dose of the study treatment. Adverse events and laboratory abnormalities are graded according to the National Cancer Institute Common Terminology Criteria for Adverse Events, Version 5.0 (NCI CTCAE v5.0), with the exception of cytokine release syndrome (CRS), which is graded according to American Society of Transplantation and Cellular Therapy (ASTCT) Consensus Grading for Cytokine Release Syndrome (Lee et al., Biol Blood Marrow Transplant, 25: 625-638, 2019), and immune effector cell-associated neurotoxicity syndrome (ICANS), which will be graded according to ASTCT Consensus Grading and NCI CTCAE v5.0.

Patients with acceptable toxicity and evidence of clinical benefit may continue to receive cevostamab up to Cycle 26 (about 1 year), disease progression (as determined by the investigator according to International Myeloma Working Group (IMWG) criteria), or unacceptable toxicity, whichever occurs first. Patients with acceptable toxicity and evidence of clinical benefit may continue to receive elranatamab until disease progression (as determined by the investigator according to IMWG criteria) or unacceptable toxicity, whichever occurs first. After 6 months of treatment, if a patient achieves a complete response (CR), the elranatamab and cevostamab dose interval will be changed from once every two weeks (Q2W) to once every four weeks (Q4W), starting on the next even-number cycle. If a patient does not achieve a CR but has a partial response (PR) for at least 2 months, the elranatamab dose interval will be changed from Q2W to Q4W starting at Cycle 27 (˜1 year).

Premedication with corticosteroids, antihistamines and anti-pyretics is mandatory (see below).

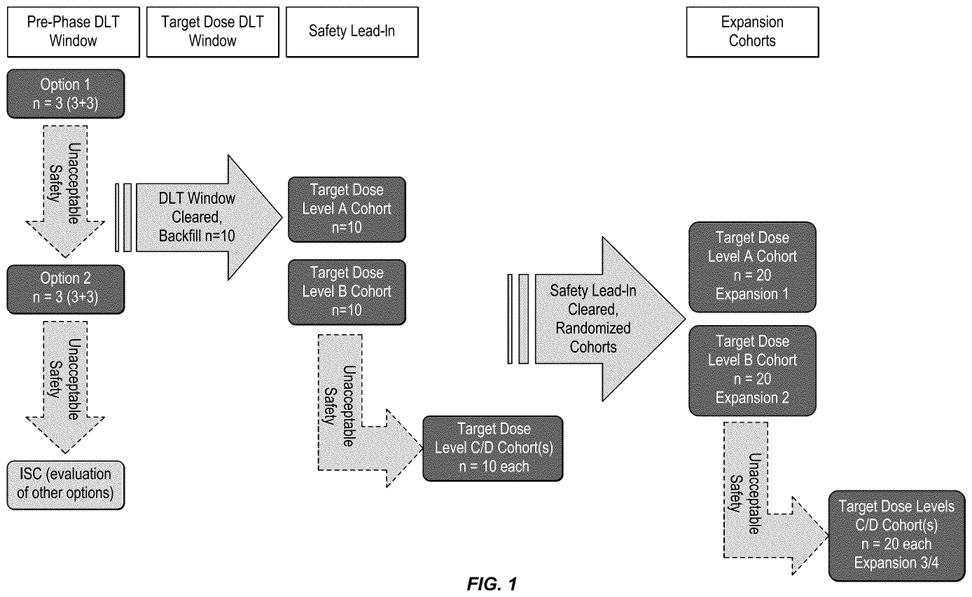

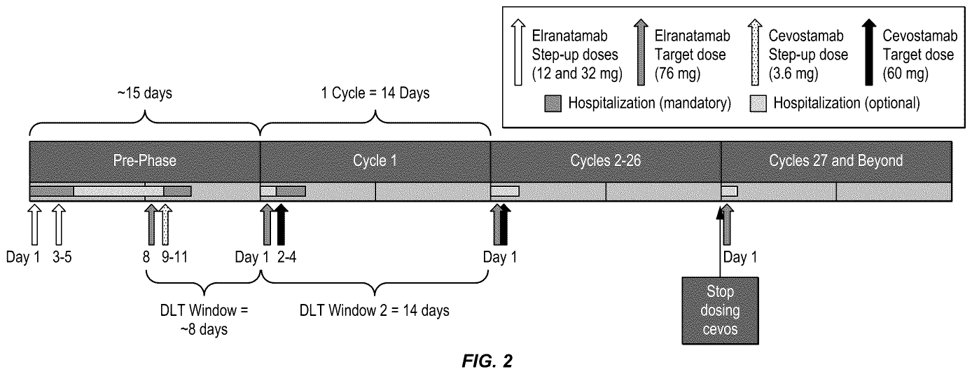

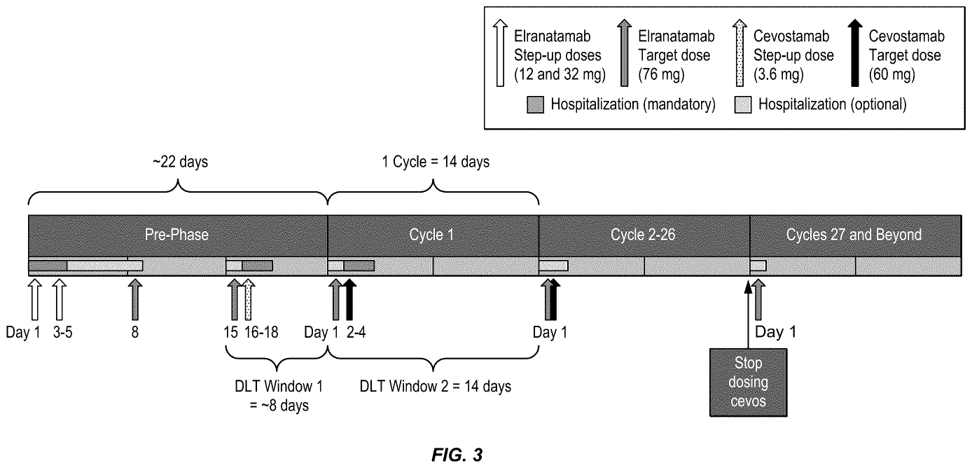

The overall study design is illustrated in FIG. 1. FIG. 2 provides further details of the Option 1 dosing schedule presented in FIG. 1. FIG. 3 provides further details of the Option 2 dosing schedule presented in FIG. 1.

### B. Dose-Limiting Toxicity (DLT) Assessment Stage

The initial dosing stage evaluates cevostamab in combination with elranatamab, with step-up dosing of each drug occurring over a pre-phase period of about 15 days. Following pre-phase, cevostamab and elranatamab target doses are administered in 14-day cycles.

### Pre-Phase Dosing

Study treatment is initially during pre-phase as follows (Option 1; see FIG. 2):

### Elranatamab

The initially proposed two step-up doses and target dose for elranatamab are 12 mg, 32 mg, and 76 mg, administered on pre-phase Day 1, Day 4 (i.e., Day 3, Day 4, or Day 5), and Day 8, respectively.
Patients undergo mandatory hospitalization for 48 hours after the first step-up dose and for 24 hours after the second step-up dose. Patients who experience symptoms of CRS with the prior dose may be considered for additional hospitalization at the discretion of the investigator.

### Cevostamab

The initial step-up dose for cevostamab is 3.6 mg, administered on pre-phase Day 9-11 (i.e., Day, 9, Day 10, or Day 11) (1-3 days following the first elranatamab target dose).
Patients undergo mandatory hospitalization for 48 hours after the step-up dose and first cevostamab target dose (administered after pre-phase on Day 2-4 (i.e., Day 2, Day 3, or Day 4) of Cycle 1). Patients who experience symptoms of CRS may be considered for additional hospitalization at the discretion of the investigator.

For this dosing schedule (Option 1), the first dose-limiting toxicity (DLT) assessment window (DLT Window 1) is defined as the period between the first elranatamab target dose (pre-phase Day 8) and the Cycle 1 Day 1 elranatamab target dose (about 8 days). Patients who discontinue study treatment prior to completion of the DLT assessment window for reasons other than a DLT are replaced.

### Pre-Phase Migration

If the CRS events in the pre-phase period exceed the safety threshold, the step-up staggering between cevostamab and elranatamab may be extended by 1 week to a pre-phase of about 22 days (Option 2; see FIG. 3). If this occurs, the treatment regimen will be the same as described above with the following changes:
Elranatamab is administered at the target dose on pre-phase Day 15 in addition to Day 8.
The initial step-up dose of cevostamab is administered during the pre-phase period on Day 16-18 (i.e., Day 16, Day 17, or Day 18) (1-3 days following the second elranatamab target dose).

For this dosing schedule (Option 2), DLT Window 1 is defined as the period between the second elranatamab target dose (pre-phase Day 15) and the Cycle 1 Day 1 elranatamab target dose (about 8 days).

### Cevostamab Without Step-Up Dose

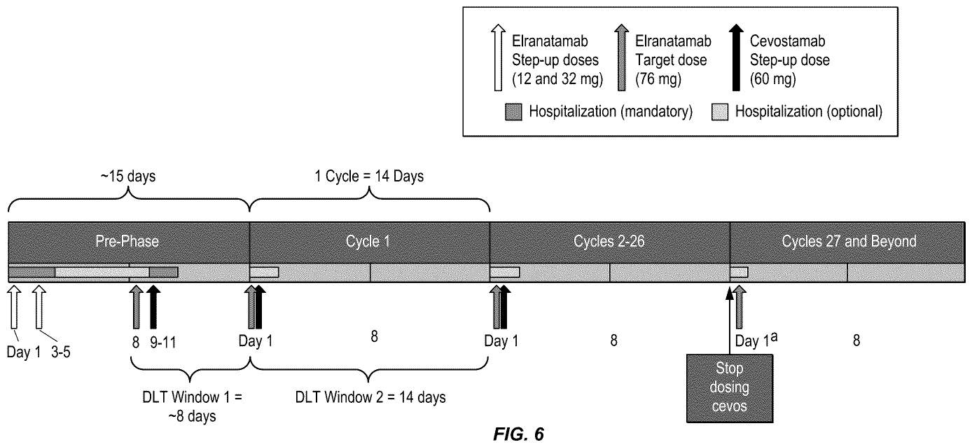

If Option 1 is tolerable and Option 2 mitigation is not necessary, then Option 3 may be tested in a separate cohort of approximately 10 patients in the expansion phase, to determine whether the cevostamab step-up dose may be eliminated (Option 3; see FIG. 6). In this cohort, the same DLT windows as for Option 1 would apply.

### Elranatamab

The initially proposed two step-up doses and target dose for elranatamab are 12 mg, 32 mg, and 76 mg, administered on pre-phase Day 1, Day 4, and Day 8, respectively.
Patients undergo mandatory hospitalization for 48 hours after the first step-up dose and for 24 hours after the second step-up dose. Patients who experience symptoms of CRS with the prior dose may be considered for additional hospitalization at the discretion of the investigator and Medical Monitor. Prolonged/optional hospitalization for observation only will not be considered a serious adverse event.

### Cevostamab

There will be no step-up dose of 3.6 mg.
The first dose of cevostamab will be at the target dose level e.g., 60 or 105 mg Q2W during Days 9-11 of pre-phase dosing. Patients will undergo mandatory hospitalization for 48 hours after the first cevostamab dose (i.e., target dose).
The second target dose of cevostamab will be given on Cycle 1 Day 1 at least 1 hour after the end of elranatamab administration and upon resolution of any infusion-related events including CRS (see Section 4.3.2.3). Hospitalization will be optional for Cycle 1 Day 1.

### Definition of Dose-Limiting Toxicity

Dose-limiting toxicities (DLTs) are treated according to clinical practice and will be monitored through their resolution. All adverse events are considered related to study treatment unless such events are attributed by the investigator to another clearly identifiable cause (e.g., disease progression, concomitant medication, or preexisting medical condition). Decreases in B cells, lymphopenia, and/or leukopenia due to decreases in B cells or T cells are not considered DLTs as they are expected pharmacodynamic outcomes of cevostamab and elranatamab treatment.

A DLT is defined as any of the following adverse events occurring during the DLT assessment windows, unless clearly attributed to another identified etiology by the investigator (e.g., MM progression):
Any Grade 4 non-hematologic adverse event or Grade 5 adverse event.
Any Grade ≥3 hematologic adverse event, with the following exceptions that will not be considered a DLT:
   Grade ≥3 lymphopenia, which is an expected outcome of therapy.
   Grade ≥3 neutropenia (ANC<500 cells/μL) lasting ≤7 days.
   Grade ≥3 febrile neutropenia lasting ≤3 days.
   Grade ≥3 thrombocytopenia lasting ≤7 days and not associated with clinically significant bleeding. Patients may receive platelet transfusions for MM-related thrombocytopenia.
   Grade 3 anemia.
Any Grade 3 non-hematologic, non-hepatic adverse event, with the following exceptions that will not be considered a DLT:
   Grade 3 anorexia, nausea, vomiting, constipation, or diarrhea that improves to Grade ≤2 with standard-of-care therapy in ≤3 days.
   Grade 3 fatigue lasting ≤3 days.
   Grade 3 tumor lysis syndrome (TLS), if it is successfully managed clinically and resolves ≤7 days without end-organ damage.
   Grade 3 infection that resolves to Grade ≤2 within 7 days and does not require intensive care unit management.
   Grade 3 fever (as defined by >40° C.) for ≤48 hours.
   Grade 3 adverse event of tumor flare (defined as local pain, irritation, or rash localized at sites of known or suspected plasmacytoma) that resolves to Grade ≤2 within 7 days.
   Grade 3 arthralgia that can be adequately managed with supportive care or that resolves to Grade ≤2 within 7 days.
   Grade 3 autoimmune thyroiditis or other endocrine abnormality that can be managed by endocrine therapy that would not necessitate initiation of systemic corticosteroids (with the exception of replacement corticosteroids for adrenal insufficiency).
   Fracture of any grade at sites of lytic bone disease.
   Grade 3 infusion-related reactions (IRRs). Generally, IRRs are not considered to be dose-related events on the basis of experience with monoclonal antibodies.
   Grade 3 laboratory abnormalities that are asymptomatic and are considered not to be clinically significant by the investigator.
Any hepatic function abnormality as defined by the following:
   Grade 3 elevation of serum bilirubin (total).
   Grade 3 elevation of serum hepatic transaminase (AST or ALT) lasting >7 days.
   AST or ALT>3× the upper limit of normal (ULN) and total bilirubin >2×ULN, with the following exception that will not be considered a DLT:
      Any AST or ALT>3× the ULN and total bilirubin >2× the ULN where no individual laboratory value exceeds Grade 3 that occurs in the context of Grade ≤2 CRS (as defined by the criteria established by Lee et al., Biol Blood Marrow Transplant, 25: 625-638, 2019); and all resolve to Grade ≤1 within <3 days.
Any CTCAE Grade 3 neurologic toxicity mapping to a MedDRA High-Level Group Term, High-Level Term, or Preferred Term selected from deliria (including confusion), seizures (including subtypes), coma states, cortical dysfunction, disturbances in consciousness not elsewhere classified (NEC), encephalopathies NEC, increased intracranial pressure disorders, memory loss (excluding dementia), mental disorders NEC, mental impairment (excluding dementia and memory loss), speech and language abnormalities, agitation, hypertonia, hyporesponsive to stimuli, slow response to stimuli, or unresponsive to stimuli that does not resolve to baseline within 2 days, or any Grade 2 neurologic toxicity mapping to a MedDRA High-Level Group Term, High-Level Term, or Preferred Term that does not resolve to baseline within 4 days.
Grade 1 depressed level of consciousness or Grade 1 dysarthria that does not resolve to baseline within 72 hours.
Any grade seizure.

### Clearance of the DLT Windows

Patients who discontinue from the study prior to completing the DLT assessment windows for reasons other than a DLT are considered non-evaluable for dose decisions and maximum tolerated dose (MTD) assessments, and are replaced by an additional patient at that same dose level. Patients who miss any dose during the DLT assessment windows for reasons other than a DLT are also replaced. Patients who receive supportive care (including radiotherapy) during the DLT assessment windows that confounds the evaluation of DLTs (not including supportive care described below as part of the DLT decision) may be replaced. Clearance of the DLT windows occurs in accordance with the rules listed below:
A minimum of 3 patients is initially enrolled in the DLT cohort.
If none of the first 3 DLT-evaluable patients experiences a DLT, backfill enrollment of 7 patients may proceed (total n=10).
If 1 of the first 3 DLT-evaluable patients experiences a DLT, the cohort is expanded to 6 patients. If there are no further DLTs in the first 6 DLT-evaluable patients, backfill enrollment of 4 patients may proceed (total n=10).
If ≥2 of the first 6 DLT-evaluable patients experience a DLT, the pre-phase mitigation plan is initiated (i.e., Option 2, delaying cevostamab step-up dosing by a week).
If, during the pre-phase mitigation plan (i.e., Option 2), >1 DLT is experienced in a cohort of 6 DLT-evaluable patients, additional mitigation plans are considered.

In the initial dosing cohort of 3 patients, treatment is staggered such that the second patient enrolled in the cohort receives the first dose of study treatment at least 72 hours after the first enrolled patient receives the first dose of study treatment in order to assess for any severe and unexpected acute drug or injection-related toxicities; dosing in subsequent patients in each cohort is staggered by at least 24 hours from the end of the prior patients' administration.

### C. Safety Lead-In

After the initial patients have cleared the DLT Window 1 for pre-phase step-up doses for both study treatments, patients continue onto the safety lead-in and receive the lowest range of target doses for cevostamab and elranatamab, referred to as the Target Dose Level A (see FIG. 1): cevostamab 60 mg IV once every two weeks (Q2W) and elranatamab 76 mg SC Q2W. For the safety lead-in, DLT Window 2, which includes the first cevostamab target dose, is defined as the period between the second elranatamab target dose (Cycle 1 Day 1) and the third elranatamab target dose (Cycle 1 Day 14). If the initial DLT cohort clears both DLT windows, then it is backfilled to 10 patients as part of the safety lead-in cohort.

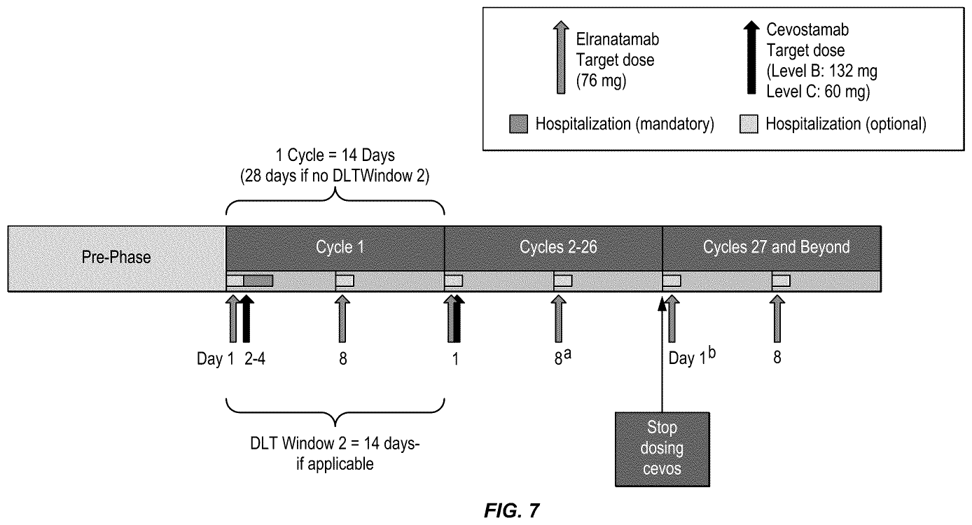

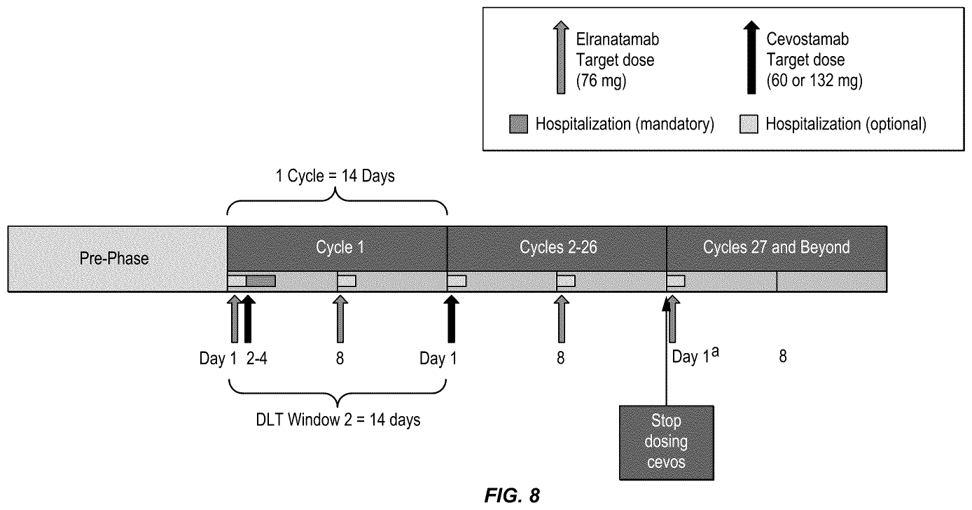

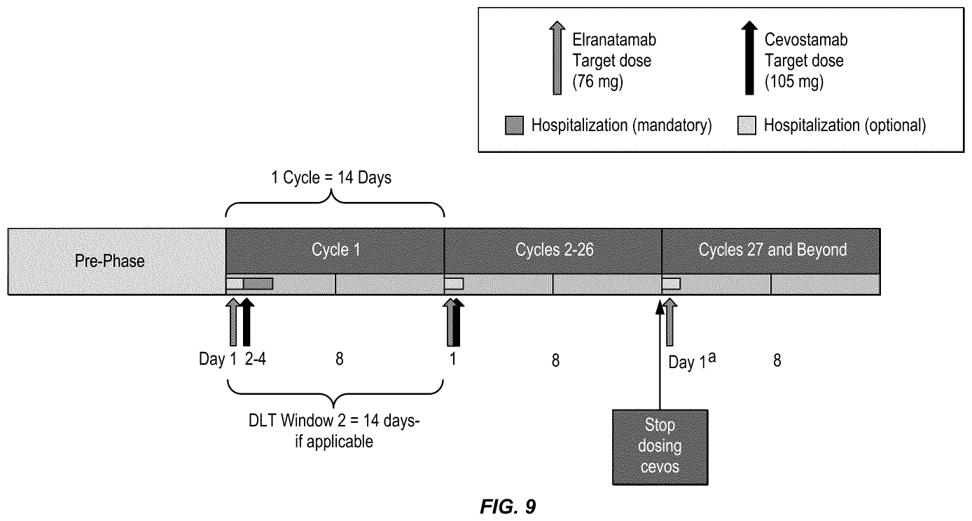

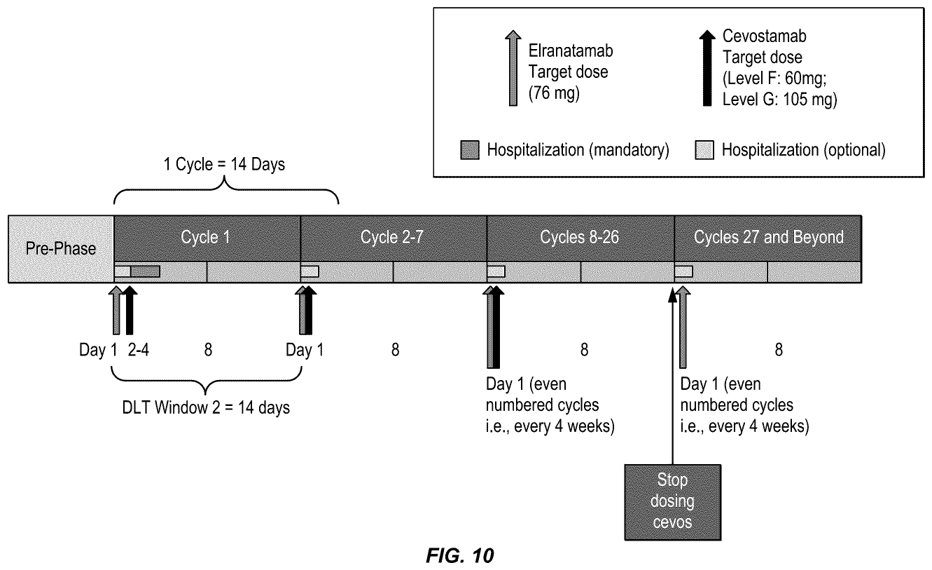

If the Target Dose Level A is deemed safe and tolerable after patients have had at least one cycle of treatment, testing of a subsequent target dose level will be initiated; see Table 6 and FIG. 7-10). Approximately 10 patients will be enrolled at the target dose level for this safety lead-in cohort. If a target dose level is not tolerated, an additional safety lead-in cohort at a lower dose level may be initiated (see Table 6 and FIG. 7-10). If a safety lead-in cohort is initiated at a target dose level higher than what was previously deemed safe and tolerable (e.g., E following A), then clearance of the DLT windows will occur as described above. The totality of data from Target Dose Level A and any subsequent cohorts will be evaluated by the internal safety committee (ISC) to determine the range of doses to take forward in the dose expansion.

Doses may be delayed to allow patients to recover from toxicity or for other selected reasons.

TABLE 6
Potential Target Dose Levels

| Target Dose Level | Cevostamab | Elranatamab |
|---|---|---|
| A (n = 10 ^{a}) | 60 mg Q2W | 76 mg Q2W |
| B (n = 10 ^{a}) | 132 mg Q2W | 76 mg Q2W |
| C (n = 10 ^{b}) | 60 mg Q2W | 76 mg Q2W |
| D (n = 10 ^{b}) | 60 or 132 mg Q2W | 76 mg Q2W |
| E (n = 10) | 105 mg Q2W | 76 mg Q2W |
| F (n = 10) | 60 mg Q2W | 76 mg Q2W |
| | for 8 cycles, | for 8 cycles, |
| | then Q4W | then Q4W |
| G (n = 10) | 105 mg Q2W | 76 mg Q2W |
| | for 8 cycles, | for 8 cycles, |
| | then Q4W | then Q4W |

Q2W = every two weeks; Q4W = every four weeks; QW = once a week.
^{a} Initial patients from DLT assessment stage plus backfill.
^{b} Mitigation for other dose levels; may not be implemented.

### D. Dose-Expansion Stage

The dose-expansion stage for this study is designed to further characterize the safety, tolerability, pharmacokinetics, and activity of the study treatment.

Based on pharmacokinetic (PK), pharmacodynamic (PD), safety, and activity analysis and modeling approaches, one or more expansion cohorts may be opened to, for example, further investigate an alternate study treatment dose or schedule. The dose(s) selected for cevostamab and elranatamab for expansion must have been cleared in the initial backfill cohort and safety lead-in. Not all expansion cohorts need to be opened at the same time. If more than one expansion cohort is open at the same time, then patients are assigned to a cohort via equal randomization in order to reduce potential selection bias.

Following review of the available safety data from the safety lead-in, the cevostamab and/or elranatamab mandatory hospitalization periods for the expansion cohorts may be adjusted. The cevostamab step-up dose (Option 3) may also be eliminated in a small cohort of patients (e.g., approximately 10).

Approximately 20 patients are enrolled into each of the expansion cohorts. Patients are treated at previously cleared doses (at or below the MTDs) and schedules.

All available safety data from the expansion cohort(s) are evaluated on an ongoing basis to assess the tolerability of the dose levels studied. At no time will a cevostamab and elranatamab dose level studied in the expansion stage exceed the highest dose level tested and cleared in the safety lead-in stage. Additionally, interim analyses are conducted to guide potential early stopping of enrollment in the event of unacceptable toxicity/tolerability.

### E. Cevostamab Re-Treatment

Investigators may re-treat patients who may benefit from additional cycles of cevostamab and who initially responded to study treatment, but subsequently developed recurrent or progressive disease either after the completion of cevostamab therapy or after a dose delay of more than 28 days. Patients are eligible for cevostamab re-treatment as described below.

The cevostamab dose and schedule for these patients is the dose and schedule that has been found to be safe at the time of re-treatment, provided the following criteria are met:
Pertinent eligibility criteria are met at the time that cevostamab treatment is re-initiated, with the following exceptions:
   Prior therapy with cevostamab is allowed.
   The investigator and patient would like to pursue cevostamab re-treatment, despite the appropriateness and availability of any new therapies for the treatment of MM.
   Manageable and reversible immune-related adverse events with initial cevostamab treatment are allowed and do not constitute an exclusionary history of autoimmune disease.
   Radiotherapy is allowed within 4 weeks of cevostamab re-treatment.
Patients must have had documented objective response (complete response (CR), very good partial response (VGPR), or partial response (PR)) per IMWG criteria at the end of initial cevostamab treatment and for at least one post-treatment tumor assessment after the end of treatment.
Patients without biochemical disease progression (defined as an increase of monoclonal paraprotein in absence of organ dysfunction and clinical symptoms) but who have clear indication of recurrent disease (i.e., development of new bone lesions or soft tissue plasmacytomas or an increase in size of existing bone lesions or soft tissue plasmacytomas) are allowed.
Patients must not have experienced Grade 4 non-hematologic adverse events related to cevostamab during initial cevostamab treatment.
Patients who experienced Grade 2 or Grade 3 adverse events during initial treatment must have resolved these toxicities to Grade ≤1.
With the exception of elranatamab, no intervening systemic anti-cancer therapy was administered between the completion of initial cevostamab treatment and re-initiation of cevostamab treatment.

Patients who have received fewer than 26 cycles of treatment but meet all re-treatment criteria may be qualified for re-treatment.

The dose and schedule of cevostamab to be administered for patients receiving re-treatment is on a previously tested dose and schedule that has cleared the DLT observation period.

The above rules for re-treatment apply both to the safety lead-in and expansion cohorts.

### F. End of Study Definition

The end of this study is defined as the date when the last patient has completed his or her last visit or the date at which the last data point required for statistical analysis or safety follow-up (90 days following the final dose) is received from the last patient, whichever occurs later. The end of the study is expected to occur approximately 36 months after the last patient is enrolled.

### G. Patients

Approximately 60-120 patients with relapsed/refractory multiple myeloma (R/R MM) are enrolled in the study.

### H. Inclusion Criteria

Patients must meet the following criteria for study entry:
Age ≥18 years.
Eastern Cooperative Oncology Group (ECOG) Performance Status of 0 or 1.
Life expectancy of at least 12 weeks.
Diagnosis of relapsed/refractory multiple myeloma (R/R MM) per International Myeloma Working Group (IMWG) criteria
Resolution of adverse events from prior anti-cancer therapy to Grade ≤1, with the following exception: any grade alopecia is allowed.
Measurable disease, defined as at least one of the following:
   Serum M-protein ≥0.5 g/dL (≥5 g/L).
   Urine M-protein ≥200 mg/24 hours.
   Serum free light chain (SFLC) assay: Involved SFLCs≥10 mg/dL (≥100 mg/L) and an abnormal SFLC ratio (<0.26 or >1.65).
Laboratory values as follows (requirement prior to first dose of study treatment):
   Hepatic function:
   AST and ALT≤2.5×upper limit of normal (ULN).
   Total bilirubin ≤1.5×ULN; patients with a documented history of Gilbert syndrome and in whom total bilirubin elevations are accompanied by elevated indirect bilirubin are eligible if total bilirubin ≤3×ULN.
Hematologic function:
Platelet count ≥75,000/mm3.
ANC≥1000/mm3.
Total hemoglobin ≥8 g/dL.

Note: Patients should not receive supportive care (e.g., transfusion, granulocyte colony-stimulating factor (G-CSF), etc.) within 7 days prior to the first treatment to meet hematologic function eligibility criteria.
Creatinine clearance (CrCl)≥30 mL/min (either calculated using modified Cockcroft-Gault equation or per 24-hr urine collection).
Serum calcium (corrected for albumin) level ≤11.5 mg/dL (treatment of hypercalcemia is allowed and patient may enroll if hypercalcemia returns to Grade ≤1 with standard treatment).
For women of childbearing potential: agreement to remain abstinent (refrain from heterosexual intercourse) or use contraception.
For men: agreement to remain abstinent (refrain from heterosexual intercourse) or use a condom, and agreement to refrain from donating sperm.

### I. Exclusion Criteria

Patients who meet any of the following criteria are excluded from study entry:
Pregnancy or breastfeeding, or intention of becoming pregnant during the study or within 5 months after the final dose of study treatment.
Prior treatment with cevostamab or another agent targeting FcRH5.
Prior treatment with elranatamab.
Prior treatment with CD3-engaging therapies or antibody-drug conjugate (ADC) treatment without the specified washout period before the first dose of study treatment (washout period of 6 months for B-cell maturation antigen (BCMA) CD3-engaging therapies or BCMA-targeted ADC treatment; washout period of 3 months for non-BCMA-targeted CD3-engaging therapies).
Prior treatment with allogenic or autologous chimeric antigen receptor (CAR) T-cell therapy without the specified washout period before the first dose of study treatment (washout of 6 months for BCMA CAR-T therapy; washout of 3 months for non-BCMA CAR-T therapy).
Prior use of any monoclonal antibody, radioimmunoconjugate, or non-BCMA-targeted ADC as anti-cancer therapy within 4 weeks before first dose of study treatment, except for the use of non myeloma therapy (e.g., denosumab for hypercalcemia is allowed).
Treatment with any chemotherapeutic agent, or treatment with any other anti-cancer agent (investigational or otherwise) within 4 weeks or 5 half-lives of the drug, whichever is shorter, prior to first dose of study treatment.
Known treatment-related, immune-mediated adverse events associated with prior immunotherapeutic agents, as follows:
   Prior PD-L1/PD-1 or CTLA-4 inhibitor: Grade □ 3 adverse events with the exception of Grade 3 endocrinopathy managed with replacement therapy.
   Grade 1-2 adverse events that did not resolve to baseline after treatment discontinuation.
Treatment with radiotherapy within 4 weeks (systemic radiation) or 14 days (focal radiation) prior to first study treatment.
Inability to comply with protocol-mandated hospitalization and activities restrictions.
Autologous SCT within 100 days prior to first dose of study treatment.
Prior allogeneic SCT.
Absolute plasma cell count exceeding 500/mL or 5% of the peripheral blood white cells.
Diagnosis of Waldenstrom macroglobulinemia or POEMS (polyneuropathy, organomegaly, endocrinopathy, myeloma protein, and skin changes) syndrome.
Patients with known history of amyloidosis (e.g., positive Congo Red stain or equivalent in tissue biopsy).
Prior solid organ transplantation.
History of autoimmune disease, including, but not limited to, Guillain-Barre syndrome (GBS, or variants), myasthenia gravis, myositis, autoimmune hepatitis, systemic lupus erythematosus, rheumatoid arthritis, inflammatory bowel disease, vascular thrombosis associated with antiphospholipid syndrome, Wegener's granulomatosis, Sjögren's syndrome, multiple sclerosis, vasculitis, or glomerulonephritis. Patients with a history of autoimmune-related hypothyroidism on a stable dose of thyroid replacement hormone may be eligible for this study.
History of confirmed progressive multifocal leukoencephalopathy.
History of Grade ≥3 peripheral motor polyneuropathy.
History of severe allergic or anaphylactic reactions to monoclonal antibody therapy (or recombinant antibody-related fusion proteins).
History of other malignancy within 2 years prior to screening, except those with negligible risk of metastasis, relapse, or death, such as ductal carcinoma in situ not requiring chemotherapy, appropriately treated carcinoma in situ of the cervix, non-melanoma skin carcinoma, low-grade, localized prostate cancer (Gleason score □ 7) not requiring treatment, or appropriately treated Stage I uterine cancer.
Current or past history of CNS disease, such as stroke, epilepsy, CNS vasculitis, neurodegenerative disease, or CNS involvement by MM. Patients with a history of stroke who have not experienced a stroke or transient ischemic attack in the past 2 years and have no residual neurologic deficits as judged by the investigator are allowed. Patients with a history of epilepsy who have had no seizures in the past 2 years while not receiving any anti-epileptic medications are allowed.
Significant cardiovascular disease (such as, but not limited to, New York Heart Association Class III or IV cardiac disease, myocardial infarction within the last 6 months, uncontrolled arrhythmias, or unstable angina) that may limit a patient's ability to adequately respond to a CRS event.
Symptomatic active pulmonary disease requiring supplemental oxygen.
Prolonged QT syndrome (or QTcF □ 470 msec at screening).
Recent major surgery within 4 weeks prior to first dose of study treatment. Protocol-mandated procedures (e.g., bone marrow biopsies) are permitted.
Known active bacterial, viral, fungal, mycobacterial, parasitic, or other infection (excluding fungal infections of nail beds) receiving treatment within 28 days of first dose.
Known or suspected chronic active CMV and/or EBV infection with positive and quantifiable EBV PCR or CMV PCR prior to first study treatment.
Known history of Grade ≥3 CRS or immune effector cell-associated neurotoxicity syndrome with prior bispecific therapies.
Known history of hemophagocytic lymphohistiocytosis or macrophage activation syndrome.
Positive serologic or PCR test results for acute or chronic HBV infection. Patients whose HBV infection status cannot be determined by serologic test results must be negative for HBV by PCR to be eligible for study participation.
Acute or chronic HCV infection. Patients who are positive for HCV antibody must be negative for HCV by PCR to be eligible for study participation.
Known history of HIV seropositivity.
Administration of a live, attenuated vaccine within 4 weeks before first dose of study treatment or anticipation that such a live attenuated vaccine will be required during the study.
Treatment with systemic immunosuppressive medications (including, but not limited to, cyclophosphamide, azathioprine, methotrexate, thalidomide, and anti-tumor necrosis factor agents) with the exception of corticosteroid treatment ≤10 mg/day prednisone or equivalent within 2 weeks prior to first dose of study treatment. The use of inhaled corticosteroids is permitted. The use of mineralocorticoids for management of orthostatic hypotension is permitted. The use of physiologic doses of corticosteroids for management of adrenal insufficiency is permitted.
Any medical condition or abnormality in clinical laboratory tests that, in the investigator's judgment, precludes the patient's safe participation in and completion of the study, or which could affect compliance with the protocol or interpretation of results.

### J. Method of Treatment Assignment

GO43979 is an open-label study. The Safety Lead-in cohorts are non-randomized, while the expansion cohorts are randomized with equal allocation via block randomization. Should an expansion cohort complete enrollment, be dropped for safety reasons, or be added for investigation, randomization in the remaining cohorts is re-adjusted to continue, again with equal allocation via block randomization. The purpose of randomizing expansion cohorts is to reduce potential selection bias between them.

### K. Study Treatment Dosage, Administration, and Compliance

Treatment regimens are summarized in Example 1, above.

### Hospitalization

Hospitalization requirements are summarized in Table 7, below.

TABLE 7
Hospitalization Requirements

| Pre-Phase | Pre-Phase | Cycle 1 Day 1 | Cycle 1 Day 2 | Cycle 2 Day 1, |
|---|---|---|---|---|
| Elranatamab | Cevostamab | Elranatamab | Cevostamab | etc. |
| Hospitalization | Hospitalization | Hospitalization | Hospitalization | Hospitalization |
| required 48 hours | required 48 | optional. | required for at | optional. |
| after 1^{st} step-up | hours after | | least 48 hours | |
| dose and 24 hours | step-up dose. | | after the 1^{st} target | |
| after 2^{nd} step-up | | | dose. | |
| dose. | | | | |
| Hospitalization | | | | |
| optional after 1st | | | | |
| target dose. | | | | |

Administration of cevostamab and elranatamab is performed in a clinical setting with immediate access to a critical care unit and staff who are trained to monitor for and respond to medical emergencies. Neurology consultation services should be readily available to address any neurologic adverse events that may arise as a result of study treatment.

### Pre-Phase

Patients are hospitalized for at least 48 hours after the first step-up dose of elranatamab (Day 1) and 24 hours after second step-up dose of elranatamab (Day 3-5). These two step-up doses of elranatamab are likely to be administered during a single hospitalization.

Patients must be hospitalized for at least 48 hours after completion of the step-up cevostamab (Day 9-11) infusion administered during pre-phase. Prolonged/optional hospitalizations for observation only are not considered serious adverse events.

### Cycle 1

Patients are hospitalized for at least 48 hours after completion of the initial target dose of cevostamab (Cycle 1, Day 2).

### Cycle 2 and Beyond

For Cycle 2 and beyond, patients may receive elranatamab and cevostamab doses in the outpatient clinical setting. However, the patient may be observed in a hospital inpatient unit after study treatment administration if warranted. Prolonged/optional hospitalizations for observation only are not considered serious adverse events.

### Hospital Discharge Guidance

Prior to discharge (e.g., 24 or 48 hours after completion of infusion, as applicable), no evidence of ongoing cytokine release syndrome/infusion-related reaction (CRS/IRR), macrophage activation syndrome (MAS)/hemophagocytic lymphohistiocytosis (HLH), or neurologic toxicity should be detected on physical examination or with relevant laboratory results. In addition, all vital signs and 02 saturation must have returned to baseline (predose), and abnormal laboratory values must not necessitate hospitalization or additional monitoring. Otherwise, the patient should remain in inpatient hospitalization until CRS/IRR, MAS/HLH, neurologic toxicity, vital signs, or abnormal laboratory values resolve or return to baseline.

### Additional Hospitalization Requirements

Hospitalization requirements beyond those described above are determined on the basis of the clinical course during the most recent study drug infusions. Doses administered on an outpatient basis follow observation times as provided below. Following study treatment administration and subsequent observation, patients must indicate no evidence of ongoing CRS/IRR, MAS/HLH, or neurologic toxicity prior to discharge.

### L. Premedication

Premedication is administered prior to study treatment on each dosing day as described in Table 8, below.

TABLE 8
Premedication Regimen

| | Dose and | |
|---|---|---|
| Premedication | Route | Timepoints |
| Dexamethasone ^{a} | 20 mg IV | Pre-Phase and Cycle 1: 1 hour (±15 |
| | | minutes) prior to study treatment. |
| | | Cycles 2 and Beyond: 1 hour (±15 |
| | | minutes) prior to study treatment if |
| | | patient experienced CRS with the |
| | | prior administration |
| Acetaminophen/ | 500-1000 mg | Prior to study treatment (all cycles) |
| paracetamol | per os (PO) | |
| Diphenhydramine ^{b} | 25-50 mg | Prior to study treatment (all cycles) |
| | PO | |

Note:
If multiple treatments are administered within 24 hours, premedications are given only once prior to the first treatment administered.
^{a} Dexamethasone 20 mg IV preferred; alternative corticosteroid equivalent such as methylprednisolone 80 mg IV is also acceptable.
^{b} For sites that do not have access to diphenhydramine, an equivalent medication may be substituted per local practice.

### M. Cevostamab

Cevostamab is administered to patients by IV infusion to well-hydrated patients, using standard medical syringes and syringe pumps or IV bags where applicable. Cevostamab is administered on a single-step-up schedule with the step-up dose (3.6 mg) administered on Day 9-11 of pre-phase and the first target dose administered ˜7 days after the step-up dose. Target doses are administered every 2 weeks: on Day 2 in Cycle 1 and on Day 1 during Cycles 2-26.

Initially, cevostamab is administered over 4 hours (±15 minutes). The infusion may be slowed or interrupted for patients experiencing IRRs. If repeat dosing of the initial step-up dose is required due to a dose delay, the next two doses (one step-up dose and first target dose) should be administered over 4 hours. In the absence of IRRs and CRS after receiving the first target dose, the infusion time of cevostamab in subsequent cycles may be reduced to 2 hours.

During pre-phase and Cycle 1, there must be a minimum of 24 hours from the end of subcutaneous administration of the first elranatamab target dose to the initiation of the cevostamab step-up dose and resolution of any infusion-related events including CRS.

For Cycle 2 and beyond, cevostamab may be infused after at least 1 hour after the end of elranatamab administration and upon resolution of any infusion-related events including CRS. Patients who do not require hospitalization will be observed at least 90 minutes for fever, chills, rigors, hypotension, nausea, or other signs and symptoms of IRRs following each subsequent cevostamab infusion.

### N. Elranatamab

Elranatamab is administered subcutaneously to well-hydrated patients over about 2 minutes, preferably into the subcutaneous tissue of the lower abdomen.

A minimum of 2 days is maintained between the two step-up doses (pre-phase D1 and D4), and a minimum of3 days between the pre-phase D4 dose and the first target dose (D8); a minimum of 6 days is maintained between doses thereafter.

### O. Tocilizumab

Tocilizumab is administered when indicated for treatment-emergent cytokine release syndrome (CRS), as described below in Table 9.

TABLE 9
Recommendations for Management of Cytokine Release Syndrome
Severe COVID-19 is associated with a CRS involving the inflammatory cytokines IL-6,
IL-10, IL-2, and interferon-gamma (IFN--y). Cevostamab and elranatamab have a potential
risk of CRS; therefore, if a trial patient develops severe CRS during study course, a
differential diagnosis included testing to rule out COVID-19 infection should be considered.

| | | Actions with Study Drug | |
|---|---|---|---|
| Signs/Symptoms | ASTCT | and Additional | Future Actions with |
| of CRS ^{a} | Grade | Management ^{a} | Study Drug ^{a} |
| Clinical | 1 | Immediate actions: | Next dose: |
| Presentation A: | | Hold further study treatment | Pretreat as described in |
| Temperature ≥38° C. | | infusion until symptoms are | Section L. |
| (100.4° F.) b | | completely resolved. | Pretreat with IV |
| with no hypotension | | Restarting infusion: | corticosteroids as |
| or hypoxia | | If study treatment is | described Section L. |
| | | administered by infusion, wait | As clinically indicated, |
| | | until 30 minutes after the | and if study treatment is |
| | | event has completely resolved | administered by infusion, |
| | | before restarting the infusion | extend infusion time |
| | | at up to 50% of the original | (slower infusion rate) for |
| | | infusion rate. | next dose. ^{e} |
| | | Additional Management: | As clinically indicated, |
| | | Provide supportive care, | hospitalize patient for next |
| | | manage fever, constitutional | dose. |
| | | symptoms and organ toxicities | |
| | | as required. ^{c} | |
| | | Monitor patient until | |
| | | symptoms | |
| | | resolve; hospitalize patient as | |
| | | clinically indicated. | |
| | | In patients with clinically | |
| | | significant CRS | |
| | | signs/symptoms | |
| | | and/or comorbidities (e.g., | |
| | | impaired cardiovascular | |
| | | function, reduced pulmonary | |
| | | reserve) or prolonged fever | |
| | | (>4-6 hours) administer IV | |
| | | corticosteroids (for example, | |
| | | dexamethasone 10 mg IV | |
| | | every 6 hours until resolution. | |
| | | Note, if neurologic symptoms | |
| | | are | |
| | | present then dexamethasone is | |
| | | the preferred corticosteroid). | |
| | | If patient's signs/symptoms of | |
| | | CRS remain refractory 24 hours | |
| | | after the first corticosteroid | |
| | | dose, | |
| | | then tocilizumab 8 mg/kg IV | |
| | | can be administered. ^{d} | |
| Clinical | 2 | Immediate actions: | Next dose: |
| Presentation B: | | If study treatment is | If CRS signs/symptoms |
| Temperature ≥38° C. | | administered by infusion, stop | resolve completely for at |
| (100.4° F.) b | | study treatment infusion. | least 24 hours before the |
| With | | Restarting infusion: | next planned dose, then |
| Mild | | If study treatment is | study drug can be |
| hypotension | | administered by infusion, do | administered as follows: |
| and/or | | not restart study treatment | Pretreat as described in |
| Mild hypoxia that | | infusion. | Section L. |
| requires | | Additional Management: | Pretreat with IV |
| intervention | | Provide supportive care, | corticosteroids as |
| | | manage fever, constitutional | described in Section L. |
| | | symptoms and organ toxicities | If study treatment is |
| | | as required. ^{c} | administered by infusion, |
| | | Hospitalize patient until | administer study drug |
| | | complete resolution of signs | infusion at up to 50% of |
| | | and symptoms. | the initial infusion rate of |
| | | Monitor cardiac and other | the previous dose if the |
| | | organ function closely. | event occurred during or |
| | | Monitor fluid balance; | within 24 hours of the |
| | | administer IV fluid boluses | infusion. ^{e} |
| | | 250-500 mL up to 2000 mL | If clinically indicated, |
| | | total, while avoiding fluid | hospitalize patient for at |
| | | overload. | least 24 hours after the |
| | | Provide oxygen for hypoxia, | injection/infusion. |
| | | e.g., low flow nasal cannula | Subsequent doses: |
| | | (≤6 | As clinically indicated |
| | | L/minute) or blow-by. | and if study treatment is |
| | | Administer IV | administered by infusion, |
| | | corticosteroids (for example, | extend infusion time |
| | | dexamethasone 10 mg IV | (slower infusion rate) for |
| | | every 6 hours until resolution. | subsequent doses. |
| | | Note, if neurologic symptoms | Should there be an |
| | | are present then | occurrence of CRS with a |
| | | dexamethasone is the | subsequent dose, manage |
| | | preferred corticosteroid). | as per the guidance in |
| | | If patient is not responding as | this table, based on the |
| | | expected after the addition of | patient's clinical |
| | | corticosteroids, administer | presentation. Additionally, |
| | | tocilizumab 8 mg/kg IV. ^{d} | for patients who have |
| | | For ongoing clinical | hemodynamic instability, |
| | | deterioration, | and/or worsening hypoxia |
| | | manage as per guidance for | or respiratory distress (i.e., |
| | | patients with Clinical | Clinical Presentations C or |
| | | Presentation C: | D), permanently |
| | | Initiate workup and assess | discontinue study |
| | | for signs and symptoms of | drug regardless of |
| | | HLH. | recovery. |
| Clinical | 3 | Immediate actions: | Next dose: |
| Presentation C: | | If study treatment is | If the patient has had |
| Temperature ≥38° C. | | administered by infusion, stop | two |
| (100.4° F.) b | | study treatment infusion. | presentations of CRS that |
| with | | Restarting infusion: | requires vasopressor |
| Hemodynamic | | If study treatment is | support and/or high-flow |
| instability despite | | administered by infusion, do | oxygen, permanently |
| Intravenous fluid | | not restart study treatment | discontinue study |
| and/or | | infusion. | drug. |
| Worsening | | Additional Management: | If CRS signs/symptoms |
| hypoxia or | | Provide supportive care, | resolve completely for at |
| respiratory | | manage fever, constitutional | least 72 hours before the |
| distress despite | | symptoms and organ toxicities | next planned dose, then |
| low- flow oxygen | | as required. ^{c} | study drug can be |
| administration | | Monitor cardiopulmonary and | administered as follows: |
| | | organ function in ICU. | Notify Medical Monitor. |
| | | Provide vasopressor support, | Pretreat as described in |
| | | with or without vasopressin, | Section L. |
| | | for hypotension as required. | Pretreat with IV |
| | | Provide oxygen for hypoxia, | corticosteroids as |
| | | e.g., high-flow nasal cannula | described in Section L. |
| | | (>6 L/minute), face mask, | If study treatment is |
| | | non-rebreather mask, or | administered by infusion, |
| | | Venturi mask. | administer study drug |
| | | Administer tocilizumab 8 | infusion at up to 50% of |
| | | mg/kg IV d and IV | the initial infusion rate of |
| | | corticosteroids (for example, | the previous dose if the |
| | | dexamethasone 10 mg IV | event occurred during or |
| | | every 6 hours until resolution. | within 24 hours of the |
| | | Note, if neurologic symptoms | infusion. ^{e} |
| | | are present then | Hospitalize patient for at |
| | | dexamethasone is the | least 24 hours after the |
| | | preferred corticosteroid). | injection/infusion. |
| | | For ongoing clinical | If this CRS event |
| | | deterioration, manage as per | occurred following the |
| | | guidance provided in Clinical | pre-phase Day 1 step-up |
| | | Presentation D. | dose, then the step-up |
| | | Initiate workup and assess | dose must be repeated. |
| | | for signs and symptoms of HLH | Subsequent doses: |
| | | as described in Example 3. | If study treatment is |
| | | Notify Medical Monitor. | administered by |
| | | | infusion, extend |
| | | | infusion time (slower |
| | | | infusion rate) for |
| | | | subsequent doses as |
| | | | clinically indicated. |
| | | | Should there be an |
| | | | occurrence CRS with a |
| | | | subsequent dose, |
| | | | manage as per the |
| | | | guidance this table, |
| | | | based on the patient's |
| | | | clinical presentation. |
| | | | Additionally, for |
| | | | patients who have |
| | | | hemodynamic |
| | | | instability, and/or |
| | | | worsening hypoxia or |
| | | | respiratory distress (i.e., |
| | | | Clinical Presentations C |
| | | | or D), permanently |
| | | | discontinue study drug |
| | | | regardless of recovery. |
| Clinical | 4 | Immediate actions: | Next and subsequent |
| Presentation D: | | If study treatment is | doses: |
| Temperature ≥38° C. | | administered by infusion, stop | Permanently |
| (100.4° F.) b | | study treatment infusion. | discontinue study |
| with | | Restarting infusion: | treatment |
| Hemodynamic | | If study treatment is | |
| Instability despite | | administered by infusion, do | |
| Intravenous fluid | | not restart study treatment | |
| and one vasopressor | | infusion. | |
| (excluding | | Additional Management: | |
| vasopressin) | | Provide supportive care, | |
| and/or | | manage fever, constitutional | |
| Worsening | | symptoms and organ toxicities | |
| hypoxia or | | as required. ^{c} | |
| respiratory distress | | Patient requires ICU | |
| despite high- flow | | admission for hemodynamic | |
| oxygen | | monitoring, and/or mechanical | |
| administration | | ventilation, and/or IV fluids | |
| | | and | |
| | | vasopressors as needed. | |
| | | Provide vasopressor support | |
| | | for hypotension with multiple | |
| | | vasopressors as required. | |
| | | Provide positive pressure | |
| | | oxygen for hypoxia. | |
| | | Administer tocilizumab 8 | |
| | | mg/kgIV d and IV | |
| | | corticosteroids (for example, | |
| | | dexamethasone 10 mg IV | |
| | | every 6 hours until resolution. | |
| | | Note, if neurologic symptoms | |
| | | are present then | |
| | | dexamethasone is the | |
| | | preferred corticosteroid). | |
| | | For patients who are | |
| | | refractory to tocilizumab | |
| | | therapy, another anti- | |
| | | cytokine agent can be | |
| | | administered. | |
| | | Initiate workup and | |
| | | assess for signs and | |
| | | symptoms of HLH as | |
| | | described in Example 3. | |

CRS = cytokine release syndrome; HLH = hemophagocytic lymphohistiocytosis; ICU = intensive care unit; IV = intravenous.
Note:
CRS is characterized by a supraphysiologic response following any immune therapy that results in the activation or engagement of endogenous or infused T cells and/or other immune effector cells. Symptoms can be progressive, must include fever at the onset, and may include hypotension, capillary leak (hypoxia) and end-organ dysfunction (Lee et al. 2019).
^{a} Guidance for CRS management based on Lee et al. (2019) and Riegler et al. Ther Clinical Risk Manag, 15: 323-335 (2019).
b Fever may not be prominent in patients who have received antipyretics, corticosteroids, or anticytokine therapy. In this case, management is driven by hypotension and/or hypoxia.
^{c} Patients should be treated with acetaminophen/paracetamol and an antihistamine (e.g., diphenhydramine) if they have not been administered in the previous 4 hours. For bronchospasm, urticaria, or dyspnea, treat as clinically indicated. Perform an infection workup, including assessment for bacterial, fungal, and viral infections, and cultures. Treat fever and neutropenia as required; administer broad-spectrum antibiotics and/or granulocyte colony-stimulating factor if indicated.
^{d} Tocilizumab should be administered at a dose of 8 mg/kg IV (8 mg/kg for patients ≥30 kg weight only; 12 mg/kg for patients <30 kg weight; doses exceeding 800 mg per infusion are not recommended); repeat every 8 hours as necessary (up to a maximum of four doses).
^{e} If the patient does not experience CRS during the next dose at the up to 50% reduced rate, the infusion rate can be increased to the initial rate for subsequent doses. However, if this patient experiences another CRS event, the infusion rate should be reduced by 25%-50% depending on the severity of the event.

### P. Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study drug to the end of treatment visit.

### Permitted Therapy

Patients are permitted to use the following therapies during the study:
Oral contraceptives with a failure rate of <1% per year.
Hormone-replacement therapy.
Hematopoietic growth factors.

Hematopoietic growth factors are allowed in accordance with instructions provided in local prescribing information, institutional guidelines, and/or published guidelines, and growth factor support (e.g., G-CSF) should be considered and administered per institutional guidelines for patients with Grade 3 or 4 neutropenia. The growth factors may include the following:
Erythropoietin.
Granulocyte colony-stimulating factor (filgrastim, pegfilgrastim).
Granulocyte/macrophage colony-stimulating factor (sargramostim).
Thrombopoietin (oprelvekin, eltrombopag).
Bisphosphonates or denosumab.

Bisphosphonates or denosumab are permitted as indicated for hypercalcemia or prevention of skeletal-related events and may be continued if the patient is already on either therapy prior to starting the study, or initiated during screening, pre-phase, or Cycle 1.

After Cycle 1, investigators should not prescribe bisphosphonates or denosumab to patients who have not received it before, unless it has been discussed and there is no sign of disease progression.
Tocilizumab reactively for CRS.
Anti-infective prophylaxis, as indicated for viral, fungal, bacterial, or pneumocystis infections prescribed per institutional practice. This may include IV immunoglobulin replacement therapy.
COVID-19 infection treatment or prophylaxis, as authorized or approved by local health authorities and per institutional practice.

Premedication with corticosteroids, antihistamines, and anti-pyretics are mandatory (above). Additional premedication may be administered at the discretion of the investigator.

In general, investigators should manage a patient's care with supportive therapies as clinically indicated, per local standard practice. Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion associated events manifested by dyspnea, hypotension, wheezing, bronchospasm, tachycardia, reduced oxygen saturation, or respiratory distress should be managed with supportive therapies as clinically indicated (e.g., supplemental oxygen and B2-adrenergic agonists).

### Cautionary Therapy

Medications Given with Precaution Due to Effects Related to CYP Enzymes

Given the expected pharmacology of cevostamab and elranatamab, the transient release of cytokines may suppress CYP450 enzymes and cause drug-drug interactions. In Study GO39775, cytokine levels are elevated during the first 24-48 hours following the administration of step-up and target doses of cevostamab in Cycle 1. Patients may be of highest risk of a drug-drug interaction are those receiving concomitant medications that are CYP450 substrates and have a narrow therapeutic index. Such concomitant medications should be monitored for toxicity, and dose adjusted accordingly.

Elranatamab has been shown to increase T-cell activation and induce cytokine production (including IL-2, IL-6, IL-10, TNF-□, and IFN-γ). Cytokines have been shown to modulate expression of CYP enzymes and transporters; therefore, elranatamab can potentially affect CYP enzyme and transporter expression levels, and consequently modulate the clearance of concomitant medications that are substrates for these enzymes or transporters. However, cytokine-mediated drug interactions observed in the clinic for other drugs have been modest, resulting in less than 2-fold changes in the exposure of co-administered small molecule drugs. At projected clinical doses, target-mediated drug disposition (TMDD) of elranatamab is expected. If concomitant medication alters target expression, it can potentially impact the PK of elranatamab.

### Herbal Therapies

Concomitant use of herbal therapies is not recommended because their pharmacokinetics, safety profiles, and potential drug-drug interactions are generally unknown. However, herbal therapies not intended for the treatment of cancer may be used during the study at the discretion of the investigator.

### Prohibited Therapy

Use of the following concomitant therapies is prohibited as described below:
Cytotoxic chemotherapy intended for treatment of MM.
Radiotherapy, with the following exceptions:
   Localized radiation therapy to a site of preexisting disease for palliation of symptoms may be allowed. Study treatment may be continued without interruption during the course of palliative radiation therapy if the Investigator believes it is in the best interest of the patient based on their benefit-risk assessment.
   If the patient develops a definite increase in the size of existing bone lesions or soft tissue plasmacytomas that meets the criteria for progressive disease, treatment must be discontinued for disease progression regardless of whether radiation therapy is initiated.
   Kyphoplasty, vertebroplasty, or emergency orthopedic surgery is permitted.
Immunotherapy for the treatment of cancer.
Hormone therapy for the treatment of cancer, whether approved by local regulatory authorities or investigational. Adjuvant endocrine therapy for non-metastatic, hormone-receptor positive breast cancer is permitted.
Biologic agents for the treatment of cancer.
Any therapies intended for the treatment of MM, whether approved by local regulatory authorities or investigational.

### Immunizations

Patients who participate in the study may not receive either primary or booster vaccination with live virus vaccines for at least 4 weeks before initiation of or at any time during study treatment.

### Example 3. Assessment of Safety

### A. Safety Plan

The safety plan for patients in the GO43979 study is based on clinical experience with cevostamab and elranatamab in completed and ongoing studies. The anticipated important safety risks for cevostamab and elranatamab are outlined below.

Several measures will be taken to ensure the safety of patients participating in this study. Eligibility criteria have been designed to exclude patients at higher risk for adverse events. Patients undergo safety monitoring during the study, including assessment of the nature, frequency, and severity of adverse events. In addition, guidelines for managing adverse events, including criteria for dosage modification and treatment interruption or discontinuation, are provided herein.

Risks Associated with Cevostamab

### Infusion-Related Reactions Cytokine Release Syndrome

The mechanism of action of cevostamab is immune cell-activation against FcRH5-expressing cells; therefore, a spectrum of events involving infusion-related reactions (IRRs), target-mediated cytokine release, and/or hypersensitivity with or without emergent ADAs may occur. Other bispecific antibody therapeutics involving T cell activation have been associated with IRR, cytokine release syndrome (CRS), and/or hypersensitivity reactions.

Cevostamab-related cytokine release has been observed in patients who received cevostamab monotherapy. Cytokine release has been transient and cytokine levels return to pre-study baseline within 24 hours. Transient cytokine release is expected based on the mechanism of T-cell engagement with plasma cells and C cells, resulting in T-cell activation and cytokine release.

Mild to moderate presentations of IRR and/or CRS may include symptoms such as fever, chills/rigors, tachycardia, headache, nausea, vomiting, diarrhea, and myalgia, and may be treated symptomatically with analgesics, antipyretics, and antihistamines as indicated. Severe or life-threatening presentations of IRR and/or CRS, with symptoms such as hypotension, tachycardia, dyspnea, tachypnea, organ dysfunction, rash and/or chest discomfort should be treated aggressively with supportive and resuscitative measures as indicated, including the use of high-dose corticosteroids, IV fluids, admission to intensive care unit, and other supportive measures per institutional practice. Severe CRS may be associated with other clinical sequelae such as disseminated intravascular coagulation, capillary leak syndrome, or macrophage activation syndrome/hemophagocytic lymphohistiocytosis (MAS/HLH). Standard of care for severe or life-threatening CRS resulting from immune-based therapy has not been established; case reports and recommendations using anti-cytokine therapy such as tocilizumab have been published (Teachey et al., Blood, 121: 5154-5157, 2013; Lee et al., Blood, 124: 1881-95, 2014; Maude et al., New Engl J Med, 371: 1507-1517, 2014).

The initial step-up dose of cevostamab given prior to administration of the cevostamab target dose is specifically intended to minimize risk of exaggerated cytokine release. Additionally, to further minimize the risk and sequelae of IRR and CRS, corticosteroid premedication will be required as described in Example 2 and cevostamab is administered in a clinical setting over a minimum of 4 hours for the step-up doses and for the first target dose. Patients are monitored closely following study drug administration with frequent vital-sign measurements to identify potential IRRs and/or CRS.

Macrophage activation syndrome and hemophagocytic lymphohistiocytosis HLH/MAS is a rare condition characterized by inappropriate immune activation, often precipitated by factors such as infection including EBV, autoimmune disease, and malignancies, including, very rarely, MM (Machaczka et al. 2011; Ramos-Casals et al. 2014; Bhatt et al. 2019; La Rosée et al. 2019; Mendes et al. 2020). HLH/MAS has also been reported with T-cell engaging therapies such as bispecific antibodies and CAR T-cell therapies (Teachey et al. 2013; Sandler et al. 2020; Kennedy et al. 2021; Setiadi et al. 2022; ABECMA® [idecabtagene vicleucel] US Prescribing Information [USPI]; BLINCYTO® [blinatumomab] USPI; CARVYKTI™ [ciltacabtagene autoleucel] USPI).

Severe CRS and HLH/MAS can have overlapping presentations and symptoms. However, unlike CRS, HLH/MAS has only occurred after Cycle 1 in clinical trials with cevostamab. In addition, not all cases of HLH/MAS reported in clinical trials with cevostamab had preceding CRS. In general, patients in clinical trials with cevostamab who experienced HLH/MAS presented with fever, pancytopenia, elevated transaminases, and elevated ferritin levels. Investigators should have a high index of suspicion for HLH/MAS so that the appropriate workup and management are initiated in a timely manner. It remains unknown whether cevostamab treatment may further increase the risk of developing HLH/MAS in patients who have additional risk factors.

Diagnostic, monitoring and management guidelines for HLH/MAS are described. Signs and symptoms may be nonspecific and onset may initially appear subtle. Once HLH/MAS is suspected, priority should be placed on rapid evaluation for organ damage, with the goal of starting treatment as rapidly as possible once the diagnosis of HLH/MAS is made (McClain and Eckstein 2023).

Traditionally, diagnosis of HLH/MAS has used the HLH-2004 criteria, which requires at least five of the following eight criteria to be met: (1) fever ≥38.5° C.; (2) splenomegaly; (3) peripheral blood cytopenias affecting at least two cell lines (hemoglobin ≤9 g/dL, platelets ≤100,000/mL, and/or absolute neutrophil count ≤1000/mL); (4) fasting triglyceride ≥265 mg/dL and/or fibrinogen ≤150 mg/dL; (5) hemophagocytosis in bone marrow, spleen, lymph node, or liver; (6) low or absent NK cell activity; (7) ferritin >500 ng/mL; (8) elevated soluble interleukin 2 (IL-2) receptor (soluble CD25) two standard deviations above laboratory-specific normal range (McClain and Eckstein 2023).

Because of the overlapping features of CRS and underlying hematologic malignancies, these diagnostic criteria for HLH/MAS may be suboptimal in the setting of T-cell-redirecting therapy (Hines et al. 2023). While there is currently no universally accepted set of criteria for diagnosing HLH/MAS in this setting, guidelines have been proposed, which include elevated ferritin (>2×ULN or baseline, and/or rapidly rising), in the setting of a hyperinflammatory syndrome that is independent from CRS and immune effector cell-associated neurotoxicity syndrome (ICANS) (Neelapu et al. 2018; Kennedy et al. 2021; Hayden et al. 2022; Hines et al. 2023). Rather than using specific laboratory cutoffs, investigators are recommended to consider the above criteria in conjunction with a constellation of the following signs or symptoms presenting in patients, to make the diagnosis:
Acute or unexplained rise in ferritin, particularly if accompanied by evidence of liver injury (significant increases in transaminases and/or bilirubin), new onset or marked worsening of cytopenias, and/or evidence of coagulopathy
Signs of hyperinflammation (elevated CRP, ferritin, etc.) accompanying acute organ damage to the pulmonary, renal, neurologic, and/or other organ systems
Signs and symptoms of severe sepsis
Fever of unknown origin
Grade ≥3 CRS at onset
Late-onset CRS signs and symptoms, regardless of severity, that first present ≥72 hours following cevostamab administration
CRS symptoms, regardless of severity, that persist ≥24 hours following initial management and appear refractory to treatment

In all cases of suspected HLH/MAS, the Medical Monitor should be immediately contacted and further dosing of cevostamab should be withheld. If HLH/MAS is confirmed, permanently discontinue cevostamab.

### Example 4. Statistical Considerations and Analysis Plan for the GO43979 Study

No formal hypothesis testing is planned. Descriptive statistics are used to summarize the safety, pharmacokinetics, and/or clinical activity of cevostamab, elranatamab and tocilizumab (if applicable). All analyses are based on the safety-evaluable population, defined as all patients who receive any amount of study drug.

Continuous variables are summarized using means, standard deviations, medians, and ranges; categorical variables are presented using counts and percentages. Summaries are calculated overall and by cohort.

### A. Determination of Sample Size

The trial is not powered to detect statistically significant differences between doses. Instead, the sample size for the trial is based on the study design described in Example 2. The planned enrollment for the pre-phase dosing stage of the study is approximately 3-6 patients. The planned enrollment for the safety lead-in stage of the study is approximately 17-37 patients. The planned enrollment for the expansion arm of this study is 40-80 patients. In total, the study anticipates enrolling 60-120 patients. Continuous safety monitoring to pause or stop patient enrollment for unacceptable toxicity are performed in the expansion stage of the study.

The trial utilizes a standard 3+3 design based on criteria described in the protocol. Information on the probability of not observing a dose-limiting toxicity (DLT) in 3 patients or observing ≤1 DLT in 6 patients given different underlying DLT rates is provided herein.

Table 10 provides probabilities of seeing at least one adverse event among 20 patients for probabilities ranging from 0.01 to 0.2 (i.e., adverse event frequencies of 1%-20%).

TABLE 10
Probability of Safety-Signal Detection
with Expansion Cohorts of 20 Patients

| True Underlying | Probability of Observing |
|---|---|
| Probability of | at Least1 Event in |
| an Adverse Event | 20 Patients (%) |
| 0.01 | 18 |
| 0.05 | 64 |
| 0.10 | 88 |
| 0.15 | 96 |
| 0.20 | 99 |

Twenty patients is a reasonable size for the expansion cohort to obtain preliminary efficacy data of the estimate of the very good partial response (VGPR) rate. For example, with a sample size of 20 and an observed VGPR rate of 60%, the corresponding 90% confidence interval for the true response rate is 39%-78% and the expected number of responses is 12.

### B. Safety Analyses

The safety analyses include all patients who received any amount of study drug across all arms of the study. Safety is assessed through summaries of adverse events, changes in laboratory test results, changes in ECGs, changes in anti-drug antibodies (ADAs), and changes in vital signs.

Summaries are presented by cohort and overall.

### C. Pharmacokinetic Analyses

Individual and mean serum elranatamab and cevostamab concentration versus time data are tabulated and plotted by dose level. PK parameters of cevostamab and elranatamab (i.e., total exposure (AUC), maximum observed serum concentration (Cmax), minimum observed serum concentration (Cmin), clearance, volume of distribution at steady state) are derived when appropriate as data allow. Compartmental, non-compartmental, and/or population methods may be considered. Estimates for these parameters are tabulated and summarized (mean, standard deviation, coefficient of variation, median, minimum, and maximum). Other parameters, such as accumulation ratio, half-life, and dose proportionality, may also be calculated.

Additional PK analyses are conducted as appropriate.

### D. Efficacy Analyses

Efficacy analyses are based on the definitions of response according to IMWG Uniform Response Criteria and endpoint definitions in Table 4. Patients with missing or no response assessments are classified as non-responders.

Rate data is reported with Clopper-Pearson exact binomial confidence intervals. When appropriate, time-to-event data is summarized using Kaplan-Meier methodology.

### E. Immunogenicity Analyses

The immunogenicity analysis is performed for both cevostamab and elranatamab. The numbers and proportions of ADA-positive patients and ADA-negative patients at baseline (baseline prevalence) and after drug administration (postbaseline incidence) are summarized. The relationship between ADA status and safety, activity, PK, and biomarker endpoints may be analyzed and reported via descriptive statistics.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating a subject having a multiple myeloma (MM), the method comprising administering to the subject (i) an effective amount of a first bispecific antibody that specifically binds to fragment crystallizable receptor-like 5 (FcRH5) and cluster of differentiation 3 (CD3) and (ii) an effective amount of a second bispecific antibody that specifically binds to B-cell maturation antigen (BCMA) and CD3.

2. The method of claim 1, wherein the subject has relapsed or refractory (R/R) MM.

3. The method of claim 2, wherein the subject has diagnosis of R/R MM according to International Myeloma Working Group (IMWG) criteria.

4. The method of any one of claims 1-3, wherein the first bispecific antibody and the second bispecific antibody are administered to the subject in a dosing regimen comprising:
(i) a first phase comprising one or more dosing cycles;
(ii) a second phase comprising one or more dosing cycles; and, optionally,
(iii) a third phase comprising one or more dosing cycles.

5. The method of claim 4, wherein the dosing regimen comprises the first phase and the second phase but does not comprise the third phase.

6. The method of claim 4, wherein the dosing regimen comprises the first phase, the second phase, and the third phase.

7. The method of claim 4 or 6, wherein each dosing cycle of the first phase, the second phase, and/or the third phase is a 14-day dosing cycle.

8. The method of any one of claims 4-7, further comprising a pre-phase, prior to the first phase, comprising one or more dosing cycles.

9. The method of claim 8, wherein the pre-phase comprises one dosing cycle (C1).

10. The method of claim 9, wherein the C1 of the pre-phase is: (i) approximately a 15-day dosing cycle; or (ii) approximately a 22-day dosing cycle.

11. The method of claim 10, wherein the C1 of the pre-phase is approximately a 15-day dosing cycle.

12. The method of claim 11, wherein the pre-phase comprises administering the first bispecific antibody to the subject on Day 9, Day 10, or Day 11 of the C1.

13. The method of claim 12, wherein the first bispecific antibody is administered to the subject on Day 9, Day 10, or Day 11 of the C1 at a step-up dose of 3.6 mg.

14. The method of any one of claims 11-13, wherein the pre-phase comprises administering the second bispecific antibody to the subject on (i) Day 1; (ii) Day 3, Day 4, or Day 5; and (iii) Day 8.

15. The method of claim 14, wherein the second bispecific antibody is administered to the subject on Day 1 at a first step-up dose of 12 mg.

16. The method of claim 14 or 15, wherein the second bispecific antibody is administered to the subject on Day 3, Day 4, or Day 5 at a second step-up dose of 32 mg.

17. The method of any one of claims 14-16, wherein the second bispecific antibody is administered to the subject on Day 8 at a target dose of 76 mg.

18. The method of claim 10, wherein the C1 of the pre-phase is approximately a 22-day dosing cycle.

19. The method of claim 18, wherein the pre-phase comprises administering the first bispecific antibody to the subject on Day 16, Day 17, or Day 18 of the C1.

20. The method of claim 19, wherein the first bispecific antibody is administered to the subject on Day 16, Day 17, or Day 18 of the C1 at a step-up dose of 3.6 mg.

21. The method of any one of claims 18-20, wherein the pre-phase comprises administering the second bispecific antibody to the subject on (i) Day 1; (ii) Day 3, Day 4, or Day 5; (iii) Day 8; and (iv) Day 15.

22. The method of claim 21, wherein the second bispecific antibody is administered to the subject on Day 1 at a first step-up dose of 12 mg.

23. The method of claim 21 or 22, wherein the second bispecific antibody is administered to the subject on Day 3, Day 4, or Day 5 at a second step-up dose of 32 mg.

24. The method of any one of claims 21-23, wherein the second bispecific antibody is administered to the subject on Day 8 at a target dose of 76 mg.

25. The method of any one of claims 21-24, wherein the second bispecific antibody is administered to the subject on Day 15 at a target dose of 76 mg.

26. The method of any one of claims 6-25, wherein the first phase comprises at least one dosing cycle, at least two dosing cycles, at least three dosing cycles, at least four dosing cycles, or at least five dosing cycles.

27. The method of claim 26, wherein the first phase comprises a first 14-day dosing cycle (C1).

28. The method of claim 27, wherein a target dose of the first bispecific antibody is administered to the subject during the first phase.

29. The method of claim 28, wherein the first phase comprises administering the target dose of the first bispecific antibody to the subject on Day 2, Day 3, or Day 4 of the C1.

30. The method of claim 28 or 29, wherein the target dose of the first bispecific antibody is 60 mg.

31. The method of claim 28 or 29, wherein the target dose of the first bispecific antibody is 60 mg administered for C1 through C8 of the first phase every two weeks (Q2W), and thereafter the target dose of the first bispecific antibody is 60 mg administered every four weeks (Q4W).

32. The method of claim 28 or 29, wherein the target dose of the first bispecific antibody is 105 mg.

33. The method of claim 28 or 29, wherein the target dose of the first bispecific antibody is 105 mg administered for C1 through C8 of the first phase Q2W, and thereafter the target dose of the first bispecific antibody is 60 mg administered Q4W.

34. The method of claim 28 or 29, wherein the target dose of the first bispecific antibody is 132 mg.

35. The method of any one of claims 27-34, wherein a target dose of the second bispecific antibody is administered to the subject during the first phase.

36. The method of claim 35, wherein the first phase comprises administering the target dose of the second bispecific antibody to the subject on Day 1 of the C1.

37. The method of claim 35 or 36, wherein the first phase comprises administering the target dose of the second bispecific antibody to the subject on Day 1 and Day 8 of the C1.

38. The method of any one of claims 35-37, wherein the target dose of the second bispecific antibody is 76 mg.

39. The method of claim 35 or 36, wherein the target dose of the second bispecific antibody is 76 mg administered for C1 through C8 of the first phase Q2W, and thereafter the target dose of the second bispecific antibody is 76 mg administered Q4W.

40. The method of any one of claims 6-39, wherein the second phase comprises at least one dosing cycle, at least two dosing cycles, at least three dosing cycles, at least four dosing cycles, at least five dosing cycles, at least six dosing cycles, at least seven dosing cycles, at least eight dosing cycles, at least nine dosing cycles, at least ten dosing cycles, at least eleven dosing cycles, at least twelve dosing cycles, at least thirteen dosing cycles, at least fourteen dosing cycles, at least fifteen dosing cycles, at least sixteen dosing cycles, at least seventeen dosing cycles, at least eighteen dosing cycles, at least nineteen dosing cycles, at least twenty dosing cycles, at least twenty-one dosing cycles, at least twenty-two dosing cycles, at least twenty-three dosing cycles, at least twenty-four dosing cycles, or at least twenty-five dosing cycles.

41. The method of claim 40, wherein the second phase comprises a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25).

42. The method of claim 40 or 41, wherein each dosing cycle of the second phase is a 14-day dosing cycle.

43. The method of any one of claims 40-42, wherein a target dose of the first bispecific antibody is administered to the subject during the second phase.

44. The method of any one of claims 40-43, wherein the second phase comprises administering the target dose of the first bispecific antibody to the subject on Day 1 of each dosing cycle.

45. The method of claim 42 or 43, wherein the second phase comprises administering the target dose of the first bispecific antibody to the subject on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and/or the C25 of the second phase.

46. The method of any one of claims 43-45, wherein the target dose is 60 mg.

47. The method of any one of claim 43-45, wherein the target dose of the first bispecific antibody is 60 mg administered for C1 through C8 of the second phase Q2W, and thereafter the target dose of the first bispecific antibody is 60 mg administered Q4W.

48. The method of any one of claim 43-45, wherein the target dose of the first bispecific antibody is 105 mg.

49. The method of any one of claim 43-45, wherein the target dose of the first bispecific antibody is 105 mg administered for C1 through C8 of the second phase Q2W, and thereafter the target dose of the first bispecific antibody is 60 mg administered Q4W.

50. The method of any one of claims 43-45, wherein the target dose is 132 mg.

51. The method of any one of claims 40-50, wherein a target dose of the second bispecific antibody is administered to the subject during the second phase.

52. The method of any one of claims 40-51, wherein the second phase comprises administering the target dose of the second bispecific antibody to the subject on Day 1 of each dosing cycle.

53. The method of claim 52, wherein the second phase comprises administering the target dose of the second bispecific antibody to the subject on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and/or the C25 of the second phase.

54. The method of any one of claims 40-51, wherein the second phase comprises administering the target dose of the second bispecific antibody to the subject on Day 1 and Day 8 of each dosing cycle.

55. The method of claim 54, wherein the second phase comprises administering the target dose of the second bispecific antibody to the subject on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and/or the C25 of the second phase.

56. The method of claim 54 or 55, wherein the subject achieves an IWMG response category of partial response (PR) or better with a response persisting for at least two months, and the dosing frequency of the second bispecific antibody is changed to every two weeks (Q2W).

57. The method of claim 56, wherein the subject subsequently has an increase of disease burden not yet qualifying as progressive disease according to IWMG criteria, and the dosing frequency is changed to every week (QW).

58. The method of claim 45 or 53, wherein if after 6 months the subject achieves an IWMG response category of complete response (CR), and the dosing frequency of the first bispecific antibody and the second bispecific antibody is changed from every two weeks (Q2W) to every four weeks (Q4W).

59. The method of claim 53 wherein the subject achieves an IWMG response category of partial response (PR) with a response persisting for at least two months, and the dosing frequency of the second bispecific antibody is changed from every two weeks (Q2W) to every four weeks (Q4W).

60. The method of any one of claims 40-51, wherein the second phase comprises administering the target dose of the second bispecific antibody to the subject on Day 8 of each dosing cycle.

61. The method of claim 58, wherein the second phase comprises administering the target dose of the second bispecific antibody to the subject on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and/or the C25.

62. The method of any one of claims 51-61, wherein the target dose of the second bispecific antibody is 76 mg.

63. The method of any one of claims 51-59, wherein the target dose of the second bispecific antibody is 76 mg administered for C1 through C8 of the second phase Q2W, and thereafter the target dose of the second bispecific antibody is 76 mg administered Q4W.

64. The method of any one of claims 1-63, wherein the method comprises administering the first bispecific antibody to the subject until disease progression, unacceptable toxicity, or up to 26 total dosing cycles.

65. The method of any one of claims 4 and 46-56, wherein the third phase comprises one or more dosing cycles.

66. The method of claim 65, wherein the third phase comprises between 1 and 130 dosing cycles.

67. The method of claim 65 or 66, wherein each dosing cycle of the third phase is a 14-day dosing cycle.

68. The method of any one of claims 65-67, wherein a target dose of the second bispecific antibody is administered to the subject during the third phase.

69. The method of claim 68, wherein the third phase comprises administering the target dose of the second bispecific antibody to the subject on Day 1 of each dosing cycle of the third phase.

70. The method of claim 68, wherein the third phase comprises administering the target dose of the second bispecific antibody to the subject on Day 1 and Day 8 of each dosing cycle of the third phase.

71. The method of claim 70, wherein the subject achieves an IWMG response category of partial response (PR) or better with a response persisting for at least two months, and the dosing frequency of the second bispecific antibody is changed to every two weeks (Q2W).

72. The method of claim 69 or 71, wherein if after 6 months the subject achieves an IWMG response category of complete response (CR), and the dosing frequency of the first bispecific antibody and the second bispecific antibody is changed from every two weeks (Q2W) to every four weeks (Q4W).

73. The method of claim 69 or 71, wherein the subject achieves an IWMG response category of partial response (PR) with a response persisting for at least two months, and the dosing frequency of the second bispecific antibody is changed from every two weeks (Q2W) to every four weeks (Q4W).

74. The method of claim 71, wherein the subject subsequently has an increase of disease burden not yet qualifying as progressive disease according to IWMG criteria, and the dosing frequency is changed to every week (QW).

75. The method of any one of claims 68-71, wherein the target dose of the second bispecific antibody is 76 mg.

76. The method of any one of claims 1-75, wherein the second bispecific antibody is administered to the subject until disease progression or unacceptable toxicity.

77. The method of any one of claims 1-76, wherein the first bispecific antibody is administered to the subject intravenously.

78. The method of any one of claims 1-77, wherein the second bispecific antibody is administered to the subject subcutaneously.

79. The method of any one of claims 1-78, further comprising administering a corticosteroid to the subject.

80. The method of any one of claims 6-79, further comprising administering a corticosteroid to the subject during the first phase, the second phase, and/or the third phase.

81. The method of any one of claims 6-80, wherein the corticosteroid is administered to the subject during the first phase 1 hour (±15 minutes) before administration of the first bispecific antibody or the second bispecific antibody.

82. The method of any one of claims 6-81, wherein:
(i) the subject experienced cytokine release syndrome (CRS) with a prior administration of the first bispecific antibody or the second bispecific antibody, and the corticosteroid is administered to the subject during the second phase 1 hour (±15 minutes) before administration of the first bispecific antibody or the second bispecific antibody; and/or
(ii) the subject experienced CRS with a prior administration of the first bispecific antibody or the second bispecific antibody, and the corticosteroid is administered to the subject during the third phase 1 hour (±15 minutes) before administration of the second bispecific antibody.

83. The method of any one of claims 8-82, further comprising administering a corticosteroid to the subject during the pre-phase.

84. The method of any one of claims 11-83, wherein the corticosteroid is administered to the subject during the C1 of the pre-phase 1 hour (±15 minutes) before administration of the first bispecific antibody or the second bispecific antibody.

85. The method of any one of claims 79-84, wherein the corticosteroid is dexamethasone or methylprednisolone.

86. The method of claim 85, wherein the corticosteroid is dexamethasone.

87. The method of claim 85 or 86, wherein the dexamethasone is administered to the subject at a dose of about 20 mg.

88. The method of claim 85, wherein the methylprednisolone is administered to the subject at a dose of about 80 mg.

89. The method of any one of claims 79-88, wherein the corticosteroid is administered to the subject intravenously.

90. The method of any one of claims 1-89 and 175-190, wherein the first bispecific antibody that specifically binds to FcRH5 and CD3 comprises an anti-FcRH5 arm comprising a first binding domain comprising the following six hypervariable regions (HVRs):
(a) an HVR-H1 comprising the amino acid sequence of RFGVH (SEQ ID NO: 1);
(b) an HVR-H2 comprising the amino acid sequence of VIWRGGSTDYNAAFVS (SEQ ID NO: 2);
(c) an HVR-H3 comprising the amino acid sequence of HYYGSSDYALDN (SEQ ID NO: 3);
(d) an HVR-L1 comprising the amino acid sequence of KASQDVRNLVV (SEQ ID NO: 4);
(e) an HVR-L2 comprising the amino acid sequence of SGSYRYS (SEQ ID NO: 5); and
(f) an HVR-L3 comprising the amino acid sequence of QQHYSPPYT (SEQ ID NO: 6).

91. The method of any one of claims 1-78, wherein the first bispecific antibody that specifically binds to FcRH5 and CD3 comprises an anti-FcRH5 arm comprising a first binding domain comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b).

92. The method of claim 91, wherein the first binding domain comprises a VH domain comprising an amino acid sequence of SEQ ID NO: 7 and a VL domain comprising an amino acid sequence of SEQ ID NO: 8.

93. The method of any one of claims 1-92 and 175-190, wherein the first bispecific antibody that specifically binds to FcRH5 and CD3 comprises an anti-CD3 arm comprising a second binding domain comprising the following six HVRs:
(a) an HVR-H1 comprising the amino acid sequence of SYYIH (SEQ ID NO: 9);
(b) an HVR-H2 comprising the amino acid sequence of WIYPENDNTKYNEKFKD (SEQ ID NO: 10);
(c) an HVR-H3 comprising the amino acid sequence of DGYSRYYFDY (SEQ ID NO: 11);
(d) an HVR-L1 comprising the amino acid sequence of KSSQSLLNSRTRKNYLA (SEQ ID NO: 12);
(e) an HVR-L2 comprising the amino acid sequence of WTSTRKS (SEQ ID NO: 13); and
(f) an HVR-L3 comprising the amino acid sequence of KQSFILRT (SEQ ID NO: 14).

94. The method of any one of claims 1-93, wherein the first bispecific antibody that specifically binds to FcRH5 and CD3 comprises an anti-CD3 arm comprising a second binding domain comprising (a) a VH domain comprising an amino acid sequence having at least 95% sequence identity to the amino acid sequence of SEQ ID NO: 15; (b) a VL domain comprising an amino acid sequence having at least 95% sequence identity to the amino acid sequence of SEQ ID NO: 16; or (c) a VH domain as in (a) and a VL domain as in (b).

95. The method of claim 94, wherein the second binding domain comprises a VH domain comprising an amino acid sequence of SEQ ID NO: 15 and a VL domain comprising an amino acid sequence of SEQ ID NO: 16.

96. The method of any one of claims 1-95 and 175-190, wherein the first bispecific antibody that specifically binds to FcRH5 and CD3 comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein:
(a) H1 comprises the amino acid sequence of SEQ ID NO: 35;
(b) L1 comprises the amino acid sequence of SEQ ID NO: 36;
(c) H2 comprises the amino acid sequence of SEQ ID NO: 37; and
(d) L2 comprises the amino acid sequence of SEQ ID NO: 38.

97. The method of any one of claims 1-96, wherein the first bispecific antibody that specifically binds to FcRH5 and CD3 comprises an aglycosylation site mutation.

98. The method of claim 97, wherein the aglycosylation site mutation reduces effector function of the bispecific antibody.

99. The method of claim 98, wherein the aglycosylation site mutation is a substitution mutation.

100. The method of any one of claims 1-99, wherein the first bispecific antibody that specifically binds to FcRH5 and CD3 comprises a substitution mutation in the Fc region that reduces effector function.

101. The method of any one of claims 1-100, wherein the first bispecific antibody that specifically binds to FcRH5 and CD3 is a monoclonal antibody.

102. The method of any one of claims 1-101, wherein the first bispecific antibody that specifically binds to FcRH5 and CD3 is a chimeric antibody.

103. The method of any one of claims 1-102, wherein the first bispecific antibody that specifically binds to FcRH5 and CD3 is a humanized antibody.

104. The method of any one of claims 1-95 and 97-103, wherein the first bispecific antibody that specifically binds to FcRH5 and CD3 is an antibody fragment that specifically binds FcRH5 and CD3.

105. The method of claim 104, wherein the antibody fragment that specifically binds FcRH5 and CD3 is selected from the group consisting of Fab, Fab′-SH, Fv, scFv, and (Fab′)2 fragments.

106. The method of any one of claims 1-103, wherein the first bispecific antibody that specifically binds to FcRH5 and CD3 is a full-length antibody.

107. The method of any one of claims 1-106, wherein the first bispecific antibody that specifically binds to FcRH5 and CD3 is an IgG antibody.

108. The method of claim 107, wherein the IgG antibody that specifically binds to FcRH5 and CD3 is an IgG1 antibody.

109. The method of any one of claims 1-108, wherein the first bispecific antibody that specifically binds to FcRH5 and CD3 comprises one or more heavy chain constant domains, wherein the one or more heavy chain constant domains are selected from a first CH1 (CH11) domain, a first CH2 (CH21) domain, a first CH3 (CH31) domain, a second CH1 (CH12) domain, second CH2 (CH22) domain, and a second CH3 (CH32) domain.

110. The method of claim 109, wherein at least one of the one or more heavy chain constant domains is paired with another heavy chain constant domain.

111. The method of claim 110, wherein the CH31 and CH32 domains each comprise a protuberance or cavity, and wherein the protuberance or cavity in the CH31 domain is positionable in the cavity or protuberance, respectively, in the CH32 domain.

112. The method of claim 111, wherein the CH31 and CH32 domains meet at an interface between the protuberance and cavity.

113. The method of any one of claims 109-112, wherein the CH21 and CH22 domains each comprise a protuberance or cavity, and wherein the protuberance or cavity in the CH21 domain is positionable in the cavity or protuberance, respectively, in the CH22 domain.

114. The method of claim 113, wherein the CH21 and CH22 domains meet at an interface between said protuberance and cavity.

115. The method of claim 114, wherein the anti-FcRH5 arm comprises the protuberance and the anti-CD3 arm comprises the cavity.

116. The method of claim 115, wherein a CH3 domain of the anti-FcRH5 arm comprises a protuberance comprising a T366W amino acid substitution mutation (EU numbering) and a CH3 domain of the anti-CD3 arm comprises a cavity comprising T366S, L368A, and Y407V amino acid substitution mutations (EU numbering).

117. The method of any one of claims 1-103 and 106-116, wherein the first bispecific antibody that specifically binds to FcRH5 and CD3 is cevostamab.

118. The method of any one of claims 1-117 and 175-190, wherein the second bispecific antibody that specifically binds to BCMA and CD3 comprises an anti-BCMA arm comprising a first binding domain comprising the following six HVRs:
(a) an HVR-H1 comprising the amino acid sequence of GFTFSSY (SEQ ID NO: 40), SYPMS (SEQ ID NO: 39), or GFTFSSYPMS (SEQ ID NO: 41);
(b) an HVR-H2 comprising the amino acid sequence of GGSGGS (SEQ ID NO: 43) or AIGGSGGSLPYADIVKG (SEQ ID NO: 42);
(c) an HVR-H3 comprising the amino acid sequence of YWPMDI (SEQ ID NO: 44);
(d) an HVR-L1 comprising the amino acid sequence of RASQSVSSSYLA (SEQ ID NO: 45);
(e) an HVR-L2 comprising the amino acid sequence of DASIRAT (SEQ ID NO: 46); and
(f) an HVR-L3 comprising the amino acid sequence of QQYQSWPLT (SEQ ID NO: 47).

119. The method of any one of claims 1-118, wherein the second bispecific antibody that specifically binds to BCMA and CD3 comprises an anti-BCMA arm comprising a first binding domain comprising (a) a VH domain comprising an amino acid sequence having at least 95% sequence identity to the amino acid sequence of SEQ ID NO: 48; (b) a VL domain comprising an amino acid sequence having at least 95% sequence identity to the amino acid sequence of SEQ ID NO: 49; or (c) a VH domain as in (a) and a VL domain as in (b).

120. The method of claim 119, wherein the first binding domain of the second bispecific antibody comprises a VH domain comprising an amino acid sequence of SEQ ID NO: 48 and a VL domain comprising an amino acid sequence of SEQ ID NO: 49.

121. The method of any one of claims 1-120 and 175-190, wherein the second bispecific antibody that specifically binds to BCMA and CD3 comprises an anti-CD3 arm comprising a second binding domain comprising the following six HVRs:
(a) an HVR-H1 comprising the amino acid sequence of GFTFSDY (SEQ ID NO: 53), DYYMT (SEQ ID NO: 52), or GFTFSDYYMT (SEQ ID NO: 54);
(b) an HVR-H2 comprising the amino acid sequence of RNRARGYT (SEQ ID NO: 56) or FIRNRARGYTSDHNPSVKG (SEQ ID NO: 55);
(c) an HVR-H3 comprising the amino acid sequence of DRPSYYVLDY (SEQ ID NO: 57);
(d) an HVR-L1 comprising the amino acid sequence of KSSQSLFNVRSRKNYLA (SEQ ID NO: 58);
(e) an HVR-L2 comprising the amino acid sequence of WASTRES (SEQ ID NO: 59); and
(f) an HVR-L3 comprising the amino acid sequence of KQSYDLFT (SEQ ID NO: 60).

122. The method of any one of claims 1-121, wherein the second bispecific antibody that specifically binds to BCMA and CD3 comprises an anti-CD3 arm comprising a second binding domain comprising (a) a VH domain comprising an amino acid sequence having at least 95% sequence identity to the amino acid sequence of SEQ ID NO: 61; (b) a VL domain comprising an amino acid sequence having at least 95% sequence identity to the amino acid sequence of SEQ ID NO: 62; or (c) a VH domain as in (a) and a VL domain as in (b).

123. The method of claim 122, wherein the second binding domain of the second bispecific antibody comprises a VH domain comprising an amino acid sequence of SEQ ID NO: 61 and a VL domain comprising an amino acid sequence of SEQ ID NO: 62.

124. The method of any one of claims 1-123 and 175-190, wherein the bispecific antibody that specifically binds to BCMA and CD3 comprises an anti-BCMA arm comprising a heavy chain polypeptide (H1) and a light chain polypeptide (L1) and an anti-CD3 arm comprising a heavy chain polypeptide (H2) and a light chain polypeptide (L2), and wherein:
(a) H1 comprises the amino acid sequence of SEQ ID NO: 50;
(b) L1 comprises the amino acid sequence of SEQ ID NO: 51;
(c) H2 comprises the amino acid sequence of SEQ ID NO: 63; and
(d) L2 comprises the amino acid sequence of SEQ ID NO: 64.

125. The method of any one of claims 1-124, wherein the second bispecific antibody comprises a substitution mutation in the Fc region that reduces effector function.

126. The method of any one of claims 1-125, wherein the second bispecific antibody is a monoclonal, chimeric, or humanized antibody.

127. The method of any one of claims 1-123, 125, and 126, wherein the second bispecific antibody that specifically binds to BCMA and CD3 is an antibody fragment that specifically binds BCMA and CD3.

128. The method of claim 127, wherein the antibody fragment that specifically binds BCMA and CD3 is selected from the group consisting of Fab, Fab′-SH, Fv, scFv, and (Fab′)2 fragments.

129. The method of any one of claims 1-126, wherein the second bispecific antibody is a full-length antibody.

130. The method of any one of claims 1-129, wherein the second bispecific antibody is an IgG antibody.

131. The method of claim 130, wherein the IgG antibody is an IgG2 antibody.

132. The method of any one of claims 1-123 and 125-131, wherein the second bispecific antibody comprises first and second respective human IgG2 constant regions comprising amino acid modifications at positions 223 and 228, or 223, 225 and 228, respectively, in the hinge region, and at position 368 or 409, respectively, in the CH3 region (EU numbering).

133. The method of claim 132, wherein the first human IgG2 constant region comprises C223E, P228E, and L368E amino acid substitution mutations (EU numbering).

134. The method of claim 132 or 133, wherein the second human IgG2 constant region comprises C223R, E225R, P228R, and K409R amino acid substitution mutations (EU numbering).

135. The method of any one of claims 1-126 and 129-134, wherein the second bispecific antibody that specifically binds to BCMA and CD3 is elranatamab.

136. The method of any one of claims 1-135, wherein the first bispecific antibody and/or the second bispecific antibody are administered to the subject concurrently with one or more additional therapeutic agents.

137. The method of any one of claims 1-135, wherein the first bispecific antibody and/or the second bispecific antibody are administered to the subject prior to the administration of one or more additional therapeutic agents.

138. The method of any one of claims 1-135, wherein the first bispecific antibody and/or the second bispecific antibody is administered to the subject subsequent to the administration of one or more additional therapeutic agents.

139. The method of any one of claims 136-138, wherein the one or more additional therapeutic agents comprise an effective amount of an anti-IL-6 antibody.

140. The method of claim 139, wherein the anti-IL-6 antibody is tocilizumab.

141. The method of any one of claims 1-140, wherein the subject has a CRS event, and the method further comprises treating the symptoms of the CRS event while suspending treatment with the first bispecific antibody and the second bispecific antibody.

142. The method of claim 141, wherein the method further comprises administering to the subject an effective amount of tocilizumab to treat the CRS event.

143. The method of claim 142, wherein the CRS event does not resolve or worsens within 24 hours of treating the symptoms of the CRS event, and the method further comprising administering to the subject one or more additional doses of tocilizumab to manage the CRS event.

144. The method of any one of claims 139, 140, 142, and 143, wherein tocilizumab is administered to the subject by intravenous infusion.

145. The method of claim 144, wherein:
(a) the subject weighs ≥30 kg, and tocilizumab is administered to the subject at a dose of 8 mg/kg;
or
(b) the subject weighs <30 kg, and tocilizumab is administered to the subject at a dose of 12 mg/kg.

146. The method of any one of claims 139, 140, 142, and 143, wherein tocilizumab is administered to the subject 2 hours before administration of the first bispecific antibody and/or the second bispecific antibody.

147. The method of any one of claims 136-146, wherein the one or more additional therapeutic agents comprise an effective amount of acetaminophen or paracetamol.

148. The method of claim 139, wherein acetaminophen or paracetamol is administered to the subject at a dose of between 500 mg to 1000 mg.

149. The method of claim 139 or 140, wherein acetaminophen or paracetamol is administered to the subject orally.

150. The method of any one of claims 136-146, wherein the one or more additional therapeutic agents comprise an effective amount of diphenhydramine.

151. The method of claim 150, wherein diphenhydramine is administered to the subject at a dose of between 25 mg to 50 mg.

152. The method of claim 150 or 151, wherein diphenhydramine is administered to the subject orally.

153. The method of any one of claims 138-146, wherein the method comprises pre-medication with the following agents prior to administration of the first bispecific antibody and/or the second bispecific antibody to the subject: (i) a corticosteroid; (ii) acetaminophen or paracetamol; and/or (iii) diphenhydramine.

154. The method of claim 153, wherein the method comprises a pre-phase, and the corticosteroid is administered to the subject 1 hour (±15 minutes) prior to any administration of the first bispecific antibody and/or the second bispecific antibody during the pre-phase.

155. The method of claim 153 or 154, wherein the method comprises a first phase, and the corticosteroid is administered to the subject 1 hour (±15 minutes) prior to any administration of the first bispecific antibody and/or the second bispecific antibody during the first phase.

156. The method of any one of claims 153-155, wherein the method comprises a second phase, wherein the subject has experienced CRS with a prior administration of the first bispecific antibody and/or the second bispecific antibody, and the corticosteroid is administered to the subject 1 hour (±15 minutes) prior to any administration of the first bispecific antibody and/or the second bispecific antibody during the second phase.

157. The method of any one of claims 153-156, wherein the method comprises a third phase, wherein the subject has experienced CRS with a prior administration of the first bispecific antibody and/or the second bispecific antibody, and the corticosteroid is administered to the subject 1 hour (±15 minutes) prior to any administration of the second bispecific antibody during the third phase.

158. The method of any one of claims 153-157, wherein the corticosteroid is dexamethasone or methylprednisolone.

159. The method of claim 158, wherein the corticosteroid is dexamethasone.

160. The method of claim 158 or 159, wherein the dexamethasone is administered to the subject at a dose of about 20 mg.

161. The method of claim 158, wherein the methylprednisolone is administered to the subject at a dose of about 80 mg.

162. The method of any one of claims 153-161, wherein the corticosteroid is administered to the subject intravenously.

163. The method of any one of claims 153-162, wherein acetaminophen or paracetamol is administered to the subject at a dose of between 500 mg to 1000 mg.

164. The method of any one of claims 153-163, wherein acetaminophen or paracetamol is administered to the subject orally.

165. The method of any one of claims 153-164, wherein diphenhydramine is administered to the subject at a dose of between 25 mg to 50 mg.

166. The method of any one of claims 153-165, wherein diphenhydramine is administered to the subject orally.

167. A method of treating a subject having an R/R MM, the method comprising administering to the subject cevostamab and elranatamab in a dosing regimen comprising:
(i) a pre-phase comprising an approximately 15-day dosing cycle (C1);
(ii) a first phase, following the pre-phase, comprising a first dosing cycle (C1), wherein the C1 of the first phase is a 14-day dosing cycle;
(iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and
(iv) a third phase, following the second phase, comprising one or more dosing cycles, wherein cevostamab is administered to the subject:
at a step-up dose of 3.6 mg on Day 9, Day 10, or Day 11 of the C1 of the pre-phase; at a target dose of 60 mg on Day 2, Day 3, or Day 4 of the C1 of the first phase; and at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and wherein elranatamab is administered to the subject:
at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase;
at a target dose of 76 mg on Day 1 of the C1 of the first phase;
at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

168. A method of treating a subject having an R/R MM, the method comprising administering to the subject cevostamab and elranatamab in a dosing regimen comprising:
(i) a pre-phase comprising an approximately 22-day dosing cycle (C1);
(ii) a first phase, following the pre-phase, comprising a first dosing cycle (C1), wherein the C1 of the first phase is a 14-day dosing cycle;
(iii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and
(iv) a third phase, following the second phase, comprising one or more dosing cycles,
wherein cevostamab is administered to the subject:
at a step-up dose of 3.6 mg on Day 16, Day 17, or Day 18 of the C1 of the pre-phase;
at a target dose of 60 mg on Day 2, Day 3, or Day 4 of the C1 of the first phase; and
at a target dose of 60 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and
wherein elranatamab is administered to the subject:
at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase;
at a target dose of 76 mg on Day 1 of the C1 of the first phase;
at a target dose of 76 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

169. A method of treating a subject having an R/R MM, the method comprising administering to the subject cevostamab and elranatamab in a dosing regimen comprising:
(i) a first phase comprising a first dosing cycle (C1), wherein the C1 of the first phase is a 14-day dosing cycle;
(ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and
(iii) a third phase, following the second phase, comprising one or more dosing cycles,
wherein cevostamab is administered to the subject:
at a target dose of 60 mg, 105 mg, or 132 mg on Day 2, Day 3, or Day 4 of the C1 of the first phase; and
at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and
wherein elranatamab is administered to the subject:
at a target dose of 76 mg on Day 1 and Day 8 of the C1 of the first phase;
at a target dose of 76 mg on Day 1 and Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 and Day 8 of each of the one or more dosing cycles of the third phase.

170. A method of treating a subject having an R/R MM, the method comprising administering to the subject cevostamab and elranatamab in a dosing regimen comprising:
(i) a first phase comprising a first dosing cycle (C1), wherein the C1 of the first phase is a 14-day dosing cycle;
(ii) a second phase, following the first phase, comprising a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), a thirteenth dosing cycle (C13), a fourteenth dosing cycle (C14), a fifteenth dosing cycle (C15), a sixteenth dosing cycle (C16), a seventeenth dosing cycle (C17), an eighteenth dosing cycle (C18), a nineteenth dosing cycle (C19), a twentieth dosing cycle (C20), a twenty-first dosing cycle (C21), a twenty-second dosing cycle (C22), a twenty-third dosing cycle (C23), a twenty-fourth dosing cycle (C24), and a twenty-fifth dosing cycle (C25), wherein each dosing cycle of the second phase is a 14-day dosing cycle; and
(iii) a third phase, following the second phase, comprising one or more dosing cycles,
wherein cevostamab is administered to the subject:
at a target dose of 60 mg, 105 mg, or 132 mg on Day 2, Day 3, or Day 4 of the C1 of the first phase; and at a target dose of 60 mg, 105 mg, or 132 mg on Day 1 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and
wherein elranatamab is administered to the subject:
at a target dose of 76 mg on Day 1 and Day 8 of the C1 of the first phase;
at a target dose of 76 mg on Day 8 of the C1, the C2, the C3, the C4, the C5, the C6, the C7, the C8, the C9, the C10, the C11, the C12, the C13, the C14, the C15, the C16, the C17, the C18, the C19, the C20, the C21, the C22, the C23, the C24, and the C25 of the second phase; and at a target dose of 76 mg on Day 1 of each of the one or more dosing cycles of the third phase.

171. The method of claim 169 or 170, further comprising a pre-phase prior to the first phase.

172. The method of claim 171, wherein the pre-phase comprises an approximately 15-day dosing cycle (C1), wherein:
cevostamab is administered to the subject at a step-up dose of 3.6 mg on Day 9, 10, or 11 of the C1 of the pre-phase; and
elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 of the C1 of the pre-phase.

173. The method of claim 171, wherein the pre-phase comprises an approximately 22-day dosing cycle (C1), wherein:
cevostamab is administered to the subject at a step-up dose of 3.6 mg on Day 16, Day 17, or Day 18 of the C1 of the pre-phase; and elranatamab is administered to the subject at a first step-up dose of 12 mg on Day 1 of the C1 of the pre-phase; at a second step-up dose of 32 mg on Day 3, Day 4, or Day 5 of the C1 of the pre-phase; and at a target dose of 76 mg on Day 8 and Day 15 of the C1 of the pre-phase.

174. The method of any one of claims 1-161 and 163-178, wherein the subject is a human subject.

175. The method of claim 4 or 5, wherein each dosing cycle (C) of the second phase is a 28-day dosing cycle.

176. The method of claim 175, wherein the dosing cycle of the first phase is a 14-day dosing cycle.

177. The method of claim 175 or 176, wherein the first phase includes dosing cycles C1 to C26, and the second phase comprises dosing cycles C27 and beyond.

178. The method of claim 177, wherein the second bispecific antibody is administered to the subject on Day 1 of each dosing cycle of the second phase at a target dose of 76 mg.

179. The method of any one of claims 1-3, wherein each dosing cycle after at about one year from a first dosing of the second bispecific antibody administered to the subject is 28-days.

180. The method of any one of claims 1-3, wherein each dosing cycle of the second bispecific antibody administered to the subject on the 27^{th} dosing cycle and thereafter is a 28-days.

181. The method of claim 180, wherein each dosing cycle C1 to C26 is a 14-day dosing cycle.

182. The method of claim 6, wherein each dosing cycle of the first phase and the second phase is a 14-day dosing cycle, and each dosing cycle of the third phase is a 28-day dosing cycle.

183. The method of claim 182, wherein the second bispecific antibody is administered to the subject on Day 1 and Day 8 of each dosing cycle of the first phase.

184. The method of claim 182, wherein the second bispecific antibody is administered to the subject on Day 1 of each dosing cycle of the second phase.

185. The method of claim 182, wherein the second bispecific antibody is administered to the subject on Day 1 of each dosing cycle of the third phase.

186. The method of claim 182, wherein the second bispecific antibody is administered to the subject on Day 1 and Day 8 of each dosing cycle of the first phase; wherein the second bispecific antibody is administered to the subject on Day 1 of each dosing cycle of the second phase; and wherein the second bispecific antibody is administered to the subject on Day 1 of each dosing cycle of the third phase.

187. The method of any one of claims 182-186, wherein each second bispecific antibody is administered to the subject at a target dose of 76 mg.

188. The method of any one of claims 182-187, wherein the third phase starts about one year from the first target dose of the second bispecific antibody administered to the subject.

189. The method of any one of claims 182-187, wherein 27^{th} dosing cycle (C27) is the first dosing cycle of the third phase.

190. The method of claim 8 or 14, wherein the pre-phase comprises administering the second bispecific antibody to the subject on (i) Day 1; (ii) Day 4; and (iii) Day 8.


SEQUENCE LISTING (64 sequences)

SEQ ID NO: 1 - protein, 5 aa, synthetic construct
RFGVH

SEQ ID NO: 2 - protein, 16 aa, synthetic construct
VIWRGGSTDYNAAFVS

SEQ ID NO: 3 - protein, 12 aa, synthetic construct
HYYGSSDYALDN

SEQ ID NO: 4 - protein, 11 aa, synthetic construct
KASQDVRNLVV

SEQ ID NO: 5 - protein, 7 aa, synthetic construct
SGSYRYS

SEQ ID NO: 6 - protein, 9 aa, synthetic construct
QQHYSPPYT

SEQ ID NO: 7 - protein, 120 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSS

SEQ ID NO: 8 - protein, 107 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIK

SEQ ID NO: 9 - protein, 5 aa, synthetic construct
SYYIH

SEQ ID NO: 10 - protein, 17 aa, synthetic construct
WIYPENDNTKYNEKFKD

SEQ ID NO: 11 - protein, 10 aa, synthetic construct
DGYSRYYFDY

SEQ ID NO: 12 - protein, 17 aa, synthetic construct
KSSQSLLNSRTRKNYLA

SEQ ID NO: 13 - protein, 7 aa, synthetic construct
WTSTRKS

SEQ ID NO: 14 - protein, 8 aa, synthetic construct
KQSFILRT

SEQ ID NO: 15 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 17 - protein, 30 aa, synthetic construct
EVQLVESGPGLVKPSETLSLTCTVSGFSLT

SEQ ID NO: 18 - protein, 14 aa, synthetic construct
WVRQPPGKGLEWLG

SEQ ID NO: 19 - protein, 32 aa, synthetic construct
RLTISKDNSKNQVSLKLSSVTAADTAVYYCSN

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WFQQKPGKAPKLLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGFTFT

SEQ ID NO: 26 - protein, 14 aa, synthetic construct
WVRQAPGQGLEWIG

SEQ ID NO: 27 - protein, 32 aa, synthetic construct
RVTITADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQSPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - DNA, 5620 bp, Homo sapiens
  source 1..5620: mol_type = genomic DNA
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 34 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 35 - protein, 450 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 449 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 219 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 5 aa, synthetic construct
SYPMS

SEQ ID NO: 40 - protein, 7 aa, synthetic construct
GFTFSSY

SEQ ID NO: 41 - protein, 10 aa, synthetic construct
GFTFSSYPMS

SEQ ID NO: 42 - protein, 17 aa, synthetic construct
AIGGSGGSLPYADIVKG

SEQ ID NO: 43 - protein, 6 aa, synthetic construct
GGSGGS

SEQ ID NO: 44 - protein, 6 aa, synthetic construct
YWPMDI

SEQ ID NO: 45 - protein, 12 aa, synthetic construct
RASQSVSSSYLA

SEQ ID NO: 46 - protein, 7 aa, synthetic construct
DASIRAT

SEQ ID NO: 47 - protein, 9 aa, synthetic construct
QQYQSWPLT

SEQ ID NO: 48 - protein, 115 aa, synthetic construct
EVQLLESGGGLVQPGGSLRLSCAASGFTFSSYPMSWVRQAPGKGLEWVSAIGGSGGSLPYADIVKGRFTISRDNSKNTLYLQMNSLRAEDTAVYYCARYWPMDIWGQGTLVTVSS

SEQ ID NO: 49 - protein, 108 aa, synthetic construct
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIK

SEQ ID NO: 50 - protein, 441 aa, synthetic construct
EVQLLESGGGLVQPGGSLRLSCAASGFTFSSYPMSWVRQAPGKGLEWVSAIGGSGGSLPYADIVKGRFTISRDNSKNTLYLQMNSLRAEDTAVYYCARYWPMDIWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCEVECPECPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCEVKGFYPSDIAVEWESNGQPENNYKTTPPMLDSDGSFFLYSKLTVDKSRWQQGNVFSCSVMHEALHNHYTQKSLSLSPGK

SEQ ID NO: 51 - protein, 215 aa, synthetic construct
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 5 aa, synthetic construct
DYYMT

SEQ ID NO: 53 - protein, 7 aa, synthetic construct
GFTFSDY

SEQ ID NO: 54 - protein, 10 aa, synthetic construct
GFTFSDYYMT

SEQ ID NO: 55 - protein, 19 aa, synthetic construct
FIRNRARGYTSDHNPSVKG

SEQ ID NO: 56 - protein, 8 aa, synthetic construct
RNRARGYT

SEQ ID NO: 57 - protein, 10 aa, synthetic construct
DRPSYYVLDY

SEQ ID NO: 58 - protein, 17 aa, synthetic construct
KSSQSLFNVRSRKNYLA

SEQ ID NO: 59 - protein, 7 aa, synthetic construct
WASTRES

SEQ ID NO: 60 - protein, 8 aa, synthetic construct
KQSYDLFT

SEQ ID NO: 61 - protein, 121 aa, synthetic construct
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSS

SEQ ID NO: 62 - protein, 112 aa, synthetic construct
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIK

SEQ ID NO: 63 - protein, 447 aa, synthetic construct
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCRVRCPRCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 64 - protein, 219 aa, synthetic construct
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC